(19) United States
(12) Patent Application Publication
(10) Pub. No.: US 2018/0360040 A1
(43) Pub. Date: Dec. 20, 2018

(54) PESTICIDAL COMPOUNDS AND THEIR USES

(71) Applicant: SYNGENTA PARTICIPATIONS AG, Basel (CH)

(72) Inventors: Ottmar Franz Hueter, Stein (CH); Andrew Edmunds, Stein (CH); Mark Hoppe, Stein (CH); Philip Wege, Bracknell (GB); Peter Maienfisch, Stein (CH); Michael Drysdale Turnbull, Bracknell (GB)

(73) Assignee: SYNGENTA PARTICIPATIONS AG, Basel (CH)

(21) Appl. No.: 15/780,095

(22) PCT Filed: Jan. 12, 2016
(86) PCT No.: PCT/EP2016/079442

§ 371 Date: May 30, 2018

(30) Foreign Application Priority Data
Dec. 3, 2015 (EP) 15197836.8

(51) Int. Cl. A01N 43/50; C07D 271/07; A01N 43/82; C07D 233/48; C07D 413/04; C07D 417/04

(52) U.S. Cl. CPC A01N 43/50; C07D 271/07; C07D 417/04; C07D 233/48; C07D 413/04; A01N 43/82

(57) ABSTRACT

The present invention concerns certain cyclic derivatives of guanidine compounds and their use in protecting plants, their use to control mosquitoes, and vector control management methods or control solutions comprising such a cyclic derivative of guanidine compounds, in particular the invention relates to a substrate, to a composition, comprising a cyclic derivative of guanidine compounds for controlling mosquitoes.

The present invention is in the technical field of pest control, in particular insect control and/or mosquito control, in particular insect vector control, with certain active cyclic derivatives of guanidine compounds and salts thereof. More specifically, the present invention relates to methods of controlling mosquitoes and to substrates, products, compositions and integrated mosquito management solutions for controlling mosquitoes, each comprising certain mosquitocidally active cyclic derivatives of guanidine compounds, and to certain active cyclic derivatives of guanidine compounds and derivatives thereof, to processes for preparing these compounds, to insecticidal compositions comprising these compounds and to methods of using these compounds to control insect pests.

There is a continuing need to find new biologically active compounds as well as new biologically active compounds displaying superior properties for use as agrochemical active ingredients for protecting plants, for example greater biological activity, improved crop yields, different spectrum of activity, increased safety profile, increased biodegradability and mitigate or control resistance development by pests.

Further, mosquito control manages the population of mosquitoes to reduce their damage to human health, economies, and enjoyment. Mosquito control is a vital public-health practice throughout the world and especially in the tropics because mosquitoes spread many diseases, such as malaria (Wikipedia contributors, “Mosquito control”, Wikipedia).

Therefore, there is an ongoing search for insecticides, in particular for control of mosquitoes, especially for mosquitoes having developed resistance against pyrethroids.

WO2003053917 and JP 2005008583 describe certain guanidine compounds for pest control and WO2005058311, WO2006065277, WO2008103351, WO2010021680, WO2010105179, WO2006009653 and WO2001044200 certain compounds, for example, as inhibitors of beta-secretase.

With the present invention, it has now been found that certain cyclic derivatives of guanidine compounds and salts thereof have useful pest control characteristics, such as insecticidal, in particular, mosquitocidal, activity, and are surprisingly useful for controlling mosquitoes and for decreasing mosquito vector populations.

Accordingly, the present invention, in one aspect, provides the use of one or more compounds of formula (I), for controlling mosquitoes

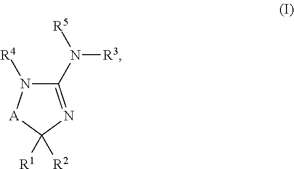

wherein

A represents O or CH₂;

R¹ is aryl; optionally substituted aryl; heteroaryl; optionally substituted heteroaryl; or cyclo-propyl;

R² is C₁-C₆-alkyl; optionally substituted C₁-C₆-alkyl; C₂-C₆-alkenyl; optionally substituted C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; optionally substituted C₃-C₅-cycloalkyl in which the cycloalkyl ring contains 1 oxygen heteroatom; aryl; optionally substituted aryl; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl;

R³ is C₁-C₈-alkyl; C₁-C₈-haloalkyl; C₂-C₈-alkenyl; C₂-C₈-alkynyl; C₂-C₈-haloalkenyl; C₃-C₁₀-cycloalkyl; C₃-C₁₀-halocycloalkyl; C₃-C₈-cycloalkyl in which the cycloalkyl ring contains 1 oxygen heteroatom; optionally substituted C₃-C₈-cycloalkyl in which the cycloalkyl ring contains 1 oxygen heteroatom; aryl; or haloaryl;

R⁴ is H; C₁-C₆-alkyl; halogen or CN substituted C₁-C₆-alkyl; C₂-C₆-alkenyl; or C₁-C₆-alkoxy;

R⁵ is H; C₁-C₆-alkyl; halogen or CN substituted C₁-C₆-alkyl; C₂-C₆-alkenyl; C₁-C₄-alkyl, C₁-C₄-haloalkyl, halogen or CN substituted C₂-C₆-alkenyl; C₂-C₆-alkynyl; or C₁-C₄-alkyl, C₁-C₄-haloalkyl, halogen or CN substituted C₂-C₆-alkynyl; or an agrochemically acceptable salt or N-oxide thereof.

In a second aspect, the present invention provides a compound of formula (I-A1) (group A),

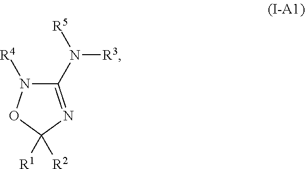

wherein R¹, R², R⁴ and R⁵ are as defined for formula (I) herein and R³ is selected from n-C₁-C₈-alkyl, C₃-C₈-fluoroalkyl, C₂-C₈-alkenyl, C₂-C₈-alkynyl, C₂-C₈-haloalkenyl, C₃-C₁₀-cycloalkyl, C₃-C₁₀-halocycloalkyl, C₃-C₁₀-cycloalkyl in which the cycloalkyl ring contains 1 oxygen heteroatom, phenyl, and halophenyl.

In a further aspect, the present invention also provides a group of compounds of formula (I-A1) (group B), wherein R¹, R², R⁴ and R⁵ are as defined for formula (I) above and R³ is either C(C₂H₅)₃, or C(CH₃)₂—CH₂—C(CH₃)₃.

In a further aspect, the present invention also provides a group of compounds of formula (I-A1) (group C), wherein R², R⁴ and R⁵ are as defined for formula (I) above, R¹ is substituted aryl; heteroaryl; optionally substituted heteroaryl; or cyclo-propyl and R³ is selected from CH(CH₃)₂, C(CH₃)₂(C₂H₅), C(CH₃)(C₂H₅)₂, 1-methyl-cyclopropyl and CH₂CF₃.

In a further aspect, the present invention also provides a group of compounds of formula (I-A1) (group D), wherein R¹, R³, R⁴ and R⁵ are as defined for formula (I) above and R² is selected from 3-methyl-oxetan-3-yl, CH₂CF₃ and n-pentyl.

In a further aspect, the present invention also provides a group of compounds of formula (I-A1) (group E), wherein R³, R⁴ and R⁵ are as defined for formula (I) above, R¹ is substituted aryl; heteroaryl; optionally substituted heteroaryl; or cyclo-propyl and R² is methyl, ethyl, n-propyl, n-hexyl, phenylethyl, and CF₃.

In a further aspect, the present invention also provides a group of compounds of formula (I-A1) (group F), wherein R³, R⁴ and R⁵ are as defined for formula (I) above, R¹ is substituted aryl, heteroaryl, optionally substituted heteroaryl and
(i) R² is n-butyl with the proviso that the substituent on the aryl of R¹ is not 4-chloro or not 4-OCF₃; or
(ii) R² is cyclo-pentyl with the proviso that the substituent on the aryl of R¹ is not 4-chloro.

In a further aspect, the present invention also provides a group of compounds of formula (I-A1) (group G), wherein R³, R⁴ and R⁵ are as defined for formula (I) above, R¹ is substituted aryl, heteroaryl, or optionally substituted heteroaryl and R² is C₃-C₅-cycloalkyl with the proviso that the substituent on the aryl of R¹ is 2-fluoro.

In a further aspect, the present invention also provides a group of compounds of formula (I-A1) (group H), wherein R¹, R³, R⁴ and R⁵ are as defined for formula (I) above, and R² is cyclo-propylmethyl with the proviso that the substituent on the aryl of R¹ is not 4-chloro.

The compounds of the invention (for example, of formulae (I), (I-1), (I-2), (I-A1) may exist in different geometric or optical isomers or tautomeric forms. This invention covers all such isomers and tautomers and mixtures thereof in all proportions as well as isotopic forms such as deuterated compounds.

The compounds of the invention and, where appropriate, the tautomers thereof, in each case in free form or in salt form, can be present in the form of one of the isomers which are possible or as a mixture of these, for example in the form of pure isomers, such as antipodes, or as isomer mixtures, such as enantiomer mixtures, for example racemates, or racemate mixtures, depending on the absolute configuration of asymmetric carbon atom which occurs in the molecule; the invention relates to the pure isomers and also to all isomer mixtures which are possible and is to be understood in each case in this sense hereinabove and hereinbelow, even when stereochemical details are not mentioned specifically in each case. This invention accordingly covers all such isomers and tautomers and mixtures thereof in all proportions as well as isotopic forms such as deuterated compounds. As an example, the compounds of the invention may contain one or more asymmetric carbon atoms, for example, if R¹ is different from R², the compounds of formula (I) may exist as enantiomers or as mixtures of such.

The invention also covers salts and N-oxides of each compound for formula (I).

One skilled in the art also recognizes that because in the environment and under physiological conditions salts of chemical compounds are in equilibrium with their corresponding non salt forms, salts share the biological utility of the non salt forms.

Thus a wide variety of salts of compounds of the invention (and active ingredients used in combination with the active ingredients of the invention) may be useful for control of invertebrate pests and animal parasites. Salts amongst agriculturally and/or physiologically tolerable salts include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

Suitable amongst agriculturally and/or physiologically tolerable salts can also be the salts of those cations which do not adversely affect the pesticidal and/or parasiticidal action of the compounds of formula (I). Thus, especially suitable cations are the ions of the alkali metals including sodium, potassium and lithium, of the alkaline earth metals including calcium and magnesium, and of the transition metals including manganese, copper, iron, zinc, cobalt, lead, silver, nickel, and also ammonium or organic ammonium including monoalkylammonium, dialkylammonium, trialkylammonium, tetraalkylammonium, monoalkenylammonium, dialkenylammonium, trialkenylammonium, monoalkynylammonium, dialkynyla monium, monoalkanolammonium, dialkanolammonium, C₅-C₆-cycloalkylammonium, piperidinium, morpholinium, pyrrolidinium, or benzylammonium, moreover phosphonium ions, sulfonium ions, preferably tri(C₁-C₄-alkyl) sulfonium and sulfoxonium ions, preferably tri (C₁-C₄-alkyl) sulfoxonium.

Alkyl groups (either alone or as part of a larger group, such as alkoxy-) can be in the form of a straight or branched chain and are, for example, methyl, ethyl, propyl, prop-2-yl, butyl, but-2-yl, or 2-methyl-prop-2-yl. The alkyl group (either alone or as part of a larger group, such as alkoxy-), in each embodiment of the invention, is preferably C₁-C₃-alkyl, more preferably C₁-C₂-alkyl, especially methyl group. In the instance of alkoxy, examples are methoxy, ethoxy, propoxy, n-butoxy, isobutoxy and also their isomeric groups; preferably, independent of other embodiments, methoxy and ethoxy, especially methoxy.

Halogen is fluorine, chlorine, bromine or iodine; halogen, in each embodiment of the invention, is fluorine, chlorine, or bromine; especially fluorine or chlorine.

Haloalkyl groups (either alone or as part of a larger group, such as haloalkoxy-) are alkyl groups which are substituted by one or more of the same or different halogen atoms and are, for example, fluoromethyl, difluoromethyl, trifluoromethyl, chlorodifluoromethyl and 2,2,2-trifluoro-ethyl. The haloalkyl group (either alone or as part of a larger group, such as haloalkoxy-), in each embodiment of the invention, is preferably trifluromethyl. In instance of haloalkoxy, examples are fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2,2,2-trifluoro-ethoxy, 2-chloroethoxy and trifluoromethoxy.

Cycloalkyl groups can be mono- or bi-cyclic and are, for example, cyclopropyl, cyclobutyl, cyclopentyl, cyclohexyl, adamantyl. In an embodiment, the cycloalkyl group is mono-cyclic. The C₃-C₁₀-cycloalkyl group, in each embodiment of the invention, is preferably a C₃-C₅-cycloakyl, more preferably a C₃-C₄-cycloalkyl group, especially a C₃-cycloalkyl group. Where a cycloalkyl moiety is said to be substituted, the cycloalkyl moiety is preferably substituted by one to four substituents, most preferably by one to three substituents, and the substituent can be on the carbon atom of the cycloalkyl group connected to remainder of the compound or another carbon atom, preferably the substituent is on the carbon atom of the cycloalkyl group connected to the remainder of the compound. Where a cycloalkyl moiety is said to contain 1 oxygen heteroatom, the heteroatom is not itself connecting to the remainder of the compound, an example of the invention being 3-methyl-oxetan-3-yl or substituent R³ in compound 1.152 in Table P1 below.

Cycloalkylalkyl groups are mono cyclic attached to an alkyl group, and the number of carbon atoms refers to the carbon atoms in the cyclic group and also the alkyl group—therefore a C₄-cycloaklyl would be cyclopropylmethyl. In the event the cycloalkylalkyl group is substituted, the substituent can be on the cycloalkyl group or alkyl group.

Aryl groups (either alone or as part of a larger group, such as aryl-alkylene) are aromatic ring systems which can be in mono-, bi- or tricyclic form. Examples of such rings include phenyl, naphthyl, anthracenyl, indenyl or phenanthrenyl. Preferred aryl groups are phenyl and naphthyl, phenyl being most preferred. Where an aryl moiety is said to be substituted, the aryl moiety is preferably substituted by one to four substituents, most preferably by one to three substituents.

Heteroaryl groups (either alone or as part of a larger group, such as heteroaryl-alkylene) are aromatic ring systems containing at least one heteroatom and consisting either of a single ring or of two or more fused rings. Preferably, single rings will contain up to three heteroatoms and bicyclic systems up to four heteroatoms, which in each instance will preferably be chosen from nitrogen, oxygen and sulfur. Examples of monocyclic groups include pyridyl, pyridazinyl, pyrimidinyl, pyrazinyl, pyrrolyl, pyrazolyl, imidazolyl, triazolyl (e.g., 1.2.4 triazoyl), furanyl, thiophenyl, oxazolyl, isoxazolyl, oxadiazolyl, thiazolyl, isothiazolyl and thiadiazolyl. Examples of bicyclic groups include purinyl, quinolinyl, cinnolinyl, quinoxalinyl, indolyl, indazolyl, benzimidazolyl, benzothiophenyl and benzothiazolyl. Monocyclic heteroaryl groups are preferred, pyridyl and thiazolyl being most preferred. Where a heteroaryl moiety is said to be substituted, the heteroaryl moiety is preferably substituted by one to four substituents, most preferably by one to three substituents.

A preferred group of compounds of the first aspect of the invention are represented by the compounds of formula (I-1)

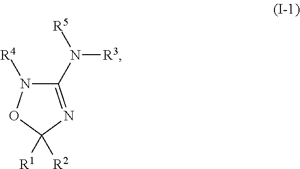

wherein R¹, R², R³, R⁴ and R⁵ are as defined herein.

A further preferred group of compounds of the first aspect of the invention are represented by the compounds of formula (I-2)

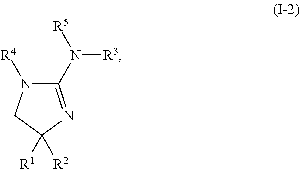

wherein R¹, R², R³, R⁴ and R⁵ are as defined herein.

R¹, in respect of formulae (I), (I-1), (I-2) and (I-A1) (groups A, B, D & H) and independent of the formulae in which it appears herein and independent of the other substituents, is preferably aryl; aryl substituted by 1 to 3 substituents independently selected from C₁-C₄-alkyl, C₁-C₄-alkoxy, C₁-C₄-haloalkoxy, C₁-C₄-haloalkyl, halogen and CN; heteroaryl containing 1 to 3 heteroatoms independently selected from nitrogen, oxygen and sulfur; heteroaryl thereof substituted by 1 to 3 substituents independently selected from C₁-C₄-alkyl, C₁-C₄-alkoxy, C₁-C₄-haloalkyl, halogen and CN; or cyclo-propyl; more preferably aryl or aryl substituted by 1 to 3 substituents independently selected from C₁-C₄-alkyl, C₁-C₄-alkoxy, C₁-C₄-haloalkoxy, C₁-C₄-haloalkyl, halogen and CN; most preferably phenyl or phenyl substituted by 1 to 3 substituents independently selected from C₁-C₄-alkyl, C₁-C₄-alkoxy, C₁-C₄-haloalkoxy, C₁-C₄-haloalkyl, halogen and CN.

R¹, in respect of formulae (I-A1) (groups C, E, F & G) and independent of the formulae in which it appears herein and independent of the other substituents, is preferably aryl substituted by 1 to 3 substituents independently selected from C₁-C₄-alkyl, C₁-C₄-alkoxy, C₁-C₄-haloalkoxy, C₁-C₄-haloalkyl, halogen and CN; heteroaryl containing 1 to 3 heteroatoms independently selected from nitrogen, oxygen and sulfur; the heteroaryl thereof substituted by 1 to 3 substituents independently selected from C₁-C₄-alkyl, C₁-C₄-alkoxy, C₁-C₄-haloalkyl, halogen and CN; or cyclo-propyl; more preferably aryl substituted by 1 to 3 substituents independently selected from C₁-C₄-alkyl, C₁-C₄-alkoxy, C₁-C₄-haloalkoxy, C₁-C₄-haloalkyl, halogen and CN; most preferably phenyl substituted by 1 to 3 substituents independently selected from C₁-C₄-alkyl, C₁-C₄-alkoxy, C₁-C₄-haloalkoxy, C₁-C₄-haloalkyl, halogen and CN.

R², in respect of formulae (I), (I-1), (I-2) and (I-A1) (groups A to C) and independent of the other substituents, is preferably C₁-C₆-alkyl; halogen or CN substituted C₁-C₆-alkyl; C₂-C₆-alkenyl; C₁-C₄-haloalkyl, halogen or CN substituted C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; C₁-C₄-alkyl, C₁-C₄-haloalkyl, halogen or CN substituted C₃-C₈-cycloalkyl in which the cycloalkyl ring contains 1 oxygen heteroatom; aryl; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; more preferably C₁-C₆-alkyl; C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; C₁-C₄-alkyl, C₁-C₄-haloalkyl, halogen or CN substituted C₃-C₈-cycloalkyl in which the cycloalkyl ring contains 1 oxygen heteroatom; aryl; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; most preferably C₁-C₆-alkyl; C₃-C₈-cycloalkyl; aryl; aryl substituted by 1 to 3 substituents independently selected from C₁-C₄-alkyl, C₁-C₄-alkoxy, C₁-C₄-haloalkoxy C₁-C₄-haloalkyl, halogen and CN; arylalkyl; or C₄-C₈-cycloalkylalkyl.

R², in respect of formula (I-A1) (group D), and independent of the other substituents, is preferably n-pentyl.

R², in respect of formula (I-A1) (group E), and independent of the other substituents, is preferably n-propyl.

R², in respect of formula (I-A1) (group F), and independent of the other substituents, is preferably n-butyl with the proviso that the substituent on the aryl of R¹ is not 4-chloro or not 4-OCF₃.

R², in respect of formula (I-A1) (group G), and independent of the other substituents, is preferably C₃-C₆-cycloalkyl.

R³, in respect of formulae (I), (I-1) and (I-2) and independent of the other substituents, is preferably a branched C₁-C₈-alkyl; C₁-C₈-haloalkyl; C₂-C₈-alkenyl; C₂-C₈-alkynyl; C₃-C₁₀-cycloalkyl; C₃-C₁₀-halocycloalkyl; C₁-C₄-alkyl, C₁-C₄-haloalkyl, halogen or CN substituted C₃-C₈-cycloalkyl in which the cycloalkyl ring contains 1 oxygen heteroatom; aryl; or haloaryl; more preferably, a branched C₁-C₈-alkyl, C₁-C₈-haloalkyl, or C₃-C₁₀-halocycloalkyl; most preferably, a branched C₁-C₆-alkyl, C₁-C₆-haloalkyl, or C₃-C₈-halocycloalkyl.

R³, in respect of formulae (I-A1) and independent of the other substituents, is preferably n-C₁-C₈-alkyl, C₃-C₈-fluoroalkyl, C₃-C₁₀-cycloalkyl, C₃-C₁₀-halocycloalkyl, C₁-C₄-alkyl, C₁-C₄-haloalkyl, halogen or CN substituted C₃-C₁₀-cycloalkyl in which the cycloalkyl ring contains 1 oxygen heteroatom, phenyl or halophenyl; more preferably, n-C₂-C₄-alkyl, branched C₃-C₈-fluoroalkyl, C₃-C₈-cycloalkyl, C₃-C₈-halocycloalkyl, C₃-C₈-cycloalkyl in which the cycloalkyl ring contains 1 oxygen heteroatom, phenyl or chlorophenyl; most preferably C₃-C₈-fluoroalkyl, or C₃-C₈-halocycloalkyl.

R⁴, independent of the formulae (I), (I-1) and (I-A1) and independent of the other substituents, is preferably C₁-C₆-alkyl; halogen or CN substituted C₁-C₆-alkyl; or C₂-C₆-alkenyl; more preferably, C₁-C₆-alkyl or C₁-C₆-haloalkyl; most preferably C₁-C₆-alkyl.

R⁴, in respect of formula (I-2) and independent of the other substituents, is preferably C₁-C₆-alkyl; halogen or CN substituted C₁-C₆-alkyl; C₂-C₆-alkenyl; or C₁-C₆-alkoxy; more preferably, C₁-C₆-alkyl, C₁-C₆-haloalkyl or C₁-C₆-alkoxy; most preferably C₁-C₆-alkyl.

R⁵, independent of the formulae in which it appears herein and independent of the other substituents, is preferably H; C₁-C₆-alkyl; halogen or CN substituted C₁-C₆-alkyl; C₂-C₆-alkenyl; C₁-C₄-alkyl, C₁-C₄-haloalkyl, halogen or CN substituted C₂-C₆-alkenyl; C₂-C₆-alkynyl; C₁-C₄-alkyl, C₁-C₄-haloalkyl, halogen or CN substituted C₂-C₆-alkynyl; aryl; or C₁-C₄-alkyl, C₁-C₄-haloalkyl, halogen or CN substituted aryl; more preferably, H or C₁-C₆-alkyl; most preferably H.

In an embodiment of the first aspect of the invention, the compound of formula (I) is represented by the compounds of formula (I-1), wherein

R¹ is aryl; aryl substituted by 1 to 3 substituents independently selected from C₁-C₄-alkyl, C₁-C₄-alkoxy, C₁-C₄-haloalkoxy, C₁-C₄-haloalkyl, halogen and CN; heteroaryl containing 1 to 3 heteroatoms independently selected from nitrogen, oxygen and sulfur; heteroaryl thereof substituted by 1 to 3 substituents independently selected from C₁-C₄-alkyl, C₁-C₄-alkoxy, C₁-C₄-haloalkyl, halogen and CN; or cyclo-propyl; more preferably aryl or aryl substituted by 1 to 3 substituents independently selected from C₁-C₄-alkyl, C₁-C₄-alkoxy, C₁-C₄-haloalkoxy, C₁-C₄-haloalkyl, halogen and CN; most preferably phenyl or phenyl substituted by 1 to 3 substituents independently selected from C₁-C₄-alkyl, C₁-C₄-alkoxy, C₁-C₄-haloalkoxy, C₁-C₄-haloalkyl, halogen and CN; especially phenyl, phenyl substituted by 1 to 3 substituents independently selected from halogen, cyano, C₁-C₄-alkyl, C₁-C₄-haloalkyl, C₁-C₄-haloalkoxy, and C₁-C₄-alkoxy;

R² is C₁-C₆-alkyl; halogen or CN substituted C₁-C₆-alkyl; C₂-C₆-alkenyl; C₁-C₄-haloalkyl, halogen or CN substituted C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; C₁-C₄-alkyl, C₁-C₄-haloalkyl, halogen or CN substituted C₃-C₈-cycloalkyl in which the cycloalkyl ring contains 1 oxygen heteroatom; aryl; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; more preferably C₁-C₆-alkyl; C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; C₁-C₄-alkyl, C₁-C₄-haloalkyl, halogen or CN substituted C₃-C₈-cycloalkyl in which the cycloalkyl ring contains 1 oxygen heteroatom; aryl; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; most preferably C₁-C₆-alkyl; C₃-C₈-cycloalkyl; aryl; aryl substituted by 1 to 3 substituents independently selected from C₁-C₄-alkyl, C₁-C₄-alkoxy, C₁-C₄-haloalkoxy C₁-C₄-haloalkyl, halogen and CN; arylalkyl; or C₄-C₈-cycloalkylalkyl; especially C₁-C₄-alkyl, cyclopropyl, phenyl, benzyl, phenylethyl, cyclopropylmethyl, or mono-substituted halophenyl;

R³ is a branched C₁-C₈-alkyl; C₁-C₈-haloalkyl; C₂-C₈-alkenyl; C₂-C₈-alkynyl; C₃-C₁₀-cycloalkyl; C₃-C₁₀-halocycloalkyl; C₁-C₄-alkyl substituted C₃-C₈-cycloalkyl in which the cycloalkyl ring contains 1 oxygen heteroatom; aryl; or haloaryl; more preferably, a branched C₁-C₈-alkyl, C₁-C₈-haloalkyl, or C₃-C₁₀-halocycloalkyl; most preferably, a branched C₁-C₆-alkyl, C₁-C₆-haloalkyl, or C₃-C₈-halocycloalkyl; especially t-Butyl, C(CH₃)₂(C₂H₅), or C(CH₃)₂(CF₃);

R⁴ is C₁-C₆-alkyl; halogen or CN substituted C₁-C₆-alkyl; C₂-C₆-alkenyl; or C₁-C₆-alkoxy; more preferably, C₁-C₆-alkyl or C₁-C₆-alkoxy; most preferably C₁-C₆-alkyl; especially methyl or ethyl; and

R⁵ is H; C₁-C₆-alkyl; halogen or CN substituted C₁-C₆-alkyl; C₂-C₆-alkenyl; C₁-C₄-alkyl, C₁-C₄-haloalkyl, halogen or CN substituted C₂-C₆-alkenyl; C₂-C₆-alkynyl; C₁-C₄-alkyl, C₁-C₄-haloalkyl, halogen or CN substituted C₂-C₆-alkynyl; aryl; or C₁-C₄-alkyl, C₁-C₄-haloalkyl, halogen or CN substituted aryl; more preferably, H or C₁-C₆-alkyl; most preferably H.

In a preferred embodiment of the first aspect of the invention, the compound of formula (I) is represented by the compounds of formula (I-1), wherein

R¹ is phenyl, phenyl substituted by 1 to 3 substituents independently selected from halogen, cyano, C₁-C₄-alkyl, C₁-C₄-haloalkyl, C₁-C₄-haloalkoxy, and C₁-C₄-alkoxy;
R² is C₁-C₄-alkyl, cyclopropyl, phenyl, benzyl, phenylethyl, cyclopropylmethyl, or mono-substituted halophenyl;
R³ is t-Butyl, C(CH₃)₂(C₂H₅), or C(CH₃)₂(CF₃);
R⁴ is methyl or ethyl; and

### R⁵ is H.

In a preferred embodiment of the first aspect of the invention, the compound of formula (I) is represented by the compounds of formula (I-2), wherein

R¹ is aryl, or aryl substituted by 1 to 3 substituents independently selected from halogen, cyano, C₁-C₄-alkyl, C₁-C₄-haloalkyl, C₁-C₄-haloalkoxy, and C₁-C₄-alkoxy;
R² is C₁-C₄-alkyl, cyclopropyl, aryl or aryl substituted by 1 to 3 substituents independently selected from halogen, cyano, C₁-C₄-alkyl, C₁-C₄-haloalkyl, C₁-C₄-haloalkoxy, and C₁-C₄-alkoxy;
R³ is C₁-C₅-alkyl or C₁-C₄-haloalkyl;
R⁴ is C₁-C₄-alkyl, C₁-C₄-haloalkyl or C₁-C₄-alkoxy; and
R⁵ is H or C₁-C₄-alkyl.

A preferred group of compounds of formula (I-A1) of the first aspect of the invention is where

R¹ is aryl; aryl substituted by 1 to 3 substituents independently selected from C₁-C₄-alkyl, C₁-C₄-alkoxy, C₁-C₄-haloalkoxy, C₁-C₄-haloalkyl, halogen and CN; heteroaryl containing 1 to 3 heteroatoms independently selected from nitrogen, oxygen and sulfur; heteroaryl thereof substituted by 1 to 3 substituents independently selected from C₁-C₄-alkyl, C₁-C₄-alkoxy, C₁-C₄-haloalkyl, halogen and CN; or cyclo-propyl; more preferably aryl or aryl substituted by 1 to 3 substituents independently selected from C₁-C₄-alkyl, C₁-C₄-alkoxy, C₁-C₄-haloalkoxy, C₁-C₄-haloalkyl, halogen and CN; most preferably phenyl or phenyl substituted by 1 to 3 substituents independently selected from C₁-C₄-alkyl, C₁-C₄-alkoxy, C₁-C₄-haloalkoxy, C₁-C₄-haloalkyl, halogen and CN;

R² is C₁-C₆-alkyl; halogen or CN substituted C₁-C₆-alkyl; C₂-C₆-alkenyl; C₁-C₄-haloalkyl, halogen or CN substituted C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; C₁-C₄-alkyl, C₁-C₄-haloalkyl, halogen or CN substituted C₃-C₈-cycloalkyl in which the cycloalkyl ring contains 1 oxygen heteroatom; aryl; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; more preferably C₁-C₆-alkyl; C₂-C₆-alkenyl; C₃-C₆-cycloalkyl; C₁-C₄-alkyl, C₁-C₄-haloalkyl, halogen or CN substituted C₃-C₆-cycloalkyl; C₃-C₆-cycloalkyl in which the cycloalkyl ring contains 1 oxygen heteroatom; C₁-C₄-alkyl, C₁-C₄-haloalkyl, halogen or CN substituted C₃-C₆-cycloalkyl in which the cycloalkyl ring contains 1 oxygen heteroatom; aryl; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; most preferably C₁-C₆-alkyl; C₃-C₆-cycloalkyl; aryl; aryl substituted by 1 to 3 substituents independently selected from C₁-C₄-alkyl, C₁-C₄-alkoxy, C₁-C₄-haloalkoxy C₁-C₄-haloalkyl, halogen and CN; aryl-C₁-C₄-alkyl; or C₄-C₈-cycloalkylalkyl;

R³ is n-C₁-C₈-alkyl, C₃-C₈-fluoroalkyl, C₃-C₁₀-cycloalkyl, C₃-C₁₀-halocycloalkyl, C₃-C₁₀-cycloalkyl in which the cycloalkyl ring contains 1 oxygen heteroatom, phenyl or halophenyl; more preferably, n-C₂-C₄-alkyl, branched C₃-C₈-fluoroalkyl, C₃-C₆-cycloalkyl, C₃-C₆-halocycloalkyl, C₃-C₆-cycloalkyl in which the cycloalkyl ring contains 1 oxygen heteroatom, phenyl or chlorophenyl; most preferably C₃-C₆-fluoroalkyl, or C₃-C₆-halocycloalkyl;

R⁴ is C₁-C₆-alkyl; halogen or CN substituted C₁-C₆-alkyl; C₂-C₆-alkenyl; or C₁-C₆-alkoxy; more preferably, C₁-C₆-alkyl or C₁-C₆-haloalkyl; most preferably C₁-C₆-alkyl; and

R⁵ is preferably H; C₁-C₆-alkyl; halogen or CN substituted C₁-C₆-alkyl; C₂-C₆-alkenyl; C₁-C₄-alkyl, C₁-C₄-haloalkyl, halogen or CN substituted C₂-C₆-alkenyl; C₂-C₆-alkynyl; C₁-C₄-alkyl, C₁-C₄-haloalkyl, halogen or CN substituted C₂-C₆-alkynyl; aryl; or C₁-C₄-alkyl, C₁-C₄-haloalkyl, halogen or CN substituted aryl; more preferably, H or C₁-C₆-alkyl; most preferably H.

A further preferred group of compounds of formula (I-A1) of the first aspect of the invention is where

R¹ is 2-fluoro substituted aryl, heteroaryl containing 1 to 3 heteroatoms independently selected from nitrogen, oxygen and sulfur; heteroaryl thereof substituted by 1 to 3 substituents independently selected from C₁-C₄-alkyl, C₁-C₄-alkoxy, C₁-C₄-haloalkyl, halogen and CN; or cyclo-propyl; more preferably 2-fluoro substituted aryl; most preferably 2-fluoro substituted phenyl;
R² is C₃-C₈-cycloalkyl; preferably C₃-C₆-cycloalkyl; more preferably cyclopropyl;
R³ is a branched C₁-C₈-alkyl; C₁-C₈-haloalkyl; C₂-C₈-alkenyl; C₂-C₈-alkynyl; C₃-C₁₀-cycloalkyl; C₃-C₁₀-halocycloalkyl; C₃-C₈-cycloalkyl in which the cycloalkyl ring contains 1 oxygen heteroatom; aryl; or haloaryl; more preferably, a branched C₁-C₈-alkyl, C₁-C₈-haloalkyl, or C₃-C₁₀-halocycloalkyl; most preferably, a branched C₁-C₆-alkyl, C₁-C₆-haloalkyl, or C₃-C₈-halocycloalkyl; especially t-butyl, C(CH₃)₂(C₂H₅), or C(CH₃)₂(CF₃);
R⁴ is C₁-C₆-alkyl; halogen or CN substituted C₁-C₆-alkyl; C₂-C₆-alkenyl; or C₁-C₆-alkoxy; more preferably, C₁-C₆-alkyl or C₁-C₆-haloalkyl; most preferably C₁-C₆-alkyl; and
R⁵ is preferably H; C₁-C₆-alkyl; C₁-C₄-haloalkyl, halogen or CN substituted C₁-C₆-alkyl; C₂-C₆-alkenyl; C₁-C₄-alkyl, C₁-C₄-haloalkyl, halogen or CN substituted C₂-C₆-alkenyl; C₂-C₆-alkynyl; C₁-C₄-alkyl, C₁-C₄-haloalkyl, halogen or CN substituted C₂-C₆-alkynyl; aryl; or C₁-C₄-alkyl, C₁-C₄-haloalkyl, halogen or CN substituted aryl; more preferably, H or C₁-C₆-alkyl; most preferably H.

Another preferred group of compounds of formula (I-A1) of the first aspect of the invention is where

R¹ is aryl; aryl substituted by 1 to 3 substituents independently selected from C₁-C₄-alkyl, C₁-C₄-alkoxy, C₁-C₄-haloalkoxy, C₁-C₄-haloalkyl, halogen and CN; heteroaryl containing 1 to 3 heteroatoms independently selected from nitrogen, oxygen and sulfur; heteroaryl thereof substituted by 1 to 3 substituents independently selected from C₁-C₄-alkyl, C₁-C₄-alkoxy, C₁-C₄-haloalkyl, halogen and CN; or cyclo-propyl; more preferably aryl or aryl substituted by 1 to 3 substituents independently selected from C₁-C₄-alkyl, C₁-C₄-alkoxy, C₁-C₄-haloalkoxy, C₁-C₄-haloalkyl, halogen and CN; most preferably phenyl or phenyl substituted by 1 to 3 substituents independently selected from C₁-C₄-alkyl, C₁-C₄-alkoxy, C₁-C₄-haloalkoxy, C₁-C₄-haloalkyl, halogen and CN;
R² is C₁-C₆-alkyl; halogen or CN substituted C₁-C₆-alkyl; C₂-C₆-alkenyl; C₁-C₄-haloalkyl, halogen or CN substituted C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; C₁-C₄-alkyl, C₁-C₄-haloalkyl, halogen or CN substituted C₃-C₈-cycloalkyl in which the cycloalkyl ring contains 1 oxygen heteroatom; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; more preferably C₁-C₆-alkyl; C₂-C₆-alkenyl; C₃-C₈-cycloalkyl; C₁-C₄-alkyl, C₁-C₄-haloalkyl, halogen or CN substituted C₃-C₈-cycloalkyl; C₃-C₈-cycloalkyl in which the cycloalkyl ring contains 1 oxygen heteroatom; C₁-C₄-alkyl, C₁-C₄-haloalkyl, halogen or CN substituted C₃-C₈-cycloalkyl in which the cycloalkyl ring contains 1 oxygen heteroatom; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; most preferably C₁-C₆-alkyl; C₃-C₈-cycloalkyl; aryl substituted by 1 to 3 substituents independently selected from C₁-C₄-alkyl, C₁-C₄-alkoxy, C₁-C₄-haloalkoxy C₁-C₄-haloalkyl, halogen and CN; arylalkyl; or C₄-C₈-cycloalkylalkyl;
R³ is CH(CH₃)₂, C(CH₃)₂(C₂H₅), C(CH₃)(C₂H₅)₂, 1-methyl-cyclopropyl or CH₂CF₃; preferably C(CH₃)₂(C₂H₅);
R⁴ is C₁-C₆-alkyl; halogen or CN substituted C₁-C₆-alkyl; C₂-C₆-alkenyl; or C₁-C₆-alkoxy; more preferably, C₁-C₆-alkyl or C₁-C₆-haloalkyl; most preferably C₁-C₆-alkyl; and
R⁵ is preferably H; C₁-C₆-alkyl; halogen or CN substituted C₁-C₆-alkyl; C₂-C₆-alkenyl; C₁-C₄-alkyl, C₁-C₄-haloalkyl, halogen or CN substituted C₂-C₆-alkenyl; C₂-C₆-alkynyl; C₁-C₄-alkyl, C₁-C₄-haloalkyl, halogen or CN substituted C₂-C₆-alkynyl; aryl; or C₁-C₄-alkyl, C₁-C₄-haloalkyl, halogen or CN substituted aryl; more preferably, H or C₁-C₆-alkyl; most preferably H.

In an embodiment of the first aspect, especially preferred compounds are

R¹ is phenyl or phenyl substituted by one to three substituents independently selected halogen atoms;
R² is C₂-C₅-alkyl or cyclopropyl
R³ is bridged C₃-C₅-alkyl, or C₃-C₅-haloalkyl
R⁴ is methyl; and

### R⁵ is H.

In an embodiment of the second aspect of the invention, the compound of formula (I) is represented by the compounds of formula (I-A1), wherein

R¹ is 2-fluoro phenyl;
R² is cyclopropyl,
R³ is t-butyl, C(CH₃)₂(C₂H₅) or C(CH₃)₂(CF₃);
R⁴ is methyl; and

### R⁵ is H.

In a further embodiment of the second aspect of the invention, the compound of formula (I) is represented by the compounds of formula (I-A1), wherein

R¹ is 4-fluoro phenyl;
R² is cyclopropyl;
R³ is C(CH₃)₂(C₂H₅), or C(CH₃)₂(CF₃);
R⁴ is methyl; and

### R⁵ is H.

In a further embodiment of the second aspect of the invention, the compound of formula (I) is represented by the compounds of formula (I-A1), wherein

R¹ is aryl substituted by 1 to 3 substituents independently selected from C₁-C₄-alkyl, C₁-C₄-alkoxy, C₁-C₄-haloalkoxy, C₁-C₄-haloalkyl, halogen and CN;
R² is n-propyl or n-butyl;
R³ is C₁-C₆-alkyl, or C₁-C₆-haloalkyl;
R⁴ is C₁-C₄-alkyl; and

### R⁵ is H.

Preferably, R¹ is phenyl substituted by 1 to 3 substituents independently selected from C₁-C₄-alkyl, C₁-C₄-alkoxy, C₁-C₄-haloalkoxy, C₁-C₄-haloalkyl, halogen and CN; R² is n-propyl or n-butyl or n-pentyl; R³ is t-butyl, C(CH₃)₂(C₂H₅), or C(CH₃)₂(CF₃); R⁴ is methyl; and R⁵ is H.

In a preferred embodiment, a group of compounds of group F are those wherein R³, R⁴ and R⁵ are as defined for formula (I) above, R¹ is substituted aryl, heteroaryl, optionally substituted heteroaryl, and R² is selected from n-butyl, cyclo-pentyl and cyclo-propylmethyl, with the proviso that the substituent on the aryl of R¹ is not 4-chloro or not 4-OCF₃.

In a further preferred embodiment, a group of compounds of group F are those wherein R³, R⁴ and R⁵ are as defined for formula (I) above, R¹ is substituted aryl, heteroaryl, optionally substituted heteroaryl, and R² is selected from n-butyl, cyclo-pentyl and cyclo-propylmethyl, with the proviso that the substituent on the aryl of R¹ is not 4-halogen or not 4-haloalkyl.

Specific examples of compounds of the present invention are represented by the formula (I-1) in the following Tables 1 to 14:

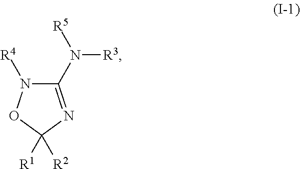

wherein R¹, R³, R⁴ and R⁵ are as defined below in Table X.

Each of Tables 1 to 14, which follow the Table X below, comprises 84 compounds of the formula (I-1) in which R¹, R³, R⁴ and R⁵ have the values given in each row in Table X, and R² has the value given in the relevant Tables 1 to 14. Thus compound 1. 1 corresponds to a compound of formula (I-1) where R¹, R³, R⁴ and R⁵ are as defined in row 1 of Table X and where R² is as defined in Table 1; compound 14.14 corresponds to a compound of formula (I-1) where R¹, R³, R⁴ and R⁵ are as defined in row 14 of Table X and where R² is as defined in Table 14; and so on.

TABLE X

| Comp. No | R¹ | R³ | R⁴ | R⁵ |
|---|---|---|---|---|
| X.1. | Ph | t-Bu | Me | H |
| X.2. | 2-F—Ph | t-Bu | Me | H |
| X.3. | 3-F—Ph | t-Bu | Me | H |
| X.4. | 4-F—Ph | t-Bu | Me | H |
| X.5. | 2-Cl—Ph | t-Bu | Me | H |
| X.6. | 3-Cl—Ph | t-Bu | Me | H |
| X.7. | 4-Cl—Ph | t-Bu | Me | H |
| X.8. | 2-Me—Ph | t-Bu | Me | H |
| X.9. | 3-Me—Ph | t-Bu | Me | H |
| X.10. | 4-Me—Ph | t-Bu | Me | H |
| X.11. | 2-OMe—Ph | t-Bu | Me | H |
| X.12. | 3-OMe—Ph | t-Bu | Me | H |
| X.13. | 4-OMe—Ph | t-Bu | Me | H |
| X.14. | 2-CF₃—Ph | t-Bu | Me | H |
| X.15. | 3-CF₃—Ph | t-Bu | Me | H |
| X.16. | 4-CF₃—Ph | t-Bu | Me | H |
| X.17. | 2-OCF₃—Ph | t-Bu | Me | H |
| X.18. | 3-OCF₃—Ph | t-Bu | Me | H |
| X.19. | 4-OCF₃—Ph | t-Bu | Me | H |
| X.20. | 2-CN—Ph | t-Bu | Me | H |
| X.21. | 3-CN—Ph | t-Bu | Me | H |
| X.22. | 4-CN—Ph | t-Bu | Me | H |
| X.23. | 3,4-F₂—Ph | t-Bu | Me | H |
| X.24. | 3,4-F₂—Ph | t-Bu | Me | H |
| X.25. | 3,4-F₂—Ph | t-Bu | Me | H |
| X.26. | 3,4-Cl₂—Ph | t-Bu | Me | H |
| X.27. | 3,4-Cl₂—Ph | t-Bu | Me | H |
| X.28. | 3,4-Cl₂—Ph | t-Bu | Me | H |
| X.29. | Ph | CMe₂Et | Me | H |
| X.30. | 2-F—Ph | CMe₂Et | Me | H |
| X.31. | 3-F—Ph | CMe₂Et | Me | H |
| X.32. | 4-F—Ph | CMe₂Et | Me | H |
| X.33. | 2-Cl—Ph | CMe₂Et | Me | H |
| X.34. | 3-Cl—Ph | CMe₂Et | Me | H |
| X.35. | 4-Cl—Ph | CMe₂Et | Me | H |
| X.36. | 2-Me—Ph | CMe₂Et | Me | H |
| X.37. | 3-Me—Ph | CMe₂Et | Me | H |
| X.38. | 4-Me—Ph | CMe₂Et | Me | H |
| X.39. | 2-OMe—Ph | CMe₂Et | Me | H |
| X.40. | 3-OMe—Ph | CMe₂Et | Me | H |
| X.41. | 4-OMe—Ph | CMe₂Et | Me | H |
| X.42. | 2-CF₃—Ph | CMe₂Et | Me | H |
| X.43. | 3-CF₃—Ph | CMe₂Et | Me | H |
| X.44. | 4-CF₃—Ph | CMe₂Et | Me | H |
| X.45. | 2-OCF₃—Ph | CMe₂Et | Me | H |
| X.46. | 3-OCF₃—Ph | CMe₂Et | Me | H |
| X.47. | 4-OCF₃—Ph | CMe₂Et | Me | H |
| X.48. | 2-CN—Ph | CMe₂Et | Me | H |
| X.49. | 3-CN—Ph | CMe₂Et | Me | H |
| X.50. | 4-CN—Ph | CMe₂Et | Me | H |
| X.51. | 3,4-F₂—Ph | CMe₂Et | Me | H |
| X.52. | 3,4-F₂—Ph | CMe₂Et | Me | H |
| X.53. | 3,4-F₂—Ph | CMe₂Et | Me | H |
| X.54. | 3,4-Cl₂—Ph | CMe₂Et | Me | H |
| X.55. | 3,4-Cl₂—Ph | CMe₂Et | Me | H |
| X.56. | 3,4-Cl₂—Ph | CMe₂Et | Me | H |
| X.57. | Ph | CMe₂CF₃ | Me | H |
| X.58. | 2-F—Ph | CMe₂CF₃ | Me | H |
| X.59. | 3-F—Ph | CMe₂CF₃ | Me | H |
| X.60. | 4-F—Ph | CMe₂CF₃ | Me | H |
| X.61. | 2-Cl—Ph | CMe₂CF₃ | Me | H |
| X.62. | 3-Cl—Ph | CMe₂CF₃ | Me | H |
| X.63. | 4-Cl—Ph | CMe₂CF₃ | Me | H |
| X.64. | 2-Me—Ph | CMe₂CF₃ | Me | H |
| X.65. | 3-Me—Ph | CMe₂CF₃ | Me | H |
| X.66. | 4-Me—Ph | CMe₂CF₃ | Me | H |
| X.67. | 2-OMe—Ph | CMe₂CF₃ | Me | H |
| X.68. | 3-OMe—Ph | CMe₂CF₃ | Me | H |
| X.69. | 4-OMe—Ph | CMe₂CF₃ | Me | H |
| X.70. | 2-CF₃—Ph | CMe₂CF₃ | Me | H |
| X.71. | 3-CF₃—Ph | CMe₂CF₃ | Me | H |
| X.72. | 4-CF₃—Ph | CMe₂CF₃ | Me | H |
| X.73. | 2-OCF₃—Ph | CMe₂CF₃ | Me | H |
| X.74. | 3-OCF₃—Ph | CMe₂CF₃ | Me | H |
| X.75. | 4-OCF₃—Ph | CMe₂CF₃ | Me | H |
| X.76. | 2-CN—Ph | CMe₂CF₃ | Me | H |
| X.77. | 3-CN—Ph | CMe₂CF₃ | Me | H |
| X.78. | 4-CN—Ph | CMe₂CF₃ | Me | H |
| X.79. | 3,4-F₂—Ph | CMe₂CF₃ | Me | H |
| X.80. | 3,4-F₂—Ph | CMe₂CF₃ | Me | H |
| X.81. | 3,4-F₂—Ph | CMe₂CF₃ | Me | H |
| X.82. | 3,4-Cl₂—Ph | CMe₂CF₃ | Me | H |
| X.83. | 3,4-Cl₂—Ph | CMe₂CF₃ | Me | H |
| X.84. | 3,4-Cl₂—Ph | CMe₂CF₃ | Me | H |

Ph represents the phenyl group, t-Bu represents the tertiary butyl group, Me represents the methyl group, Et represents the ethyl group.

Table 1: This table discloses the 84 compounds 1.1 to 1.84 of the formula I-1, wherein R² is methyl, and R¹, R³, R⁴ and R⁵ are as defined in Table X. For example, compound No. 1.1 has the following structure:

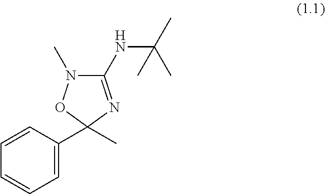 [SMILES: CN1OC(C)(c2ccccc2)N=C1NC(C)(C)C]

Table 2: This table discloses the 84 compounds 2.1 to 2.84 of the formula I-1, wherein R² is ethyl, and R¹, R³, R⁴ and R⁵ are as defined in Table X.

Table 3: This table discloses the 84 compounds 3.1 to 3.84 of the formula I-1, wherein R² is n-propyl, and R¹, R³, R⁴ and R⁵ are as defined in Table X.

Table 4: This table discloses the 84 compounds 4.1 to 4.84 of the formula I-1, wherein R² is cyclopropyl, and R¹, R³, R⁴ and R⁵ are as defined in Table X.

Table 5: This table discloses the 84 compounds 5.1 to 5.84 of the formula I-1, wherein R² is n-butyl, and R¹, R³, R⁴ and R⁵ are as defined in Table X.

Table 6: This table discloses the 84 compounds 6.1 to 6.84 of the formula I-1, wherein R² is cyclobutyl, and R¹, R³, R⁴ and R⁵ are as defined in Table X.

Table 7: This table discloses the 84 compounds 7.1 to 7.84 of the formula I-1, wherein R² is n-pentyl, and R¹, R³, R⁴ and R⁵ are as defined in Table X.

Table 8: This table discloses the 84 compounds 8.1 to 8.84 of the formula I-1, wherein R² is n-hexyl, and R¹, R³, R⁴ and R⁵ are as defined in Table X.

Table 9: This table discloses the 84 compounds 9.1 to 9.84 of the formula I-1, wherein R² is phenyl, and R¹, R³, R⁴ and R⁵ are as defined in Table X.

Table 10: This table discloses the 84 compounds 10.1 to 10.84 of the formula I-1, wherein R² is 4-F-phenyl, and R¹, R³, R⁴ and R⁵ are as defined in Table X.

Table 11: This table discloses the 84 compounds 11.1 to 11.84 of the formula I-1, wherein R² is phenethyl, and R¹, R³, R⁴ and R⁵ are as defined in Table X.

Table 12: This table discloses the 84 compounds 12.1 to 12.84 of the formula I-1, wherein R² is 3-methyloxetan-3-yl, and R¹, R³, R⁴ and R⁵ are as defined in Table X.

Table 13: This table discloses the 84 compounds 13.1 to 13.84 of the formula I-1, wherein R² is trifluoromethyl and R¹, R³, R⁴ and R⁵ are as defined in Table X.

Table 14: This table discloses the 84 compounds 14.1 to 14.84 of the formula I-1, wherein R² is 2,2,2-trifluoroethyl and R¹, R³, R⁴ and R⁵ are as defined in Table X.

Specific examples of compounds of the present invention are represented by the formula (I-2) in the following Tables 15 to 36:

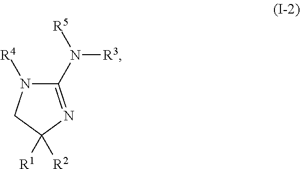

wherein R¹, R³ and R⁵ are as defined below in table Y.

Each of Tables 15 to 36, which follow the Table Y below, comprises 84 compounds of the formula (I-2) in which R¹, R³, and R⁵ have the values given in each row in Table Y, and R² and R⁴ have the values given in the relevant Tables 15 to 36. Thus compound 15. 1 corresponds to a compound of formula (I-2) where R¹, R³, and R⁵ are as defined in row 1 of Table Y and where R² and R⁴ are as defined in Table 15; compound 24.14 corresponds to a compound of formula (I-2) where R¹, R³, and R⁵ are as defined in row 14 of Table Y and where R² and R⁴ are as defined in Table 24; and so on.

TABLE Y

| Comp. No | R¹ | R³ | R⁵ |
|---|---|---|---|
| Y.1. | Ph | t-Bu | H |
| Y.2. | 2-F—Ph | t-Bu | H |
| Y.3. | 3-F—Ph | t-Bu | H |
| Y.4. | 4-F—Ph | t-Bu | H |
| Y.5. | 2-Cl—Ph | t-Bu | H |
| Y.6. | 3-Cl—Ph | t-Bu | H |
| Y.7. | 4-Cl—Ph | t-Bu | H |
| Y.8. | 2-Me—Ph | t-Bu | H |
| Y.9. | 3-Me—Ph | t-Bu | H |
| Y.10. | 4-Me—Ph | t-Bu | H |
| Y.11. | 2-OMe—Ph | t-Bu | H |
| Y.12. | 3-OMe—Ph | t-Bu | H |
| Y.13. | 4-OMe—Ph | t-Bu | H |
| Y.14. | 2-CF₃—Ph | t-Bu | H |
| Y.15. | 3-CF₃—Ph | t-Bu | H |
| Y.16. | 4-CF₃—Ph | t-Bu | H |
| Y.17. | 2-OCF₃—Ph | t-Bu | H |
| Y.18. | 3-OCF₃—Ph | t-Bu | H |
| Y.19. | 4-OCF₃—Ph | t-Bu | H |
| Y.20. | 2-CN—Ph | t-Bu | H |
| Y.21. | 3-CN—Ph | t-Bu | H |
| Y.22. | 4-CN—Ph | t-Bu | H |
| Y.23. | 3,4-F₂—Ph | t-Bu | H |
| Y.24. | 3,4-F₂—Ph | t-Bu | H |
| Y.25. | 3,4-F₂—Ph | t-Bu | H |
| Y.26. | 3,4-Cl₂—Ph | t-Bu | H |
| Y.27. | 3,4-Cl₂—Ph | t-Bu | H |
| Y.28. | 3,4-Cl₂—Ph | t-Bu | H |
| Y.29. | Ph | CMe₂Et | H |
| Y.30. | 2-F—Ph | CMe₂Et | H |
| Y.31. | 3-F—Ph | CMe₂Et | H |
| Y.32. | 4-F—Ph | CMe₂Et | H |
| Y.33. | 2-Cl—Ph | CMe₂Et | H |
| Y.34. | 3-Cl—Ph | CMe₂Et | H |
| Y.35. | 4-Cl—Ph | CMe₂Et | H |
| Y.36. | 2-Me—Ph | CMe₂Et | H |
| Y.37. | 3-Me—Ph | CMe₂Et | H |
| Y.38. | 4-Me—Ph | CMe₂Et | H |
| Y.39. | 2-OMe—Ph | CMe₂Et | H |
| Y.40. | 3-OMe—Ph | CMe₂Et | H |
| Y.41. | 4-OMe—Ph | CMe₂Et | H |
| Y.42. | 2-CF₃—Ph | CMe₂Et | H |
| Y.43. | 3-CF₃—Ph | CMe₂Et | H |
| Y.44. | 4-CF₃—Ph | CMe₂Et | H |
| Y.45. | 2-OCF₃—Ph | CMe₂Et | H |
| Y.46. | 3-OCF₃—Ph | CMe₂Et | H |
| Y.47. | 4-OCF₃—Ph | CMe₂Et | H |
| Y.48. | 2-CN—Ph | CMe₂Et | H |
| Y.49. | 3-CN—Ph | CMe₂Et | H |
| Y.50. | 4-CN—Ph | CMe₂Et | H |
| Y.51. | 3,4-F₂—Ph | CMe₂Et | H |
| Y.52. | 3,4-F₂—Ph | CMe₂Et | H |
| Y.53. | 3,4-F₂—Ph | CMe₂Et | H |
| Y.54. | 3,4-Cl₂—Ph | CMe₂Et | H |
| Y.55. | 3,4-Cl₂—Ph | CMe₂Et | H |
| Y.56. | 3,4-Cl₂—Ph | CMe₂Et | H |
| Y.57. | Ph | CMe₂CF₃ | H |
| Y.58. | 2-F—Ph | CMe₂CF₃ | H |
| Y.59. | 3-F—Ph | CMe₂CF₃ | H |
| Y.60. | 4-F—Ph | CMe₂CF₃ | H |
| Y.61. | 2-Cl—Ph | CMe₂CF₃ | H |
| Y.62. | 3-Cl—Ph | CMe₂CF₃ | H |
| Y.63. | 4-Cl—Ph | CMe₂CF₃ | H |
| Y.64. | 2-Me—Ph | CMe₂CF₃ | H |
| Y.65. | 3-Me—Ph | CMe₂CF₃ | H |
| Y.66. | 4-Me—Ph | CMe₂CF₃ | H |
| Y.67. | 2-OMe—Ph | CMe₂CF₃ | H |
| Y.68. | 3-OMe—Ph | CMe₂CF₃ | H |
| Y.69. | 4-OMe—Ph | CMe₂CF₃ | H |
| Y.70. | 2-CF₃—Ph | CMe₂CF₃ | H |
| Y.71. | 3-CF₃—Ph | CMe₂CF₃ | H |
| Y.72. | 4-CF₃—Ph | CMe₂CF₃ | H |
| Y.73. | 2-OCF₃—Ph | CMe₂CF₃ | H |
| Y.74. | 3-OCF₃—Ph | CMe₂CF₃ | H |
| Y.75. | 4-OCF₃—Ph | CMe₂CF₃ | H |
| Y.76. | 2-CN—Ph | CMe₂CF₃ | H |
| Y.77. | 3-CN—Ph | CMe₂CF₃ | H |
| Y.78. | 4-CN—Ph | CMe₂CF₃ | H |
| Y.79. | 3,4-F₂—Ph | CMe₂CF₃ | H |
| Y.80. | 3,4-F₂—Ph | CMe₂CF₃ | H |
| Y.81. | 3,4-F₂—Ph | CMe₂CF₃ | H |
| Y.82. | 3,4-Cl₂—Ph | CMe₂CF₃ | H |
| Y.83. | 3,4-Cl₂—Ph | CMe₂CF₃ | H |
| Y.84. | 3,4-Cl₂—Ph | CMe₂CF₃ | H |

Ph represents the phenyl group, t-Bu represents the tertiary butyl group, Me represents the methyl group, Et represents the ethyl group.

Table 15: This table discloses the 84 compounds 15.1 to 15.84 of the formula I-2, wherein R² is methyl, R⁴ is methyl and R¹, R³ and R⁵ are as defined in Table Y. For example, compound No. 15.1 has the following structure:

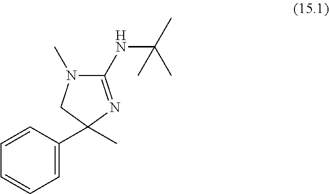 [SMILES: CN1CC(C)(c2ccccc2)N=C1NC(C)(C)C]

Table 16: This table discloses the 84 compounds 16.1 to 16.84 of the formula I-2, wherein R² is ethyl, R⁴ is methyl and R¹, R³ and R⁵ are as defined in Table Y.

Table 17: This table discloses the 84 compounds 17.1 to 17.84 of the formula I-2, wherein R² is n-propyl, R⁴ is methyl and R¹, R³ and R⁵ are as defined in Table Y.

Table 18: This table discloses the 84 compounds 18.1 to 18.84 of the formula I-2, wherein R² is cyclopropyl, R⁴ is methyl and R¹, R³ and R⁵ are as defined in Table Y.

Table 19: This table discloses the 84 compounds 19.1 to 19.84 of the formula I-2, wherein R² is n-butyl, R⁴ is methyl and R¹, R³ and R⁵ are as defined in Table Y.

Table 20: This table discloses the 84 compounds 20.1 to 20.84 of the formula I-2, wherein R² is cyclobutyl, R⁴ is methyl and R¹, R³ and R⁵ are as defined in Table Y.

Table 21: This table discloses the 84 compounds 21.1 to 21.84 of the formula I-2, wherein R² is n-pentyl, R⁴ is methyl and R¹, R³ and R⁵ are as defined in Table Y.

Table 22: This table discloses the 84 compounds 22.1 to 22.84 of the formula I-2, wherein R² is n-hexyl, R⁴ is methyl and R¹, R³ and R⁵ are as defined in Table Y.

Table 23: This table discloses the 84 compounds 23.1 to 23.84 of the formula I-2, wherein R² is methyl, R⁴ is methoxy and R¹, R³ and R⁵ are as defined in Table Y.

Table 24: This table discloses the 84 compounds 24.1 to 24.84 of the formula I-2, wherein R² is ethyl, R⁴ is methoxy and R¹, R³ and R⁵ are as defined in Table Y.

Table 25: This table discloses the 84 compounds 25.1 to 25.84 of the formula I-2, wherein R² is n-propyl, R⁴ is methoxy and R¹, R³ and R⁵ are as defined in Table Y.

Table 26: This table discloses the 84 compounds 26.1 to 26.84 of the formula I-2, wherein R² is cyclopropyl, R⁴ is methoxy and R¹, R³ and R⁵ are as defined in Table Y.

Table 27: This table discloses the 84 compounds 27.1 to 27.84 of the formula I-2, wherein R² is n-butyl, R⁴ is methoxy and R¹, R³ and R⁵ are as defined in Table Y.

Table 28: This table discloses the 84 compounds 28.1 to 28.84 of the formula I-2, wherein R² is cyclobutyl, R⁴ is methoxy and R¹, R³ and R⁵ are as defined in Table Y.

Table 29: This table discloses the 84 compounds 29.1 to 29.84 of the formula I-2, wherein R² is n-pentyl, R⁴ is methoxy and R¹, R³ and R⁵ are as defined in Table Y.

Table 30: This table discloses the 84 compounds 30.1 to 30.84 of the formula I-2, wherein R² is n-hexyl, R⁴ is methoxy and R¹, R³ and R⁵ are as defined in Table Y.

Table 31: This table discloses the 84 compounds 31.1 to 31.84 of the formula I-1, wherein R² is phenyl, R⁴ is methyl and R¹, R³ and R⁵ are as defined in Table Y.

Table 32: This table discloses the 84 compounds 32.1 to 32.84 of the formula I-1, wherein R² is 4-F-phenyl, R⁴ is methyl and R¹, R³ and R⁵ are as defined in Table Y.

Table 33: This table discloses the 84 compounds 33.1 to 33.84 of the formula I-1, wherein R² is phenethyl, R⁴ is methyl and R¹, R³ and R⁵ are as defined in Table Y.

Table 34: This table discloses the 84 compounds 34.1 to 34.84 of the formula I-1, wherein R² is 3-methyloxetan-3-yl, R⁴ is methyl and R¹, R³ and R⁵ are as defined in Table Y.

Table 35: This table discloses the 84 compounds 35.1 to 35.84 of the formula I-1, wherein R² is trifluoromethyl, R⁴ is methyl and R¹, R³ and R⁵ are as defined in Table Y.

Table 36: This table discloses the 84 compounds 36.1 to 36.84 of the formula I-1, wherein R² is 2,2,2-trifluoroethyl, R⁴ is methyl and R¹, R³ and R⁵ are as defined in Table Y.

If appropriate, agrochemically acceptable salt, stereoisomer, enantiomer, tautomer and/or N-oxide of each of the compounds in tables 1 to 36 is also disclosed.

Compounds of formula I-1 in which R¹, R², R³, R⁴ and R⁵ are defined as above in structure I, except that R⁵ is not hydrogen, may be prepared (as shown in scheme 1) by reacting compounds of formula I-1 in which R¹, R², R³, and R⁴ are defined as above in structure I and R⁵ is hydrogen with a compound R⁵-LG₁ in which R⁵ is defined as above in structure I, except that R⁵ is not hydrogen in the presence of a base. Bases may be metal hydrides, such as sodium hydride, potassium hydride or calcium hydride or metal alkoxides, such as sodium methoxide or potassium t-butoxide, or organometals, such as methyllithium, butyllithium, alkylmagnesium halide, or a basic salt, such as potassium carbonate. A solvent can be used. The solvent could be, for example, a polar aprotic solvent like dimethyl formamide (DMF) or acetonitrile or an ether like tetrahydrofuran (THF) or dimethoxyethane. The reaction may be performed between 0° C. and 80° C., but preferably in DMF between 0° C. and 25° C. LG₁ is a leaving group, such as bromide, chloride, iodide, mesylate, triflate, tosylate and the like.

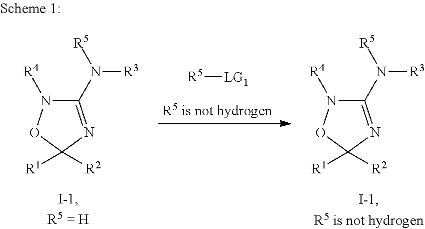

Compounds of formula I-1 in which R¹, R², R³, and R⁴ are defined as above in structure I and R⁵ is hydrogen may be prepared (as shown in scheme 2) by reacting N-hydroxy-guanidine compounds of formula II in which R¹, R², R³, and R⁴ are defined as above in structure I and R⁵ is hydrogen with an oxidizing reagent, for example MnO₂, NaOCl, Ag₂O, CuO, Cu₂O, air/TEMPO. The reaction may be performed in halogenated solvents, for example CH₂Cl₂, ClCH₂CH₂Cl, CHCl₃, preferably CH₂Cl₂, in ethers, for examples in THF or in DMF. The reaction may be performed between 0° C. and 60° C., preferably between 0° C. and 30° C.

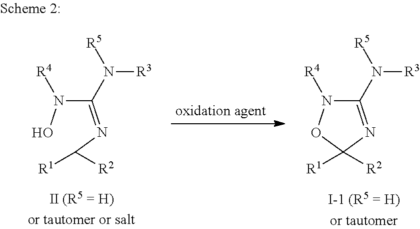

Alternatively compounds of formula I-1 in which R¹, R², R³, and R⁴ are defined as above in structure I and R⁵ is hydrogen may be prepared (as shown in scheme 3) by reacting N-hydroxy-guanidine compounds of formula II in which R¹, R², R³, and R⁴ are defined as above in structure I and R⁵ is hydrogen with a halogenating agent, such as N-chlorosuccinimide, N-bromosuccinimide, Br₂, Cl₂ in the presence of a base or followed by addition of a base, such as pyridine or an alkylamine, for example triethylamine, or a basic salt, such as potassium carbonate. The reaction may be performed in halogenated solvents, such as CH₂Cl₂, ClCH₂CH₂Cl, CHCl₃, preferably CH₂Cl₂. The reaction may be performed between 0° C. and 60° C.

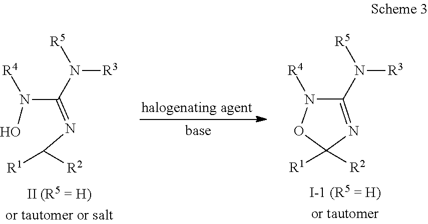

N-hydroxy-guanidine compounds of formula II in which R¹, R², R³, and R⁴ are defined as above in structure I and R⁵ is hydrogen may be prepared (as shown in scheme 4) by reaction of carbodiimide compounds of formula III in which R¹, R², and R³ are defined as above in structure I with a hydroxylamine R⁴—NH—OH in which R⁴ is defined as above in structure I. A solvent can be used. The preferred solvents are alcohols, such as MeOH or EtOH. A base might be added. The base could be pyridine, an alkylamine, for example triethylamine, or a basic salt, such as potassium carbonate. The reaction may be performed between 0° C. and 100° C., preferably between 0° C. and 30° C.

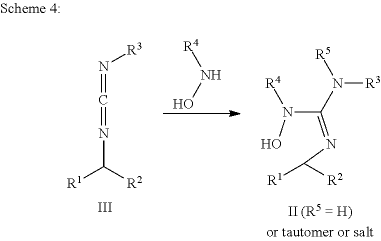

Carbodiimide compounds of formula III in which R¹, R², and R³ are defined as above in structure I may be prepared (as shown in scheme 5) by reacting thiourea compounds of formula IV in which R¹, R², and R³ are defined as above in structure I with bis(2-pyridyloxy)methanethione in the presence of a base catalyst, for example N,N-dimethyl-4-amino-pyridine. A solvent can be used. The solvent may be a halogenated solvent, such as CH₂Cl₂, ClCH₂CH₂Cl, CHC₃, preferably CH₂Cl₂ or DMF or acetonitrile. The reaction may be performed between 0° C. and 100° C., preferably at 80° C.

Alternatively carbodiimide compounds of formula III in which R¹, R², and R³ are defined as above in structure I may be prepared (as shown in scheme 5) by reacting thiourea compounds of formula IV in which R¹, R², and R³ are defined as above in structure I with 2-chloro-1,3-dimethyl-4,5-dihydroimidazol-1-ium chloride in the presence of a base. The base could be pyridine, an alkylamine, for example triethylamine, or a basic salt, such as potassium carbonate. A solvent can be used. The solvent may be a halogenated solvent, such as CH₂Cl₂, ClCH₂CH₂Cl, CHC₃, preferably CH₂Cl₂ or DMF or acetonitrile. The reaction may be performed between 0° C. and 100° C., preferably between 0° C. and 30° C.

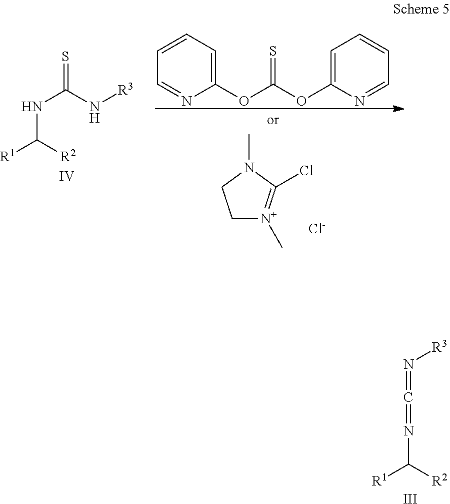

Alternatively carbodiimide compounds of formula III in which R¹, R², and R³ are defined as above in structure I may be prepared (as shown in scheme 6) by reacting urea compounds of formula V in which R¹, R², and R³ are defined as above in structure I CBr₄ and triphenylphospine in the presence of a base. The base could be pyridine, an alkylamine, for example triethylamine, or a basic salt, such as potassium carbonate. A solvent can be used. The solvent may be a halogenated solvent, such as CH₂Cl₂, ClCH₂CH₂Cl, CHCl₃, preferably CH₂Cl₂. The reaction may be performed between 0° C. and 50° C., preferably between 0° C. and 30° C.

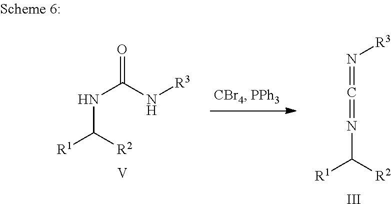

Thiourea compounds of formula IV in which R¹, R², and R³ are defined as above in structure I may be prepared (as shown in scheme 7) by reacting isothiocyanate compounds of formula VI in which R¹ and R² are defined as above in structure I with amines H₂N—R³ in which R³ is defined as above in structure I. A solvent can be used. The solvent may be a halogenated solvent, such as CH₂Cl₂, ClCH₂CH₂Cl, CHCl₃, preferably CH₂Cl₂. The reaction may be performed between 0° C. and 50° C., preferably between 0° C. and 30° C.

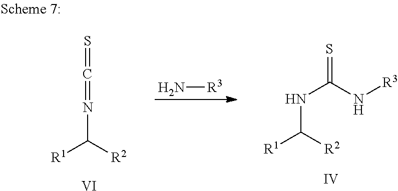

Alternatively thiourea compounds of formula IV in which R¹, R², and R³ are defined as above in structure I may be prepared (as shown in scheme 8) by reacting amines of formula VII in which R¹ and R² are defined as above in structure I with isothiocyanates S═C═N—R³ in which R³ is defined as above in structure I. A solvent can be used. The solvent may be a halogenated solvent, such as CH₂Cl₂, ClCH₂CH₂Cl, CHCl₃, preferably CH₂Cl₂. The reaction may be performed between 0° C. and 50° C., preferably between 0° C. and 30° C.

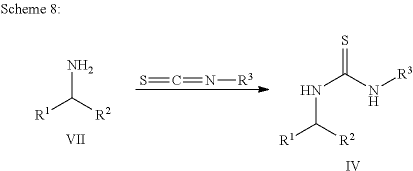

Urea compounds of formula V in which R¹, R², and R³ are defined as above in structure I may be prepared (as shown in scheme 9) by reacting isocyanate compounds of formula VIII in which R¹ and R² are defined as above in structure I with amines H₂N—R³ in which R³ is defined as above in structure I. A solvent can be used. The solvent may be a halogenated solvent, such as CH₂Cl₂, ClCH₂CH₂Cl, CHCl₃, preferably CH₂Cl₂. The reaction may be performed between 0° C. and 50° C., preferably between 0° C. and 30° C.

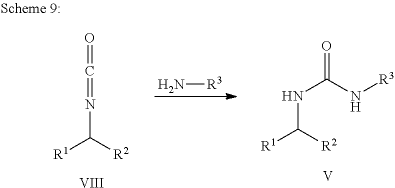

Alternatively urea compounds of formula V in which R¹, R², and R³ are defined as above in structure I may be prepared (as shown in scheme 10) by reacting amines of formula VII in which R¹ and R² are defined as above in structure I with isocyanates O═C═N—R³ in which R³ is defined as above in structure I. A solvent can be used. The solvent may be a halogenated solvent, such as CH₂Cl₂, ClCH₂CH₂Cl, CHCl₃, preferably CH₂Cl₂. The reaction may be performed between 0° C. and 50° C., preferably between 0° C. and 30° C.

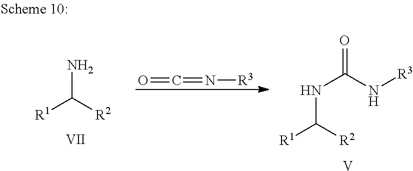

Isothiocyanate compounds of formula VI in which R1 and R2 are defined as above in structure I may be prepared (as shown in scheme 11) by reacting amines of formula VII in which R¹ and R² are defined as above in structure I with thiophosgene in the presence of a base. The base could be could be pyridine, an alkylamine, for example triethylamine, or a basic salt, such as potassium hydrogen carbonate or potassium carbonate. A solvent can be used. The solvent may be a halogenated solvent, such as CH₂Cl₂, ClCH₂CH₂Cl, CHCl₃, preferably CH₂Cl₂. The reaction may be performed between 0° C. and 50° C., preferably between 0° C. and 30° C.

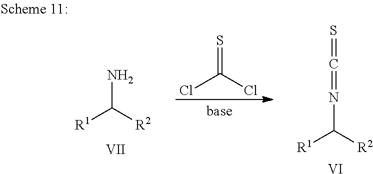

Isocyanate compounds of formula VI in which R¹ and R² are defined as above in structure I may be prepared (as shown in scheme 12) by reacting amines of formula VII in which R¹ and R² are defined as above in structure I with phosgene or triphosgene in the presence of a base. The base could be could be pyridine, an alkylamine, for example triethylamine, or a basic salt, such as potassium hydrogen carbonate or potassium carbonate. A solvent can be used. The solvent may be a halogenated solvent, such as CH₂Cl₂, ClCH₂CH₂Cl, CHCl₃, preferably CH₂Cl₂. The reaction may be performed between 0° C. and 50° C., preferably between 0° C. and 30° C.

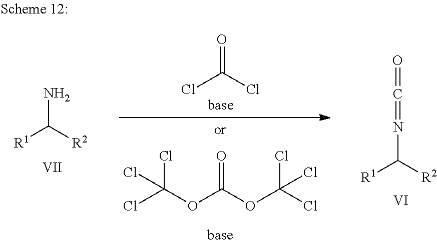

Amines of structure VII in which R¹ and R² are defined as above in structure I are known compounds or can be prepared by known procedures (Scheme 13), for example by reductive amination of ketones of structure IX in which R¹ and R² are defined as above in structure I or reduction of oximes of structure X in which R¹ and R² are defined as above in structure I and which are prepared by known procedures from ketones IX. The ketones IX are known compounds or can be prepared by known procedures.

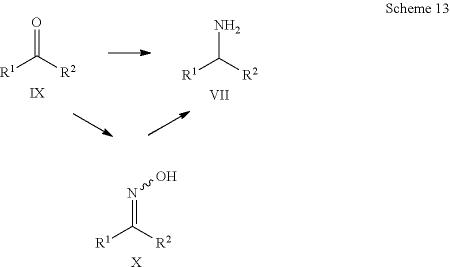

Alternatively compounds of formula I-1 in which R¹, R² and R⁴ are defined as above in structure I, R⁴ is t-butyl and R⁵ is hydrogen may be prepared (as shown in scheme 14) by reacting compounds of formula I-1 in which R¹, R² and R⁴ are defined as above in structure I and R⁴ and R⁵ are hydrogen with an alkylating agent LG₂-t-butyl. LG₂ is leaving group. The reaction may be performed in halogenated solvents, for example CH₂Cl₂, ClCH₂CH₂Cl, CHCl₃, preferably CH₂Cl₂, in ethers, for examples in THF or in hexane. The reaction may be catalyzed by an acid, for example a Lewis acid such as BF₃-Et₂O. The reaction may be performed between −40° C. and 60° C., preferably between 0° C. and 30° C.

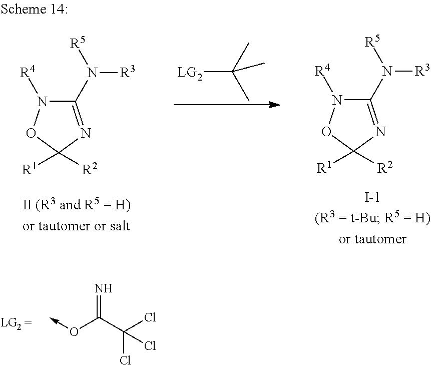

Compounds of formula I-1 in which R¹, R² and R⁴ are defined as above in structure I and R⁴ and R⁵ are hydrogen may be prepared (as shown in scheme 15) by reacting cyano-imine compounds of formula XI in which R¹ and R² are defined as above in structure I with hydroxylamines HO—NH—R⁴ in which R⁴ is defined as above in structure I in the presence of a base. The base could be pyridine, an alkylamine, for example triethylamine, or a basic salt, such as potassium carbonate. A solvent can be used. The solvent may be a halogenated solvent, such as CH₂Cl₂, ClCH₂CH₂Cl, CHCl₃, preferably CH₂Cl₂ or an ether, for example THF. Addition of molecular sieves may increase the yield. The reaction may be performed between 0° C. and 80° C., preferably between 0° C. and 30° C.

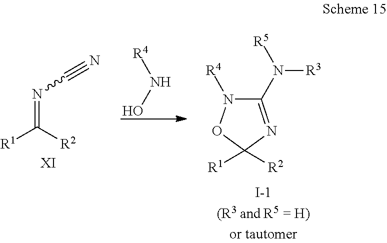

Cyano-imine compounds of formula XI in which R¹ and R² are defined as above in structure I may be prepared (as shown in scheme 16) by reacting ketones of structure IX in which R¹ and R² are defined as above in structure I with N,N′-bis(trimethylsilyl)methanediimine. The reaction can be performed in halogenated solvents, for example CH₂Cl₂, ClCH₂CH₂Cl, CHCl₃, preferably CH₂Cl₂, in ethers, for examples in THF or in hexane. The reaction may be catalyzed by an acid, for example a Lewis acid such as titan tetrachloride. The reaction may be performed between −40° C. and 60° C., preferably between 0° C. and 30° C.

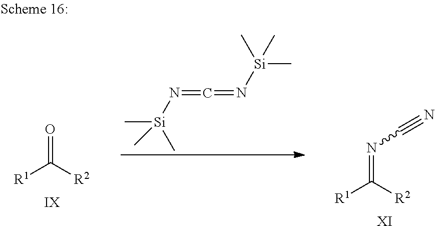

The ketones IX are known compounds or can be prepared by known procedures. N,N-bis(trimethylsilyl)methanediimine can be prepared from cyanamide and trimethylsilyl chloride according to Birkofer, L. et al. Tetrahedron Lett., 1962, 195-198. Alternatively N,N′-bis(trimethylsilyl)methanediimine can be prepared from cyanamide and trimethylsilyl cyanide according to Mai, K. and Patil, G., J. Org. Chem. 1987, 52(2), 275-276.

Compounds of formula I-2 in which R¹, R², R³, R⁴ and R⁵ are defined as above in structure I may be prepared (as shown in scheme 17) by reduction of compounds of formula XII in which R¹, R², R³, R⁴ and R⁵ are defined as above in structure I. The reducing reagent could contain a metalhydride, such as AlH₃, LiAlH₄, LiAlH₄/AlCl₃, t-Bu₂—AlH, LiHAl(OMe)₃, BH₃, BH₃—SMe₂, NaBH₄/HOAc, NaBH₄/AICl₃. A solvent can be used. For certain reagents the solvent could be, for example, a polar protic solvent like an alcohol, such as MeOH or EtOH or an acid such as acetic acid. For other reagents an ether like THF or diethyl ether could be preferred. The reaction may be performed between 0° C. and 80° C., but preferably between 0° C. and 25° C.

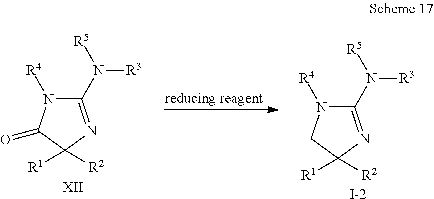

Compounds of formula XII in which R¹, R², R³, R⁴ and R⁵ are defined as above in structure I may be prepared (as shown in scheme 18) by reaction of compounds of formula XIII in which R¹, R² and R⁴ are defined as above in structure I with amines HNR³R⁵ in which R³ and R⁵ are defined as above in structure I in the presence of silver nitrate and a basic salt, such as potassium carbonate. The reaction may be performed between 100° C. and 150° C., but preferably at 135° C. under microwave irradiation.

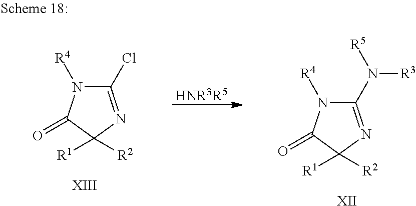

Compounds of formula XIII in which R¹, R², R³ and R⁴ are defined as above in structure I may be prepared (as shown in scheme 19) by reaction of compounds of formula XIV in which R¹, R² and R⁴ are defined as above in structure I with sulfurylchloride. The reaction may be performed in halogenated solvents, for example CH₂Cl₂, ClCH₂CH₂Cl, CHCl₃, preferably CH₂Cl₂, The reaction may be performed between 0° C. and 40° C., but preferably between 0° C. and 30° C.

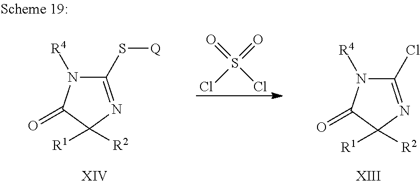

Compounds of formula XIV in which R¹, R², R³ and R⁴ are defined as above in structure I may be prepared (as shown in scheme 20) by reaction of compounds of formula XV in which R¹, R² and R⁴ are defined as above in structure I with Q-LG₃ in the presence of a base. Q may be C₁-C₄-alkyl or benzyl. LG₃ is a leaving group, such as bromide, chloride, iodide, mesylate, triflate, tosylate and the like. The base may be a basic salt, such as potassium carbonate. A solvent can be used. The solvent could be, for example, a polar aprotic solvent like DMF or acetonitrile or an ether like THF or diethyl ether. The reaction may be performed between 0° C. and 80° C., but preferably between 50° C. and 70° C.

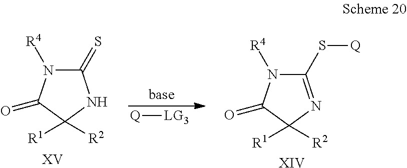

Compounds of formula XV in which R¹, R² and R⁴ and R⁵ are defined as above in structure I may be prepared (as shown in scheme 21) by reaction of compounds of formula XVI in which R¹, R² and R⁴ are defined as above in structure I with Lawessons's reagent. A solvent can be used. The solvent could be an aromatic solvent such as toluene. The reaction may be performed between 80° C. and 150° C., but preferably at 120° C.

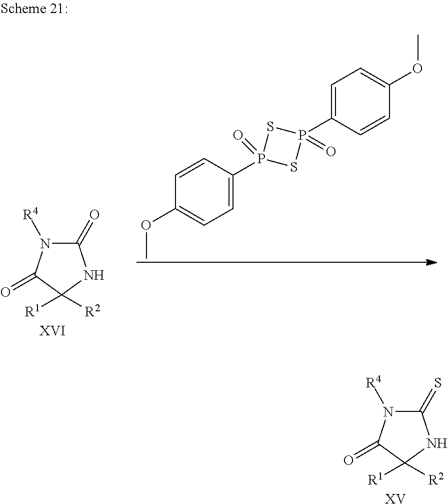

Compounds of formula XVI in which R¹, R² and R⁴ are defined as above in structure I may be prepared (as shown in scheme 22) by reaction of hydantoins of formula XVII in which R¹ and R² are defined as above in structure I with R⁴-LG₄ in which R⁴ is defined as above in structure I in the presence of a base. Bases may be metal hydrides, such as sodium hydride, potassium hydride or calcium hydride or metal alkoxides, such as sodium methoxide or potassium t-butoxide, or organometals, such as methyllithium, butyllithium, alkylmagnesium halide, or a basic salt, such as potassium carbonate. A solvent can be used. The solvent could be, for example, a polar aprotic solvent like DMF or acetonitrile or an ether like THF or dimethoxyethane. The reaction may be performed between 0° C. and 80° C., but preferably in THF between 0° C. and 30° C. LG₄ is a leaving group, such as bromide, chloride, iodide, mesylate, triflate, tosylate and the like.

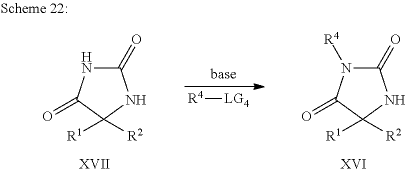

Compounds of formula XVII in which R¹, R² and R⁴ are defined as above in structure I may be prepared (as shown in scheme 22) by reaction of ketones of formula IX in which R¹ and R² are defined as above in structure I with potassium cyanide and ammonium carbonate. A solvent can be used. The solvent could be, for example, a polar protic solvent like water or an alcohol, for example methanol or ethanol or a mixture of an alcohol and water. The reaction may be performed between 80° C. and 150° C., but preferably between 80° C. and 100° C. in a sealed tube. Scheme 22:

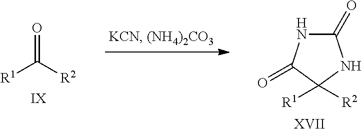

The ketones IX in which R¹ and R² are defined as above in structure I are known compounds or can be prepared by known procedures.

Alternatively compounds of formula XV in which R¹, R² and R⁴ are defined as above in structure I may be prepared (as shown in scheme 23) by reaction of diketones of formula XVIII in which R¹ and R² are defined as above in structure I with thiourea compound of formula XIX in which R⁴ is defined as above in structure I.

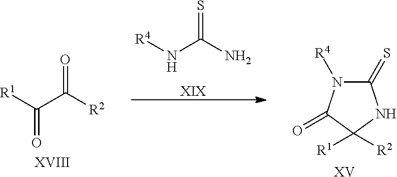

The diketones XVIII in which R¹ and R² are defined as above in structure I are known compounds or can be prepared by known procedures.

Alternatively compounds of structure XVII in which R², R³, R⁴ and R⁵ are defined as above in structure I and X is alkyl, cycloalkyl or phenyl or an optionally substituted phenyl may be prepared by reacting compound XVI in which R², R³, R⁴ and R⁵ are defined as above in structure I and LG₅ is chlorine, bromine or iodine, or an aryl- or alkylsulfonate such as trifluoromethanesulfonate, or any other similar leaving group, with a reagent LG₆-X under palladium catalysis in which LG₆ may be a boron-derived functional group, as for example B(OH)₂ and X is alkyl, cycloalkyl or phenyl or an optionally substituted phenyl. The reaction can be catalyzed by a palladium based catalyst, for example tetrakis(triphenylphosphine)-palladium or (1,1′bis(diphenylphosphino)-ferrocene)dichloropalladium-dichloromethane (1:1 complex), in presence of a base, like sodium carbonate or cesium fluoride, in a solvent or a solvent mixture, like, for example a mixture of 1,2-dimethoxyethane and water or of dioxane and water, preferably under inert atmosphere. The reaction temperature can preferentially range from room temperature to the boiling point of the reaction mixture.

Compounds of structure XVII in which R², R³, R⁴ and R⁵ are defined as above in structure I and X is CN may be prepared by reacting compound XVI in which R², R³, R⁴ and R⁵ are defined as above in structure I and LG₅ is chlorine, bromine or iodine, or an aryl- or alkylsulfonate such as trifluoromethanesulfonate, or any other similar leaving group, with a Zn reagent, such as Zn(CN)₂.

The reaction can be catalyzed by a palladium based catalyst, for example tetrakis(triphenylphosphine)-palladium or (1,1′bis(diphenylphosphino)-ferrocene)dichloropalladium-dichloromethane (1:1 complex), in presence of a base, like sodium carbonate or cesium fluoride, in a solvent or a solvent mixture, like, for example a mixture of DMF or toluene or toluene-water, preferably under inert atmosphere.

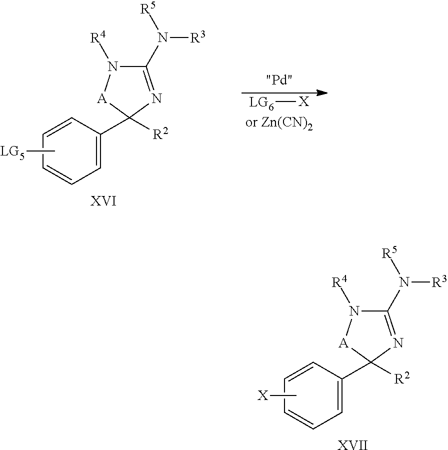

Protecting Plants

Certain compounds defined by of any one of the formulae (I-2) and (I-A1) have been found to control the damage caused by a pest, specifically insect pests, in particular damage on plants, such as crops. Such compounds are herein referred to as a COMPOUND Q.

In an embodiment, Compound Q is any one of the formulae (I-2) and (I-A1) (groups A to G).

In an embodiment, Compound Q is represented by the compounds of formula (I-A1), wherein R¹ is phenyl substituted by 1 to 3 substituents independently selected from C₁-C₄-alkyl, C₁-C₄-alkoxy, C₁-C₄-haloalkoxy, C₁-C₄-haloalkyl, halogen and CN; R² is n-propyl or n-butyl or n-pentyl; R³ is t-butyl, C(CH₃)₂(C₂H₅), or C(CH₃)₂(CF₃); R⁴ is methyl; and R⁵ is H. More preferably Compound Q is represented by the compounds of formula (I-A1), wherein R¹ is 2-fluoro phenyl; R² is cyclopropyl; R³ is t-butyl, C(CH₃)₂(C₂H₅) or C(CH₃)₂(CF₃); R⁴ is methyl; and R⁵ is H; or R¹ is 4-fluoro phenyl; R² is cyclopropyl; R³ is C(CH₃)₂(C₂H₅), or C(CH₃)₂(CF₃); R⁴ is methyl; and R⁵ is H; or R¹ is aryl substituted by 1 to 3 substituents independently selected from C₁-C₄-alkyl, C₁-C₄-alkoxy, C₁-C₄-haloalkoxy, C₁-C₄-haloalkyl, halogen and CN; R² is n-propyl or n-butyl; R³ is C₁-C₆-alkyl, or C₁-C₆-haloalkyl; R⁴ is C₁-C₄-alkyl; and R⁵ is H.

A COMPOUND Q can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fiber products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

Examples of pest species which may be controlled by the compounds of formula (I) include: Myzus persicae (aphid), Aphis gossypii (aphid), Aphis fabae (aphid), Lygus spp. (capsids), Dysdercus spp. (capsids), Nilaparvata lugens (planthopper), Nephotettix cincticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulfureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp. (citrus nematodes), Haemonchus contortus (barber pole worm), Caenorhabditis elegans (vinegar eelworm), Trichostrongylus spp. (gastro intestinal nematodes) and Deroceras reticulatum (slug).

The invention therefore provides a method of controlling insects, acarines, nematodes or molluscs which comprises applying an insecticidally, acaricidally, nematicidally or molluscicidally effective amount of a COMPOUND Q or a composition containing a COMPOUND Q, to a pest, a locus of pest, preferably a plant, to a plant susceptible to attack by a pest or to plant propagation material thereof, such as a seed. The compounds of formula (I) are preferably used against insects or acarines.

The term “plant” as used herein includes seedlings, bushes and trees.

Suitable target plants or crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family, latex plants and ornamentals.

Crops are to be understood as also including those crops which have been rendered tolerant to herbicides or classes of herbicides (e.g., ALS-, GS-, EPSPS-, PPO- and HPPD-inhibitors) by conventional methods of breeding or by genetic engineering. An example of a crop that has been rendered tolerant to imidazolinones, e.g., imazamox, by conventional methods of breeding is Clearfield® summer rape (canola). Examples of crops that have been rendered tolerant to herbicides by genetic engineering methods include, e.g., glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®.

Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g., improved storage stability, higher nutritional value and improved flavor).

In order to apply a COMPOUND Q as an insecticide, acaricide, nematicide or molluscicide to a pest, a locus of pest, to a plant susceptible to attack by a pest, or propagation material thereof, the COMPOUND Q is usually formulated into a composition which includes, in addition to the compound of formula (I), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a COMPOUND Q. The composition is generally used for the control of pests such that a COMPOUND Q is applied at a rate of from 0.1 g to 10 kg per hectare, preferably from 1 g to 6 kg per hectare, more preferably from 1 g to 1 kg per hectare.

When used in a seed dressing, a COMPOUND Q is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal, acaricidal, nematicidal or molluscicidal composition comprising an insecticidally, acaricidally, nematicidally or molluscicidally effective amount of a COMPOUND Q and a suitable carrier or diluent therefor. The composition is preferably an insecticidal or acaricidal composition.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the COMPOUND Q.

A composition may include one or more additives to improve the biological performance of the composition (for example by improving wetting, retention or distribution on surfaces; resistance to rain on treated surfaces; or uptake or mobility of the COMPOUND Q). Such additives include surface active agents, spray additives based on oils, for example certain mineral oils or natural plant oils (such as soy bean and rape seed oil), and blends of these with other bio-enhancing adjuvants (ingredients which may aid or modify the action of the COMPOUND Q).

A COMPOUND Q may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

A COMPOUND Q may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A COMPOUND Q may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of the COMPOUND Q (for example 0.0001 to 10%, by weight) depending upon the purpose for which they are to be used.

A COMPOUND Q) may be used in mixtures with fertilizers (for example nitrogen-, potassium- or phosphorus-containing fertilizers). Suitable formulation types include granules of fertilizer. The mixtures preferably contain up to 25% by weight of the COMPOUND Q.

The invention therefore also provides a fertilizer composition comprising a fertilizer and a COMPOUND Q.

The compositions of this invention may contain other compounds having biological activity, for example micronutrients or compounds having fungicidal activity or which possess plant growth regulating, herbicidal, insecticidal, nematicidal or acaricidal activity.

The COMPOUND Q may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergize the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, S-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;

b) Organophosphates, such as profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;

c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;

d) Benzoyl ureas, such as diflubenzuron, triflumuron, hexaflumuron, flufenoxuron or chlorfluazuron; e) Organic tin compounds, such as cyhexatin, fenbutatin oxide or azocyclotin;

f) Pyrazoles, such as tebufenpyrad and fenpyroximate;

g) Macrolides, such as avermectins or milbemycins, for example abamectin, emamectin benzoate, ivermectin, milbemycin, spinosad, azadirachtin or spinetoram;

h) Hormones or pheromones;

i) Organochlorine compounds, such as endosulfan (in particular alpha-endosulfan), benzene hexachloride, DDT, chlordane or dieldrin;

j) Amidines, such as chlordimeform or amitraz;

k) Fumigant agents, such as chloropicrin, dichloropropane, methyl bromide or metam;

l) Neonicotinoid compounds, such as imidacloprid, thiacloprid, acetamiprid, nitenpyram, dinotefuran, thiamethoxam, clothianidin, nithiazine or flonicamid;

m) Diacylhydrazines, such as tebufenozide, chromafenozide or methoxyfenozide;

n) Diphenyl ethers, such as diofenolan or pyriproxifen;

o) Indoxacarb;

p) Chlorfenapyr;

q) Pymetrozine;

r) Spirotetramat, spirodiclofen or spiromesifen;

s) Diamides, such as flubendiamide, chlorantraniliprole or cyantraniliprole;

t) Sulfoxaflor;

u) Metaflumizone;

v) Fipronil and Ethiprole; or

w) Pyrifluqinazon;

x) buprofezin; or

y) 4-[(6-Chloro-pyridin-3-ylmethyl)-(2,2-difluoro-ethyl)-amino]-5H-furan-2-one (DE 102006015467).

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid),

3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704), alanycarb, aldimorph, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)—N-benzyl-N-([methyl(methyl-thioethylideneaminooxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluoroimide, fluquinconazole, flusilazole, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, man-cozeb, maneb, mefenoxam, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, phenazin oxide, phosetyl-Al, phosphorus acids, phthalide, picoxystrobin (ZA1963), polyoxin D, polyram, probenazole, prochloraz, procymidone, propamocarb, propiconazole, propineb, propionic acid, pyrazophos, pyrifenox, pyrimethanil, pyroquilon, pyroxyfur, pyrrolnitrin, quaternary ammonium compounds, quinomethionate, quinoxyfen, quintozene, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb, ziram; 1,3-Dimethyl-1H-pyrazole-4-carboxylic acid (4′-methylsulfanyl-biphenyl-2-yl)-amide, 1,3-Dimethyl-1H-pyrazole-4-carboxylic acid (2-dichloromethylene-3-ethyl-1-methyl-indan-4-yl)-amide, and 1,3-Dimethyl-4H-pyrazole-4-carboxylic acid [2-(2,4-dichloro-phenyl)-2-methoxy-1-methyl-ethyl]-amide.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The compounds of the invention are also useful in the field of animal health, e.g., they may be used against parasitic invertebrate pests, more preferably against parasitic invertebrate pests in or on an animal. Examples of pests include nematodes, trematodes, cestodes, flies, mites, tricks, lice, fleas, true bugs and maggots. The animal may be a non-human animal, e.g., an animal associated with agriculture, e.g. a cow, a pig, a sheep, a goat, a horse, or a donkey, or a companion animal, e.g., a dog or a cat.

In a further aspect the invention provides a compound of the invention for use in a method of therapeutic treatment.

In a further aspect the invention relates to a method of controlling parasitic invertebrate pests in or on an animal comprising administering a pesticidally effective amount of a compound of the invention. The administration may be for example oral administration, parenteral administration or external administration, e.g., to the surface of the animal body. In a further aspect the invention relates to a compound of the invention for controlling parasitic invertebrate pests in or on an animal. In a further aspect the invention relates to use of a compound of the invention in the manufacture of a medicament for controlling parasitic invertebrate pests in or on an animal

In a further aspect, the invention relates to a method of controlling parasitic invertebrate pests comprising administering a pesticidally effective amount of a compound of the invention to the environment in which an animal resides.

In a further aspect the invention relates to a method of protecting an animal from a parasitic invertebrate pest comprising administering to the animal a pesticidally effective amount of a compound of the invention. In a further aspect the invention relates to a compound of the invention for use in protecting an animal from a parasitic invertebrate pest. In a further aspect the invention relates to use of a compound of the invention in the manufacture of a medicament for protecting an animal from a parasitic invertebrate pest.

In a further aspect the invention provides a method of treating an animal suffering from a parasitic invertebrate pest comprising administering to the animal a pesticidally effective amount of a compound of the invention. In a further aspect the invention relates to a compound of the invention for use in treating an animal suffering from a parasitic invertebrate pest. In a further aspect the invention relates to use of a compound of the invention in the manufacture of a medicament for treating an animal suffering from a parasitic invertebrate pest.

In a further aspect, the invention provides a pharmaceutical composition comprising a compound of the invention and a pharmaceutically suitable excipient.

Individual active substances can occur in more than one group or class, and at more than one place within a group or class: information about the active substances, their spectrum, sources and classifications can be found from Compendium of Pesticide Common Names (see http://www.alanwood.net/pesticides/index.html) or from the Pesticide Manual created by the British Crop Production Counci (see http://bcpcdata.com/pesticide-manual.html).

The compounds according to the invention are preventively and/or curatively valuable active ingredients in the field of pest control, even at low rates of application, which have a favorable biocidal spectrum and are well tolerated by warm-blooded species, fish and plants. Compounds Q may act against all or only individual developmental stages of normally sensitive, but also resistant, animal pests, such as insects or representatives of the order Acarina. The insecticidal or acaricidal activity of the compounds can manifest itself directly, i.e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

### Mosquito Vector Control

Mosquito-control operations are targeted against three different problems:
1. Nuisance mosquitoes bother people around homes or in parks and recreational areas;
2. Economically important mosquitoes reduce real estate values, adversely affect tourism and related business interests, or negatively impact livestock or poultry production;
3. Public health is the focus when mosquitoes are vectors, or transmitters, of infectious disease.

Many infectious diseases (e.g., malaria, dengue and yellow fever, lymphatic filariasis, and leishmaniasis) that are responsible for debilitating or even killing humans and animals in many countries, especially in tropical countries, are transmitted by insect vectors. For example, the mosquito parasite, Plasmodium falciparum, accounts for greater than 25 percent of childhood mortality outside the neonatal period. In certain parts of Africa, malaria has been ranked first by the World Bank in terms of disability-adjusted life-years lost. A number of drugs are available to treat and/or prevent some insect-borne diseases. However, not all diseases transmitted by mosquitoes can be treated efficiently. For example, there is currently no chemotherapeutic drug or vaccine available against the Dengue virus. Furthermore, in the case of antimalarial drugs, treatment with the drugs currently available is becoming less effective due to increased resistance in some Plasmodium strains. Plasmodium enters the human bloodstream as a consequence of the insect bite and causes malaria. Therefore, one of the most effective ways to prevent mosquito vector-borne illnesses is by decreasing mosquito populations in areas of high pathogen transmission and/or preventing mosquito bites in the first place. More recently, efforts have been concentrated on controlling the transmitting mosquitoes.

The three medically important genera of insects which transmit diseases are the mosquitoes Anopheles, Culex and Aedes. The genera Culex and Aedes belong to the sub-family Culicinae, while the Anopheles belongs to the sub-family Anophelinae.

Examples of diseases or pathogens transferred by the key mosquitoes are:
Anopheles: malaria, filariasis;
Culex: Japanese encephalitis, other viral diseases, filariasis; and
Aedes: yellow fever, dengue fever, chikungunya, other viral diseases (e.g., Zika virus), and filariasis;

In an attempt to reduce the problems associated with disease-transmitting mosquitoes, a wide range of insecticides and insect repellents have been developed. Mosquitoes can be targeted with insecticides when they are in a larval state or once they have developed into adults. Accordingly, insecticides which are used to kill larvae are termed larvicides whereas insecticides that are used to specifically target adult insects are called adulticides. Most of the insecticides commonly used to prevent the spread of disease are targeted against the adult mosquito and in particular against the female adult mosquito.

The organochlorine DDT was the most widespread compound used worldwide as an adulticide until it was withdrawn from use in most areas. After that, organophosphates such as malathion, carbamates, e.g., propoxur were widely used in vector control programmes in most parts of the world and were steadily replaced by pyrethroids, which became the mostly used adulticide.

Organophosphates, such as pirimiphos-methyl are now being used again due to the development of pyrethroid resistance in many important vector species.

One of the most important problems associated with pyrethroids, like their predecessors, is that resistance has already developed in many insect species in several parts of the world. Pyrethroid resistance, caused either by specific detoxification enzymes or an altered target site mechanism (kdr-type mutations in the sodium channels), has been reported in most continents in the majority of medically important mosquitoes species, such as Anopheles gambiae in Africa and Aedes aegypti in Asia. If resistance continues to develop and spread at the current rate, it may render such insecticides ineffective in their current form in the not too distant future. Such a scenario would have potentially devastating consequences in public health terms, since there are as yet no obvious alternatives to many of the uses of pyrethroids.

Therefore, there is an ongoing search for compounds for control of mosquitoes, especially for mosquitoes having developed resistance, such as against pyrethroids.

As well as the biological efficacy of the compounds of the present invention against mosquitoes and resistant strains of such mosquitoes, other considerations for selecting a suitable compound could include its safety (such as its toxicity, persistence) to the environment, including to the users of a vector control solution; its suitability for making a vector control solution product (whether indoor residual spray formulation, mosquito net, or another type), its suitability for adherence and availability on a surface over a period of time (in the event the solution is an indoor residual spray), and also its suitability for incorporation into a polymer product (such as a net) so that the compound would be readily available to control mosquitoes on the surface of the net over a period of time and the nets can withstand multiple washings.

Certain compounds of any one of the formulae (I), (I-1), (I-2) and (I-A1) have been found to be useful in control of mosquitoes. Such compounds are herein referred to as a COMPOUND M—as defined in the first aspect of the present invention.

A particularly preferred COMPOUND M is represented formula (I-1), wherein wherein R¹, R², R³, R⁴ and R⁵ are as defined in formula (I).

In an embodiment, compound M is a of formula (I-1) wherein R¹ is phenyl, phenyl substituted by 1 to 3 substituents independently selected from halogen, cyano, C₁-C₄-alkyl, C₁-C₄-haloalkyl, C₁-C₄-haloalkoxy, and C₁-C₄-alkoxy; R² is C₁-C₆-alkyl, cyclopropyl, phenyl, benzyl, phenylethyl, cyclopropylmethyl, or mono-substituted halophenyl; R³ is t-Butyl, C(CH₃)₂(C₂H₅), or C(CH₃)₂(CF₃); R⁴ is methyl or ethyl; and R⁵ is H.

In a preferred embodiment, compound M is a of formula (I-1), wherein R¹ is phenyl or phenyl substituted by one to three substituents independently selected halogen atoms; R² is C₂-C₅-alkyl or cyclopropyl; R³ is bridged C₃-C₅-alkyl, or C₃-C₅-haloalkyl, such as t-Butyl, C(CH₃)₂(C₂H₅), or C(CH₃)₂(CF₃); R⁴ is methyl; and R⁵ is H.

Especially preferred compounds M are compounds 3.1, 3.2, 3.4, 3.29, 3.30, 3.32, 3.57, 3.58, 3.60, 4.1, 4.2, 4.4, 4.29, 4.30, 4.32, 4.57, 4.58, 4.60, 5.1, 5.2, 5.4, 5.29, 5.30, 5.32, 5.57, 5.58, and 5.60 (from tables 3 to 5 above).

Mosquito vector control is any method to limit or eradicate mosquito species which transmit disease pathogens. The most frequent types of mosquito vector control employ a variety of strategies.

Mosquito vector control focuses on utilizing preventative methods to control or eliminate mosquito populations. Common preventative measures are
habitat control—removing or reducing areas where mosquitoes can easily breed can help limit population growth. For example, stagnant water removal, destruction of old tires and cans which serve as mosquito breeding environments and good management of stored water can reduce areas of excessive mosquito incidence.
reducing contact—limiting exposure to mosquitoes can reduce infection risks significantly. For example, bed nets, window screens on homes, or protective clothing can help reduce the likelihood contact with mosquitoes. To be effective this requires education and promotion of methods among the population to raise the awareness of mosquito threats.
chemical control—insecticides, larvicides, and repellents can be used to control mosquitoes. For example, larvicides can be used in mosquito breeding zones; insecticides can be applied to house walls or bed nets, and use of personal repellents can reduce incidence of mosquito bites and thus infection. The use of pesticides for mosquito vector control is promoted by the World Health Organization (WHO) and has proven to be highly effective.
biological control—the use of natural mosquito vector predators, such as bacterial toxins or botanical compounds, can help control mosquito populations. Using fish that eat mosquito larvae, has been demonstrated to have some success.
population control through the release of sterilized, or genetically modified, male mosquitoes has also been shown to control mosquito vector populations and reduce infection risks.

A number of considerations is taken into account when determining which COMPOUND M would be suitable for use in a particular mosquito vector control strategy, such as favourable safety profile, biological performance and affordability.

In one embodiment, a COMPOUND M in accordance with the methods and other aspects of the present invention are useful in controlling mosquitoes, in particular mosquitoes selected from the genus Anopheles, Culex and Aedes. Examples include Aedes aegypti, Aedes albopictus, Aedes japonicas, Aedes vexans, Coquillettidia perturbans, Culex molestus, Culex pallens, Culex pipiens, Culex quinquefasciatus, Culex restuans, Culex tarsalis, Anopheles albimanus, Anopheles albitarsis, Anopheles annularis, Anopheles aquasalis, Anopheles arabiensis, Anopheles aconitus, Anopheles atroparvus, Anopheles balabacensis, Anopheles culicifacies, Anopheles coluzzii, Anopheles darlingi, Anopheles dirus, Anopheles farauti, Anopheles flavirostris, Anopheles fluviatilis, Anopheles freeborni, Anopheles funestus, Anopheles gambiae s.l., Anopheles koliensis, Anopheles labranchiae, Anopheles lesteri, Anopheles leucosphyrus, Anopheles maculatus, Anopheles marajoara, Anopheles melas, Anopheles merus, Anopheles messeae, Anopheles minimus, Anopheles moucheti, Anopheles nili, Anopheles nuneztovari, Anopheles plumbeus, Anopheles pseudopunctipennis, Anopheles punctipennis, Anopheles punctulatus, Anopheles quadrimaculatus, Anopheles sacharovi, Anopheles sergentii, Anopheles sinensis, Anopheles stephensi, Anopheles subpictus, Anopheles sundaicus, Anopheles superpictus, and Mansonia titillans, Ochlerotatus stimulans, Ochlerotatus japonicas (each of which is an example of a mosquito capable of carrying or vectoring a pathogenic disease).

By control is meant that a COMPOUND M useful in the methods and other aspects of the invention is employed in a manner that kills or repels the mosquito such that biting does not occur or in a manner that decreases mosquito populations such that biting does not occur as frequently.

In an especially preferred embodiment, a COMPOUND M, selected from the group consisting of compound numbers 3.1, 3.2, 3.4, 3.29, 3.30, 3.32, 3.57, 3.58, 3.60, 4.1, 4.2, 4.4, 4.29, 4.30, 4.32, 4.57, 4.58, 4.60, 5.1, 5.2, 5.4, 5.29, 5.30, 5.32, 5.57, 5.58, and 5.60 (from tables 3 to 5 above), is useful in controlling a mosquito selected from the genus Anopheles, Culex and Aedes, in particular Aedes aegypti, Aedes albopictus, Aedes japonicas, Aedes vexans, Culex molestus, Culex pallens, Culex pipiens, Culex quinquefasciatus, Culex restuans, Culex tarsalis, Anopheles albimanus, Anopheles arabiensis, Anopheles coluzzii, Anopheles darlingi, Anopheles dirus, Anopheles funestus, Anopheles gambiae s.l., Anopheles melas, Anopheles minimus, Anopheles sinensis, Anopheles stephensi, Mansonia titillans.

In an embodiment, the COMPOUND M is useful in the methods and other aspects of the invention to control adult mosquitoes.

In another embodiment the following COMPOUND M are especially useful in controlling one or more of the mosquitoes listed in table M below:

| Compound nos. | Mosquito species |
|---|---|
| 3.1, 3.2, 3.4, 3.29, 3.30, 3.32, 3.57, 3.58 or 3.60 | Aedes aegypti |
| 3.1, 3.2, 3.4, 3.29, 3.30, 3.32, 3.57, 3.58 or 3.61 | Anopheles funestus |
| 3.1, 3.2, 3.4, 3.29, 3.30, 3.32, 3.57, 3.58 or 3.62 | Anopheles gambiae |
| | s.I. |
| 3.1, 3.2, 3.4, 3.29, 3.30, 3.32, 3.57, 3.58 or 3.63 | Anopheles stephensi |
| 3.1, 3.2, 3.4, 3.29, 3.30, 3.32, 3.57, 3.58 or 3.64 | Anopheles arabiensis |
| 3.1, 3.2, 3.4, 3.29, 3.30, 3.32, 3.57, 3.58 or 3.65 | Aedes albopictus |
| 3.1, 3.2, 3.4, 3.29, 3.30, 3.32, 3.57, 3.58 or 3.66 | Anopheles coluzzii |
| 4.1, 4.2, 4.4, 4.29, 4.30, 4.32, 4.57, 4.58 or 4.60 | Aedes aegypti |
| 4.1, 4.2, 4.4, 4.29, 4.30, 4.32, 4.57, 4.58 or 4.60 | Anopheles funestus |
| 4.1, 4.2, 4.4, 4.29, 4.30, 4.32, 4.57, 4.58 or 4.60 | Anopheles gambiae |
| | s.I. |
| 4.1, 4.2, 4.4, 4.29, 4.30, 4.32, 4.57, 4.58 or 4.60 | Anopheles stephensi |
| 4.1, 4.2, 4.4, 4.29, 4.30, 4.32, 4.57, 4.58 or 4.60 | Anopheles arabiensis |
| 4.1, 4.2, 4.4, 4.29, 4.30, 4.32, 4.57, 4.58 or 4.60 | Aedes albopictus |
| 4.1, 4.2, 4.4, 4.29, 4.30, 4.32, 4.57, 4.58 or 4.60 | Anopheles coluzzii |
| 5.1, 5.2, 5.4, 5.29, 5.30, 5.32, 5.57, 5.58 or 5.60 | Aedes aegypti |
| 5.1, 5.2, 5.4, 5.29, 5.30, 5.32, 5.57, 5.58 or 5.60 | Anopheles funestus |
| 5.1, 5.2, 5.4, 5.29, 5.30, 5.32, 5.57, 5.58 or 5.60 | Anopheles gambiae |
| | s.I. |
| 5.1, 5.2, 5.4, 5.29, 5.30, 5.32, 5.57, 5.58 or 5.60 | Anopheles stephensi |
| 5.1, 5.2, 5.4, 5.29, 5.30, 5.32, 5.57, 5.58 or 5.60 | Anopheles arabiensis |
| 5.1, 5.2, 5.4, 5.29, 5.30, 5.32, 5.57, 5.58 or 5.60 | Aedes albopictus |
| 5.1, 5.2, 5.4, 5.29, 5.30, 5.32, 5.57, 5.58 or 5.60 | Anopheles coluzzii |

Insecticide resistant mosquito species have also been detected and accordingly in an embodiment, the COMPOUND M useful in the methods and other aspects of the invention is suitable for controlling insecticide-resistant mosquitoes, such as pyrethroid and/or carbamate-resistant mosquitoes.

Pyrethroids are the only insecticides that have obtained WHO recommendation against malaria vectors on both Indoor Residuals Sprays (IRS) and Long Lasting Insecticidal Mosquito Nets (LLINs), in the form of alpha-cypermethrin, bifenthrin, cyfluthrin, permethrin, deltamethrin, lambda-cyhalothrin and etofenprox. It has been the chemical class of choice in agriculture and public health applications over the last several decades because of its relatively low toxicity to humans, rapid knock-down effect, relative longevity (duration of 3-6 months when used as IRS), and low cost. However, massive use of pyrethroids in agricultural applications and for vector control led to the development of resistance in major malaria and dengue vectors. Strong resistance has, e.g., been reported for the pyrethroid deltamethrin (and permethrin) for the Anopheles gambiae Tiassalé (from southern Cote d'lvoire) strain (Constant V. A. Edi et al., Emerging Infectious Diseases; Vol. 18, No. 9, September 2012). Pyrethroid resistance was also reported for permethrin, deltamethrin and lam bda-cyhalothrin for the Aedes aegypti Cayman Island strain (Angela F. Harris et al., Am. J. Trop. Med. Hyg., 83(2), 2010) and alpha-cypermethrin, permethrin and lambda-cyhalothrin for certain Anopheles strains (Win Van Bortel, Malaria Journal, 2008, 7:102).

In another embodiment of the invention, the COMPOUND M can be suitable for use against insecticide-resistant mosquitoes that are selected from Anopheles gambiae RSPH, Anopheles gambiae Tiassalé, Anopheles gambiae Akron, Anopheles gambiae Kisumi Rdl, Anopheles arabiensis NDjamina, Anopheles coluzzii VK7, Anopheles funestus FUMOZ, Aedes aegypti Grand Cayman and Culex quinquefasciatus strain POO.
Anopheles gambiae, strain RSPH is a multi-resistant mosquito (target-site and metabolic-resistance) that is described in the reagent catalogue of the Malaria Research and Reference Reagent Resource Center (www.MR4.org; MR4-number: MRA-334).
Anopheles gambiae, strain Tiassalé is a multi-resistant mosquito (target and metabolic-resistant strain) which shows cross-resistance between carbamates, organophosphates and pyrethroids and is described in Constant V.A. Edi et al., Emerging Infectious Diseases; Vol. 18, No. 9, September 2012 and Ludovic P Ahoua Alou et al., Malaria Journal 9: 167, 2010).
Anopheles gambiae, strain Akron is a multi-resistant mosquito (target and metabolic-resistant strain) and is described in Djouaka F Rousseau et al., BMC Genomics, 9:538; 2008.
Anopheles coluzzii, strain VK7 is a mosquito population with target site and metabolic resistance mechanisms, and is described in Toé et al, BMC Genomics 2015, 16:146.
Anopheles funestus, strain FUMOZ is a metabolic-resistant strain and is described in Hunt et al., Med Vet Entomol. 2005 September; 19(3):271-5). In this article it has been reported that Anopheles funestus—as one of the major malaria vector mosquitoes in Africa—showed resistance to pyrethroids and carbamate insecticides in South Africa.
Anopheles gambiae, strain Kisumi Rdl, a dieldrin resistant strain from Kenya.
Anopheles arabiensis, strain NDjamina, a pyrethroid resistant from Chad.
Aedes aegypti, strain Grand Cayman is a target-resistant mosquito and is described in Angela F. Harris, Am. J. Tro. Med. Hyg. 83(2), 2010.
Culex quinquefasciatus (metabolic-resistant to DDT strain P00); received from Texchem, Penang, Malaysia.

Vector control management methods or control solutions are means to control a vector, such as a mosquito. Examples of such methods include use of compositions, products, and treated articles of the present invention, such as a substrate or non-living material incorporating (e.g., coated or impregnated with) a COMPOUND M, spray products (e.g., indoor sprays) comprising a COMPOUND M, paint compositions comprising a COMPOUND M, and products or treated articles comprising a COMPOUND M.

Examples of integrated mosquito vector management methods or control solutions of the invention, such as methods for controlling mosquito bites or decreasing relevant mosquito populations, include the use of such compositions, products, treated articles and substrates of the invention at a locus of potential or known interaction between the mosquito vector and an animal, including a human, that is susceptible to a pathogenic disease infection transmitted by such vector. Suitable integrated solutions within the scope of the present invention also include identifying mosquito breeding sites and positioning compositions, products, treated articles and substrates of the invention at such sites.

Examples of a substrate or non-living material of the invention are self-supporting film/sheet (e.g., screens), threads, fibres, yarns, pellets, weaves (or textiles (e.g., for clothing)), nets, tents, and curtains incorporating (e.g., coated or impregnated with) a COMPOUND M, which can be used to protect against mosquito bites. In particular, it is well known that humans can be protected in their sleep from mosquito stings by insecticidally coated sleeping nets. Coated or impregnated weaves of the invention can also be used as curtains in front of windows, doors open eaves, or ventilation openings, in order to control mosquito entering dwellings.

The use of a compound in a substrate of the present invention (e.g., nets and weaves) achieves at least one of the following objects:
good insecticidal effect
fast-acting insecticidal efficacy
long-lasting insecticidal efficacy
uniform release of active ingredient
long durability (including resisting multiple washings over an extended period)
simple production
safe to the user

The nets and weaves (or textiles) of the invention that incorporate (e.g., are coated or impregnated with) a COMPOUND M are made up of a variety of natural and synthetic fibres, also as textile blends in woven or non-woven form, as knit goods or fibres. Natural fibres are, for example, raffia, jute, flax, sisal, hessian, wool, silk or hemp. Synthetic fibres may be made of polyamides, polyesters, polyacrylonitriles, polyolefines, for example polypropylene or polyethylene, Teflon, and mixtures of fibres, for example mixtures of synthetic and natural fibres. Polyamides, polyolefins and polyesters are preferred as fibre material. Polyester, such a polyethylene terephthalate, polyethylene and polypropylene are especially preferred. Most preferred are nettings made from polyethylene and/or polypropylene.

The art discloses methods suitable for incorporating (by way of coating) a compound onto nets and weaves (see for example, WO2003/034823, WO 2008/122287, WO 01/37662, US2009036547, WO 2007/036710), from dipping or submerging them into a formulation of the insecticide or by spraying the formulation onto their surfaces. After treating the nets and weaves of the invention, they may be dried simply at ambient temperatures (see also below for more background). Such methods are also suitable for incorporating (by way of coating) a COMPOUND M.

Also disclosed in the art are methods suitable for incorporating (by way of impregnating) a pesticide compound within the net or weave by making polymer material in the presence of the compound, which is then extruded into fibres, threads or yarns, for making the nets and weaves (see for example, WO08004711, WO2009/121580, WO2011/128380, WO2011/141260, WO2010/118743). Such nets and weaves having available at the surface of the net and weave an effective amount of the compound so as to control mosquito bites. Generally the compound is mixed with the molten polymer. Such methods are also suitable for incorporating (by way of impregnating) a COMPOUND M.

The term “incorporating” or “incorporated” in context of the compound of the invention, additives and other insecticides is meant that the substrate or non-living material comprises or contains the respectively defined compound, additive and/or insecticide, such as by coating or impregnation.

Preferably the substrate of the present invention is a net, which net is preferably a long lasting net, incorporated with a COMPOUND M by way of coating the net with a composition comprising a COMPOUND M, or by way of making a polymeric material in the presence of such a COMPOUND M and then processing the resultant polymeric material into an inventive net.

In accordance with the invention, when a COMPOUND M is used within the polymer, then during use of the resulting net or weave made from the polymer, the COMPOUND M is released to the surface of the net to control against mosquito bites—such control is sustained at adequate level and for adequate amount of time.

Examples of suitable polymers are polyamides, polyesters, polyacrylonitriles, polyolefines, such as polyethylene compositions that can be made from different polyethylene polymers; these may be LDPE, LLDPE, MDPE and HDPE. LLDPE (Linear low-density polyethylene) is a substantially linear polymer (polyethylene), with significant numbers of short branches, commonly made by copolymerization of ethylene with longer-chain olefins. MDPE is medium-density polyethylene is a substantially linear polymer of polyethylene with shorter chain length than HDPE. HDPE (High-Density PolyEthylene) or PolyEthylene High-Density (PEHD) is a polyethylene thermoplast. HDPE has little branching, giving it stronger intermolecular forces and tensile strength than lower-density polyethylene. It is also harder and more opaque and can withstand somewhat higher temperatures (120 degrees Centigrade/248 degrees Fahrenheit for short periods, 110 degrees Centigrade/230 degrees Fahrenheit continuously). HDPE yarns are stronger than LDPE mixed polyethylene yarns. LLDPE differs structurally from conventional low-density polyethylene (LDPE) because of the absence of long chain branching. These polyethylene compositions (HDPE, LDPE, LLDPE and mixture thereof) are generally used for preparing yarns and polyethylene based textile products. Methods for incorporating an insecticide compound into the polymer without weakening its resulting properties are known in the art, such as using mixtures of HDPE and LDPE. Such methods can also be used to incorporate a COMPOUND M into a polymer.

Examples of spray products of the present invention are indoor residual sprays or space sprays comprising a COMPOUND M. Indoor Residual Spraying (IRS) is the technique of applying a residual deposit of an insecticide onto indoor surfaces where vectors rest, such as on walls and ceilings. The primary goal of indoor residual spraying is to reduce the lifespan of the mosquito vectors and thereby reduce or interrupt disease transmission. The secondary impact is to reduce the density of mosquitoes within the treatment area. IRS is a recognised, proven and cost-effective intervention method for the control of malaria and it is also used in the management of Leishmaniasis disease. Many malaria mosquito vectors are endophilic, resting inside houses after taking a blood meal. These mosquitoes are particularly susceptible to control through indoor residual spraying (IRS) comprising a COMPOUND M. As its name implies, IRS involves coating the walls and other surfaces of a house with a residual insecticide. For several months, the COMPOUND M will kill mosquitoes that come in contact with these surfaces. IRS does not directly prevent people from being bitten by mosquitoes. Rather, it usually kills mosquitoes after they have fed, if they come to rest on the sprayed surface. IRS thus prevents transmission of infection to other persons. To be effective, IRS must be applied to a very high proportion of households in an area (usually greater than 70 percent). Although the community plays a passive role in IRS programs, cooperation with an IRS effort is a key to its success. Community participation for IRS often consists of cooperating with the spray teams by removing food and covering surfaces prior to spraying and refraining from covering the treated surfaces with new paint or plaster. However, community or individual householder opposition to IRS due to the smell, mess, possible chemical exposure, or sheer bother has become a serious problem in some areas. Therefore, sprays in accordance with the invention having good residual efficacy and acceptable odour are particularly suited as a component of integrated mosquito vector management or control solutions.

In contrast to IRS, which requires that the active COMPOUND M is bound to surfaces of dwellings, such as walls, ceiling, space spray products of the invention rely on the production of a large number of small insecticidal droplets intended to be distributed through a volume of air over a given period of time. When these droplets impact on a target mosquito, they deliver a lethal dose of the COMPOUND M. The traditional methods for generating a space-spray include thermal fogging (whereby a dense cloud of insecticide droplets is produced giving the appearance of a thick fog) and Ultra Low Volume (ULV), whereby droplets are produced by a cold, mechanical aerosol-generating machine.

Since large areas can be treated at any one time this method is a very effective way to rapidly reduce the population of flying mosquitoes in a specific area. Since there is very limited residual activity from the application it must be repeated at intervals of 5-7 days in order to be fully effective. This method can be particularly effective in epidemic situations where rapid reduction in mosquito numbers is required. As such, it can be used in urban dengue control campaigns.

Effective space-spraying is generally dependent upon the following specific principles:
Target insects are usually flying through the spray cloud (or are sometimes impacted whilst resting on exposed surfaces). The efficiency of contact between the spray droplets and target insects is therefore crucial. This is achieved by ensuring that spray droplets remain airborne for the optimum period of time and that they contain the right dose of insecticide. These two issues are largely addressed through optimizing the droplet size.
If droplets are too big they drop to the ground too quickly and don't penetrate vegetation or other obstacles encountered during application (limiting the effective area of application). If one of these big droplets impacts an individual insect then it is also ‘overkill’ since a high dose will be delivered per individual insect.
If droplets are too small then they may either not deposit on a target insect (no impaction) due to aerodynamics or they can be carried upwards into the atmosphere by convection currents.
The optimum size of droplets for space-spray application are droplets with a Volume Median Diameter (VMD) of 10-25 microns.

The compositions of the present invention may be made available in a spray product as an aerosol-based application, including aerosolized foam applications. Pressurised cans are the typical vehicle for the formation of aerosols. An aerosol propellant that is compatible with the insecticide compound is used. Preferably, a liquefied-gas type propellant is used. Suitable propellants include compressed air, carbon dioxide, butane and nitrogen. The concentration of the propellant in the COMPOUND M composition is from about 5 percent to about 40 percent by weight of the COMPOUND M composition, preferably from about 15 percent to about 30 percent by weight of the COMPOUND M composition.

In one embodiment, the COMPOUND M formulation of the invention can also include one or more foaming agents. Foaming agents that can be used include sodium laureth sulphate, cocamide DEA, and cocamidopropyl betaine. Preferably, the sodium laureth sulphate, cocamide DEA and cocamidopropyl are used in combination. The concentration of the foaming agent(s) in the COMPOUND M composition is from about 10 percent to about 25 percent by weight, more preferably 15 percent to 20 percent by weight of the composition.

When the COMPOUND M formulation is used in an aerosol application not containing foaming agents), the composition of the present invention can be used without the need for mixing directly prior to use. However, aerosol formulations containing the foaming agents do require mixing (i.e. shaking) immediately prior to use. In addition, if the formulations containing foaming agents are used for an extended time, they may require additional mixing at periodic intervals during use.

A dwelling area may also be treated with the COMPOUND M composition of the present invention by using a burning formulation, such as a candle, a smoke coil or a piece of incense containing the composition. For example, composition may be comprised in household products such as “heated” air fresheners in which insecticidal compositions are released upon heating, for example, electrically, or by burning.

The compositions of the present invention containing a COMPOUND M may be made available in a spray product as an aerosol, a mosquito coil, and/or a vaporiser or fogger.

The concentration of COMPOUND M in the polymeric material, fibre, yarn, weave, net, or substrate, each of the invention, can be varied within a relatively wide concentration range from, for example 0.05 to 15 percent by weight, preferably 0.2 to 10 percent by weight, more preferably 0.4 to 8 percent by weight, especially 0.5 to 5, such as 1 to 3, percent by weight.

The percentages mentioned above are based on dry weight of the net or substrate or non-living material.

Similarly, the concentration of the compound of the invention in the composition (whether for treating surfaces or for coating a fibre, yarn, net, weave) can be varied within a relatively wide concentration range from, for example 0.1 to 70 percent by weight, such as 0.5 to 50 percent by weight, preferably 1 to 40 percent by weight, more preferably 5 to 30 percent by weight, especially 10 to 20 percent by weight.

The concentration shall be chosen according to the field of application such that the requirements concerning insecticidal efficacy, durability and toxicity are met. Adapting the properties of the material can also be accomplished and so custom-tailored textile fabrics are obtainable in this way.

The COMPOUND M when used in the IRS methods of the invention is present on a surface of a dwelling at a coverage of from 0.01 to 2 grams of Al per m2, preferably from 0.05 to 1 grams of Al per m2, especially from 0.1 to 0.7 grams of Al per m2.

Accordingly an effective amount of a COMPOUND M can depend on how it is being used, the mosquito against which control is most desired and the environment it is being used in. Therefore, an effective amount of a COMPOUND M is sufficient that control of a mosquito is achieved; in case of:
use as a IRS formulation, the effective amount is such that coverage of the Al on the surface is from 0.01 to 2 grams of Al per m2, preferably from 0.05 to 1 grams of Al per m2, especially from 0.1 to 0.7 grams of Al per m2;
use incorporated within a net or substrate, the effective amount is 0.05 to 15 percent by weight, preferably 0.2 to 10 percent by weight, more preferably 0.4 to 8 percent by weight, especially 0.5 to 5, such as 1 to 3, percent by weight.

Generally the COMPOUND M when used in certain products of the invention is continuously distributed in a thread, yarn, net or weave, but can also be partially or discontinuously distributed in a thread, yarn, net or weave. For example, a net may contain certain parts which are coated or which is made-up of impregnated fibre, and certain other parts which are not; alternatively some of the fibres making up the net is impregnated, or is coated, with the compound of the invention, and some of the other fibres not or these other fibres are impregnated, or are coated, with another insecticide compound (see below).

Nets of the invention impregnated, or coated, with a COMPOUND M can satisfy the criteria of the WHOPES directive (see “Guidelines for laboratory and field testing of long-lasting insecticidal mosquito nets”, 2005, http://www.who.int/whopes/guidelines/en/) for insecticide-containing long-lasting mosquito nets up to 20 washes only, which means that such nets should not lose their biological activity after just 20 wash cycles or so.

In an embodiment, a net of the invention impregnated, or coated, with a COMPOUND M can have biological activity in accordance with WHOPES guidelines of a knockdown after 60 minutes of between 95 percent and 100 percent or a mortality after 24 hours of between 80 percent and 100 percent after at least 20, such as 25, preferably at least 30 and even more preferably at least 35 washes.

The “WHOPES directive” is to be understood as meaning the directive “Guidelines for laboratory and field testing of long-lasting insecticidal mosquito nets”, 2005). This directive is retrievable at the following interact address: http://www.who.int/whopes/guidelines/en/.

When a net is “impregnated with” a COMPOUND M to prepare a net of the present invention, the fibres making up the net are made by melting a polymer, a COMPOUND M and optionally other compounds, such as other insecticides, additives, stabilisers. When a net is impregnated with a COMPOUND M, then the net of the invention contains synthetic fibres; in contrast, a net of the invention coated with a COMPOUND M contains synthetic fibres and/or natural fibres.

The polymeric materials useful in the compositions of the invention incorporating a COMPOUND M can be produced by mixing such a COMPOUND M with the polymer in the liquid phase, and optionally other additives (such as binders and/or synergists), and other insecticidal compounds.

Methods of making suitable polymeric materials and then processing it are described in the art—see for example, WO09121580, WO2011/141260.

For example, nets based on an insecticide-containing polymeric material are produced by the following steps:
a) melting the polymer to be used and one or more insecticidally active ingredients together or separately at temperatures between 120 and 250 degrees centigrade,
b) forming the melt of step a) into spun threads and cooling,
c) optionally leading the spun threads formed in step b) through a drawing system and drawing and then optionally setting out the threads,
d) knitting the spun threads to form a net,
e) subjecting the net to a heat-setting operation wherein the temperature for the heat-setting operation is chosen to be 20 degrees centigrade below the melting temperature of the polymer to be used.

The heat setting in step e) of the production of the nets is preceded by a washing step. Water and a detergent is preferably used for this. The heat setting is preferably carried out in a dry atmosphere.

Although the manufacture of the nets incorporated with a compound can occur in a single location, it is also envisaged that the different steps can take place in different locations. So a composition comprising a compound M may be made which can then be processed into a polymer. Accordingly, the present invention also provides a composition comprising a compound M in a concentrated form, which composition may also contain additives (such as binders and/or synergists), and other insecticidal compound(s) (which composition had been prepared explicitly for making a polymer material impregnated with the compound M (such a composition is often referred to as a “masterbatch”)). The amount of the compound defined in the first aspect in the masterbatch would depend on the circumstances, but in general can be 10 to 95 percent by weight, such as 20 to 90 percent by weight, preferably 30 to 85 percent by weight, more preferably 35 to 80 percent by weight, especially 40 to 75 percent by weight.

Also made available in the present invention are compositions or formulations for coating walls, floors and ceilings inside of buildings and for coating a substrate or non-living material, which comprise a COMPOUND M. The inventive compositions can be prepared using known techniques for the purpose in mind, which could contain a binder to facilitate the binding of the compound to the surface or other substrate. Agents useful for binding are known in the art and tend to be polymeric in form. The type of binder suitable for composition to be applied to a wall surface having particular porosities, binding characteristics would be different to a fibre, yarn, weave or net—a skilled person, based on known teachings, would select a suitable binder.

Typical binders are poly vinyl alcohol, modified starch, poly vinyl acrylate, polyacrylic, polyvinyl acetate co polymer, polyurethane, and modified vegetable oils. Suitable binders can include latex dispersions derived from a wide variety of polymers and co-polymers and combinations thereof. Suitable latexes for use as binders in the inventive compositions comprise polymers and copolymers of styrene, alkyl styrenes, isoprene, butadiene, acrylonitrile lower alkyl acrylates, vinyl chloride, vinylidene chloride, vinyl esters of lower carboxylic acids and alpha, beta-ethylenically unsaturated carboxylic acids, including polymers containing three or more different monomer species copolymerized therein, as well as post-dispersed suspensions of silicones or polyurethanes. Also suitable may be a polytetrafluoroethylene (PTFE) polymer for binding the active ingredient to other surfaces.

The formulation according to the present invention comprises at least one COMPOUND M (or a pesticide (A), and a carrier, such as water (C), and optionally a polymeric binder (B) and further components (D).

The polymeric binder binds the COMPOUND M to the surface of the non-living material and ensures a long-term effect. Using the binder reduces the elimination of the COMPOUND M pesticide out of the non-living material due to environmental effects such as rain or due to human impact on the non-living material such as washing and/or cleaning it. The further components can be an additional insecticide compound, a synergist, a UV stabiliser.

The inventive compositions can be in a number of different forms or formulation types, such as suspensions, capsules suspensions, and a person skilled in the art can prepare the relevant composition based on the properties of the particular COMPOUND M, its uses and also application type.

For example, the COMPOUND M used in the methods and other aspects of the present invention may be encapsulated in the formulation. A encapsulated compound can provide improved wash-fastness and also longer period of activity. The formulation can be organic based or aqueous based, preferably aqueous based.

Microencapsulated COMPOUND M compounds suitable for use in the compositions and methods according to the invention are prepared with any suitable technique known in the art. For example, various processes for microencapsulating material have been previously developed. These processes can be divided into three categories-physical methods, phase separation and interfacial reaction. In the physical methods category, microcapsule wall material and core particles are physically brought together and the wall material flows around the core particle to form the microcapsule. In the phase separation category, microcapsules are formed by emulsifying or dispersing the core material in an immiscible continuous phase in which the wall material is dissolved and caused to physically separate from the continuous phase, such as by coacervation, and deposit around the core particles. In the interfacial reaction category, microcapsules are formed by emulsifying or dispersing the core material in an immiscible continuous phase and then an interfacial polymerization reaction is caused to take place at the surface of the core particles. The concentration of the COMPOUND M present in the microcapsules can vary from 0.1 to 60% by weight of the microcapsule.

The formulation according to the invention may be formed by mixing all ingredients together with water optionally using suitable mixing and/or dispersing aggregates. In general, the formulation is formed at a temperature of from 10 to 70 degrees centigrade, preferably 15 to 50 degrees centigrade, more preferably 20 to 40 degrees centigrade

It is possible to use a pesticide (A), solid polymer (B) and optionally additional additives (D) and to disperse them in the aqueous component (C)

If a binder is present in a composition of the present invention, it is preferred to use dispersions of the polymeric binder (B) in water as well as aqueous formulations of the pesticide (A) in water which have been separately prepared before. Such separate formulations may contain additional additives for stabilizing (A) and/or (B) in the respective formulations and are commercially available. In a second process step, such raw formulations and optionally additional water (component (C)) are added.

Also combinations are possible, i.e. using a pre-formed dispersion of (A) and/or (B) and mixing it with solid (A) and/or (B).

A dispersion of the polymeric binder (B) may be a pre-manufactured dispersion already made by a chemicals manufacturer.

However, it is also within the scope of the present invention to use “hand-made” dispersions, i.e. dispersions made in small-scale by an end-user. Such dispersions may be made by providing a mixture of about 20 percent of the binder (B) in water, heating the mixture to temperature of 90 to 100 degrees centigrade and intensively stirring the mixture for several hours.

It is possible to manufacture the formulation as a final product so that it can be readily used by the end-user for the process according to the present invention.

However, it is of course also possible to manufacture a concentrate, which may be diluted by the end-user with additional water (C) to the desired concentration for use.

In an embodiment, a composition suitable for IRS application or a coating formulation containing a COMPOUND M contains the active ingredient and a carrier, such as water, and may also one or more co-formulants selected from a dispersant, a wetter, an anti-freeze, a thickener, a preservative, an emulsifier and a binder or sticker.

The COMPOUND M is generally milled to a desired particle size, such as the particle size distribution d(0.5) is generally from 3 to 20, preferably 5 to 15, especially 7 to 12, m.

Furthermore, it may be possible to ship the formulation to the end-user as a kit comprising at least
a first component comprising at least one COMPOUND M (A); and
a second component comprising at least one polymeric binder (B).
Further additives (D) may be a third separate component of the kit, or may be already mixed with components (A) and/or (B).

The end-user may prepare the formulation for use by just adding water (C) to the components of the kit and mixing.

The components of the kit may also be formulations in water. Of course it is possible to combine an aqueous formulation of one of the components with a dry formulation of the other component(s).

As an example, the kit can comprise
one formulation of a COMPOUND M (A) and optionally water (C); and
a second, separate formulation of at least one polymeric binder (B), water as component (C) and optionally components (D).

Accordingly, in a further aspect the present invention provides a kit for treating a fibre, yarn, net and weave by coating wash resistant insecticidal properties thereto comprising: a first sachet comprising a pre-measured amount of at least one COMPOUND M, and a second sachet comprising a pre-measured amount of at least one polymeric binder. The resulting treated fibre, yarn, net and weave has imparted thereto the insecticidal properties needed for vector control, such as to control vector-carrying mosquitoes.

The concentrations of the components (A), (B), (C) and optionally (D) will be selected by the skilled artisan depending of the technique to be used for coating/treating.

In general, the amount of pesticide (A) may be up to 50, preferably 5 to 50, such as 10 to 40, especially 15 to 30, percent by weight, based on weight of the composition.

The amount of polymeric binder (B) may be in the range of 0.01 to 30, preferably 0.5 to 15, more preferably 1 to 10, especially 1 to 5, percent by weight, based on weight of the composition.

If present, in general the amount of additional components (D) is from 0.1 to 20, preferably 0.5 to 15, percent by weight, based on weight of the composition. If present, suitable amounts of pigments and/or dyestuffs are in general 0.01 to 5, preferably 0.1 to 3, more preferably 0.2 to 2, percent by weight, based on weight of the composition.

A typical formulation ready for use comprises 0.1 to 40, preferably 1 to 30, percent of components (A), (B), and optionally (D), the residual amount being water (C).

A typical concentration of a concentrate to be diluted by the end-user may comprise 5 to 70, preferably 10 to 60, percent of components (A), (B), and optionally (D), the residual amount being water (C).

The formulation of the present invention may be applied to polymeric material before their formation into the required products, e.g., while still a yarn or in sheet form, or after formation of the relevant products.

For the case of nets and/or weaves, a process for coating nets and/or weaves at least comprising the following steps:

a) treating the nets and/or weaves with the aqueous formulation according to the invention by any of the procedural steps selected from the group of
(a1) passing the material through the formulation; or
(a2) contacting the material with a roller that is partly or fully dipped into the formulation and drawing the formulation to the side of the material in contact with the roller, or
(a3) submerging the material into the formulation; or
(a4) spraying the formulation onto the material; or
(a5) brushing the formulation onto or into the material; or
(a6) applying the formulation as a foam; or
(a7) coating the formulation onto material.

b) optionally removing surplus formulation by squeezing the material between rollers or by means of a doctor blade; and

c) drying the material.

In case the raw materials containing residues of preceding production processes, e.g., sizes, spin finishes, other auxiliaries and/or impurities, it may be beneficial to perform a washing step before the coating.

Specifically, the following details are important for the steps a), b), and c).

Step a1)

The formulation is applied by passing the material through the aqueous formulation. Said step is known by a person skilled in the art as padding. In a preferred embodiment the material is completely submerged in the aqueous formulation either in a trough containing the liquor or the material is passed through the formulation which is held between two horizontally oriented rollers. In accordance with the invention, the material may either be passed through the formulation or the formulation may be passed through the material. The amount of uptake of the formulation will be influenced by the stability of concentrated baths, the need for level distribution, the density of material and the wish to save energy costs for drying and curing steps. Usual liquor-uptakes may be 40 to 150 percent on the weight of material. A person skilled in the art is familiar with determining the optimum value. Step a1) is preferred for coating open-width material which is later tailored into nets.

For small-scale production or re-coating of non-treated nets, use of a simple hand-held roller may be sufficient.

Step a2)

It is further possible to apply the aqueous formulation on the material by a roller that is partly dipped into the dispersion thus applying the dispersion to the side of the material in contact with the roller (kiss-rolling). By this method it is possible to coat only one side of the material which is advantageous if, e.g., direct contact of the human skin with insecticide-treated material is to be avoided.

Coating of the material in step a1), a2) or a3) is typically carried out at temperatures from 10 to 70 degrees centigrade, preferably 15 to 50 degrees centigrade, more preferably 20 to 40 degrees centigrade

Step a4)

The spray may be applied in continuous processes or in batch-wise processes in suitable textile machines equipped with a spraying device, e.g., in open-pocket garment washer/extractors. Such equipment is especially suitable for impregnating ready-made nets.

Step a6)

A foam comprises less water than the dispersion mentioned above. The drying process may therefore be very short. The treatment may be performed by injecting gas or blends of gas (e.g., air) into it. The addition of surfactants, preferably with film-forming properties, may be required. Suitable surfactants and the required technical equipment are known to persons skilled in the art.

Step a7)

A coating process may preferably carried out in a doctor-blade process. The process conditions are known to a person skilled in the art.

Step b)

The surplus emulsion is usually removed by squeezing the material, preferably by passing the material through rollers as known in the art thus achieving a defined liquor uptake. The squeezed-off liquor may be re-used. Alternatively, the surplus aqueous emulsion or aqueous dispersion may be removed by centrifuging or vacuum suction.

Step c)

Drying may be performed at ambient temperatures. In particular, such a passive drying may be carried out in hot-dry climate. Of course, the drying process may be accelerated applying elevated temperatures. An active drying process would normally be performed during high scale processing. The drying is in general carried out temperatures below 200 degrees centigrade. Preferred temperatures are from 30 to 170 degrees centigrade, more preferably at room temperature. The temperature choice is determined by the thermal stability of the insecticide in the formulation and the thermal stability of the non-living material impregnated.

For the method according to the invention aqueous formulation comprising at least one pigment and/or at least one dyestuff may be used so that the material is not only coated with the COMPOUND M pesticide but in addition also coloured at the same time.

In a further aspect, the present invention provides a method for treating a fibre, yarn, net and weave by coating wash resistant insecticidal properties thereto comprising (i) preparing a treatment composition, which comprises at least one COMPOUND M, (ii) treating said fibre, yarn, net and weave and (iii) drying the resulting treated a fibre, yarn, net and weave.

The polymeric binder (B) can be dispersed in an aqueous formulation and comprises one or more fluorinated acrylic copolymers useful in the water and oil resistant formulations includes copolymer prepared by the polymerization of a perfluoroalkyl acrylate monomer and a comonomer, especially an acrylate monomer. The binder may also be fluorocarbon resins (as described in WO 2006/128870.

Only water is used as solvent for the formulation. However, trace amounts of organic solvents miscible with water may be present. Examples of solvents comprise water-miscible alcohols, e.g., monoalcohols such as methanol, ethanol or propanol, higher alcohols such as ethylene glycol or polyether polyols and ether alcohols such as butyl glycol or methoxypropanol. Preferably the content of an organic solvent is no more than 5 percent by weight (based on component (C), more preferably no more than 1 percent by weight (based on component (C), in particular no more than 0.1 percent by weight, based on component (C).

Depending on the intended use of the non-living material to be treated the formulation according to the present invention may further comprise one or more components or additives (D) selected from preservatives, detergents, fillers, impact modifiers, anti-fogging agents, blowing agents, clarifiers, nucleating agents, coupling agents, fixative agents, cross-linking agents, conductivity-enhancing agents (antistats), stabilizers such as antioxidants, carbon and oxygen radical scavengers and peroxide decomposing agents and the like, flame retardants, mould release agents, agents having UV protecting properties, spreading agents, anti-blocking agents, anti-migrating agents, foam-forming agents, anti-soiling agents, thickeners, further biocides, wetting agents, plasticizers and film-forming agents, adhesive or anti-adhesive agents, optical brightening (fluorescent whitening) agents, pigments and dyestuffs.

A typical amount of the polymeric binder (B) is from 0.01 to 10 percent by weight (dry weight) of the (dry) weight of the material. As a general guideline, the weight ratio between insecticide and binder (B) should approximately be constant with a value depending on the insecticidal and migratory ability of the insecticide, i.e. the higher the amount the insecticide the higher also the amount of binder (B). Preferred amounts of binder (B) are from 0.1 to 5 percent by weight, more preferably 0.2 to 3 percent by weight of the (dry) weight of the material.

The coated material can comprise at least one pigment and/or at least one dyestuff. The amount of the at least one pigment and/or dyestuff is in general from 0.05 to 10 percent by weight, preferably 0.1 to 5 percent by weight, more preferably 0.2 to 3.5 percent by weight of the (dry) weight of the material.

The method of coating or treating the non-living material is not limited to a specific technology. Coating may be performed by dipping or submerging the non-living substrate into the formulation or by spraying the formulation onto the surface of the non-living material. After treating the treated non-living substrate may be dried simply at ambient temperatures.

Accordingly, no sophisticated technology is necessary for the coating, and therefore the coating process may be carried out by the end-user itself in at low-scale.

For instance, a typical end-user may coat/treat a net itself, e.g., within its household, using the formulation according to the present invention. For this purpose, it is in particular advantageous to use a kit as herein defined.

In an embodiment, the present invention provides a polymer, a fibre, a thread, a yarn, a net or weave comprising one or more compounds M of the invention, where also incorporated can be one or more other customary materials used to make such a polymer, and the polymer, a fibre, a thread, a yarn, a net or weave optionally can further incorporate one or more other insecticides and/or synergists.

In an embodiment, the present invention provides a net or weave incorporated with one or more compounds M, which optionally further incorporates one or more other insecticides and/or synergists.

As described in the art, a compound useful in the methods and other aspects of the present invention can be used alone or in combination with another insecticide, synergist, insect repellent, chemosterilant, flame retardant, UV protector/absorber, and/or additives for controlling release characteristics.

When used in accordance with the invention, the COMPOUND M useful in the may be used alone to control a mosquito or used in combination with one or other known insecticides and/or one or more additives (such as synergists)—in polymers for making non-living substrates, such as nets and weaves, for formulations for treating non-living substrates, such as nets and weaves, in IRS products and space-spraying products.

In an embodiment, the present invention provides a composition (useful for coating a polymeric material or a product therefrom, or a useful as a spray product) comprising one or more compounds of the invention, which optionally further comprises one or more other insecticide and/or synergists and one or more other additives.

Examples of synergists are piperonylbutoxide (PBO), sebacic esters, fatty acids, fatty acid esters, vegetable oils, esters of vegetable oils, alcohol alkoxylates and antioxidants.

Suitable sebacic esters are for example dimethyl sebacate, diethyl sebacate, dibutyl sebacate, dibenzyl sebacate, bis(N-succinimidyl)sebacate, bis(2-ethylhexyl)sebacate, bis(1-octyloxy-2,2,6,6-tetramethyl-4-piperidyl)sebacate, bis(2,2,6,6-tetramethyl-4-piperidyl)sebacate and bis(1,2,2,6,6-pentamethyl-4-piperidinyl)sebacate (BLS292).

Suitable fatty acids are (preferably mono- or polyunsaturated) fatty acids having a chain length of 12 to 24 carbon atoms, for example palmitoleic acid, oleic acid, elaidic acid, vaccenic acid, icosenic acid, cetoleic acid, erucic acid, nervonic acid, linoleic acid, alpha-linolenic acid, gamma-linolenic acid, arachidonic acid, timnodonic acid, clupanodonic acid and cervonic acid. Particular preference is given to oleic acid, linoleic acid, alpha-linolenic acid and gamma-linolenic acid.

Suitable fatty acid esters are preferably methyl or ethyl esters of the above-recited fatty acids. Methyl esters are particularly preferred. Fatty acids and their esters can each also be present in mixtures.

Useful vegetable oils include all plant-derivable oils customarily usable in agrochemical compositions. As examples there may be mentioned sunflower oil, rapeseed oil, olive oil, castor oil, colza oil, maize kernel oil, cottonseed oil and soybean oil. Rapeseed oil is preferred.

Suitable esters of vegetable oils are methyl or ethyl esters of the above-recited oils. Methyl esters are preferred.

Antioxidants useful as additives include for example butylhydroxytoluene, butylhydroxyanisole and L-ascorbic acid.

Plant essential oils may also be used in an indoor residual spray compositions; examples are those selected from citronella, peppermint oil, d-limonene and Abies sibirica oil. These plant essential oil materials are known and used for other uses and can be prepared by a skilled artisan by employing known methods and also are available commercially.

In addition to at least one defined active ingredient of a COMPOUND M, the methods, compositions, polymer, product, substrate and/or integrated mosquito management solution according to the invention may contain one or more further insecticidally active ingredients. Particularly examples are one or more active ingredients from the class of organophosphates, pyrethroids, carbamates or neonicotinoids, and also DDT, indoxacarb, nicotine, bensultap, cartap, spinosad, camphechlor, chlordane, endosulfan, gamma-HCH, HCH, heptachlor, lindane, methoxychlor, acetoprole, ethiprole, fipronil, pyrafluprole, pyriprole, vaniliprole, avermectin, emamectin, emamectin-benzoate, ivermectin, milbemycin, diofenolan, epofenonane, fenoxycarb, hydroprene, kinoprene, methoprene, pyriproxifen, triprene, chromafenozide, halofenozide, methoxyfenozide, tebufenozide, bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron, buprofezin, cyromazine, diafenthiuron, azocyclotin, cyhexatin, fenbutatin-oxide, chlorfenapyr, binapacyrl, dinobuton, dinocap, DNOC, fenazaquin, fenpyroximate, pyrimidifen, pyridaben, tebufenpyrad, tolfenpyrad, hydramethylnon, dicofol, rotenone, acequinocyl, fluacrypyrim, Bacillus thuringiensis strains, spirodiclofen, spiromesifen, spirotetramat, 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS-Reg.-No.: 382608-10-8), flonicamid, amitraz, propargite, flubendiamide, chloranthraniliprol, thiosultap-sodium, azadirachtin, Bacillus spec., Beauveria spec., Metarrhizium spec., Paecilomyces spec., Thuringiensin, Verticillium spec., aluminium phosphide, methylbromide, sulfurylfluoride, cryolite, flonicamid, pymetrozine, clofentezine, etoxazole, hexythiazox, amidoflumet, benclothiaz, benzoximate, bifenazate, bromopropylate, buprofezin, chinomethionate, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonylbutoxide, kaliumoleat, pyridalyl, sulfluramid, tetradifon, tetrasul, triarathene and verbutin. In an embodiment, a preferred mixing partner is a pyrethroid, such as alpha-cypermethrin, bifenthrin, cyfluthrin, permethrin, deltamethrin, lambda-cyhalothrin and etofenprox.

In a further aspect, the present invention provides a method for protecting a mammal, including a human, against mosquitoes, the method comprising applying to the mosquito or to a locus of potential or known interaction between the mammal and the mosquito, a vector control management method or control solution comprising a mosquitocidally effective amount of a COMPOUND M.

Another aspect of the invention is a method for controlling the spread of a vector-borne disease, comprising: identifying a mosquito vector; and contacting the mosquito vector or its environment with a vector control management method or contro solution comprising a mosquitocidally effective amount of a COMPOUND M.

An aspect of the invention also includes a mosquitocidal method which comprises contacting a mosquito or its environment with a vector control management method or control solution comprising a mosquitocidally effective amount of a COMPOUND M.

The present invention also provides a method, comprising: (i) identifying a locus of potential or known interaction between a mosquito vector and a mammal, including a human, susceptible to pathogenic disease infection when contacted by such vector and (ii) positioning a vector control management method or control solution at the locus, wherein the solution includes a mosquitocidally effective amount of a COMPOUND M.

The present invention through control of mosquitoes would also be expected to control the many viruses carried by such vectors. As an example, control of the mosquitoes of the genus Aedes by use of one or more of a COMPOUND M, as part of a vector control management method or control solution, may control the Zika infections. Examples of mosquitoes reported to spread the Zika virus are the Aedes mosquitoes, such as Aedes aegypti and Aedes albopictus. Accordingly, in an aspect, the present invention provide a method of controlling Zika virus infection, wherein one or more of a COMPOUND M is present in a mosquitocidally effective amount in the vicinity of Aedes mosquitoes, such as Aedes aegypti and Aedes albopictus. In the vicinity of the mosquitoes is meant areas where mosquitoes are likely to be present, such as in the environment in general, specifically in a room, or at the site of a mosquito biting an individual or mammal, for example, on the skin surface.

In each of the methods according to present invention, the vector control management method or control is preferably one or more of a composition, a product and a treated article, each comprising a COMPOUND M.

Preferred further aspects of the present invention are
(i) a product, and a treated article (such as substrates or non-living materials) comprising a compound of any one of the formulae (I), (I-1), (I-2) and (I-A1), preferably a COMPOUND M;
(ii) an integrated mosquito vector management or control solution comprising a compound of any one of the formulae (I), (I-1), (I-2) and (I-A1), preferably a COMPOUND M;
(iii) a method of controlling mosquitoes, preferably mosquito vectors of pathogenic disease, which comprises contacting a mosquito or its environment with a composition comprising a mosquitocidally effective amount of a compound of any one of the formulae (I), (I-1), (I-2) and (I-A1), preferably a COMPOUND M;

In an embodiment, the development of malaria can be reduced by the mosquito control defined in first aspect.

Further, an integrated mosquito vector control management method or control solution comprising one or more compounds of any one of the formulae (I), (I-1), (I-2) and (I-A1), preferably a COMPOUND M, is made available.

In an embodiment, the vector control management method or control solution is a net incorporated with a COMPOUND M; in another embodiment, the vector control management method or control solution is a composition for coating a net, which composition comprises, for example, a COMPOUND M; in further embodiment, the vector control management method or control solution is a composition for spraying surfaces of a dwelling, which composition comprises, for example, a COMPOUND M.

The vector control management method or control solution can comprise a further insecticide and/or synergist.

Another aspect is a polymeric material incorporated with a compound of any one of the formulae (I), (I-1), (I-2) and (I-A1), which material is useful for making substrate or non-living material, such as threads, fibres, yarns, pellets, nets and weaves.

The present invention also makes available
a method of controlling mosquitoes, preferably mosquito vectors of pathogenic disease, with one or more compounds of any one of the formulae (I), (I-1), (I-2) and (I-A1);
a kit for treating a fibre, yarn, net and weave by coating wash resistant insecticidal properties thereto comprising: a first sachet comprising a pre-measured amount of at least one compound of any one of the formulae (I), (I-1), (I-2) and (I-A1), and a second sachet comprising a pre-measured amount of at least one polymeric binder;
a method for treating a fibre, yarn, net and weave by coating wash resistant insecticidal properties thereto comprising (i) preparing a treatment composition, which comprises at least one compound of any one of the formulae (I), (I-1), (I-2) and (I-A1), 2, (ii) treating said fibre, yarn, net and weave and (iii) drying the resulting treated a fibre, yarn, net and weave;
a method of preparing a polymeric material impregnated with a compound of any one of the formulae (I), (I-1), (I-2) and (I-A1), which material is useful for making substrate or non-living material, such as threads, fibres, yarns, pellets, nets and weaves, which method comprises mixing a polymer with the defined compound at a temperature between 120 to 250° C.;
a method for mosquito vector-control, in particular controlling mosquito vectors carrying pathogenic disease, which method comprises (a) applying an effective amount of a liquid composition comprising a compound of any one of the formulae (I), (I-1), (I-2) and (I-A1), and a polymeric binder, and optionally, one or more other insecticides, and/or synergists, to a surface of a dwelling; and/or (b) placing a substrate or non-living material incorporated with a compound of any one of the formulae (I), (I-1), (I-2) and (I-A1), and optionally an additive, one or more other insecticides, and/or synergists, within a dwelling; and
a net incorporated with a compound of any one of the formulae (I), (I-1), (I-2) and (I-A1) having a biological activity in accordance with the WHOPES guidelines of a knockdown after 60 minutes of between 95 percent and 100 percent and/or a mortality after 24 hours of between 80 percent and 100 percent after 20 washes.

In an embodiment, independent of any other embodiments, a compound of formula (I-A1) is a pesticidal compound, preferably an insecticidal compound.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The disclosure in the present application makes available each and every combination of embodiments disclosed herein.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The following Examples serve to illustrate the invention. They do not limit the invention. Temperatures are given in degrees Celsius; mixing ratios of solvents are given in parts by volume.

The compounds of the invention can be distinguished from other similar compounds by virtue of greater efficacy at low application rates, which can be verified by the person skilled in the art using the experimental procedures outlined in the Examples below, using lower concentrations if necessary, for example 10 ppm, 5 ppm, 2 ppm, 1 ppm or 0.2 ppm; or lower application rates, such as 300, 200 or 100, mg of Al per m²

The following abbreviations were used in this section: DMF: dimethylformamide; THF: tetrahydrofuran; EtOAc: ethyl acetate; s=singlet; bs=broad singlet; d=doublet; t=triplet, q=quartet; m=multiplet; Me=methyl; Et=ethyl; Pr=propyl; Bu=butyl; m.p.=melting point.

### PREPARATION EXAMPLES

### Example P1: N-tert-Butyl-5-cyclopropyl-2-methyl-5-phenyl-1,2,4-oxadiazol-3-amine (compound P1.17)

### Step 1: 1-tert-Butyl-3-[cyclopropyl(phenyl)methyl]thiourea

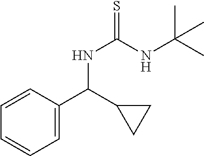 [SMILES: CC(C)(C)NC(=S)NC(c1ccccc1)C1CC1]

Cyclopropyl(phenyl)methanamine (2.00 g) and triethylamine (1.53 g, 2.10 ml) were dissolved in THF (100 ml) and stirred under Argon. tert-Butyl isothiocyanate (3.16 g, 3.48 ml) was slowly added and the reaction mixture was stirred at 66° C. for 18 hours. The reaction mixture was concentrated and the residue was purified by Combiflash with cyclohexane/ethylacetate (0-20%). 3.34 g of 1-tert-butyl-3-[cyclopropyl(phenyl)methyl]thiourea were obtained as a pale beige solid; m.p. 104-107° C. ¹H-NMR [ppm] in CDCl₃: 0.38-0.51 (m, 2H), 0.55-0.68 (m, 2H), 1.13-1.26 (m, 1H), 1.32 (s, 9H), 4.32 (bs, 1H), 5.58 (bs, 1H), 6.26 (bs, 1H); 7.27-7.40 (m, 5H).

### Step 2: N′-tert-Butyl-N-[cyclopropyl(phenyl)methyl]methanediimine

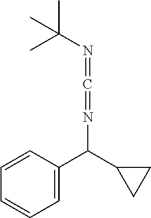 [SMILES: CC(C)(C)N=C=NC(c1ccccc1)C1CC1]

1-tert-Butyl-3-[cyclopropyl(phenyl)methyl]thiourea (2.00 g), bis(2-pyridyloxy)methanethione (1.83 g) were dissolved in acetonitrile (80 ml) and 4-N,N-dimethylamino-pyridine (DMAP, 931 mg) was added. The reaction mixture was stirred under argon at 80° C. for 14 hours (orange solution). The reaction mixture was concentrated. The residue was purified by Combiflash with cyclohexane/ethylacetate. 326 mg of N′-tert-butyl-N-[cyclopropyl(phenyl)methyl]methanediimine were isolated as a colourless liquid. ¹H-NMR [ppm] in CDCl₃: 0.33-0.41 (m, 1H), 0.49-0.58 (m, 2H), 0.64-0.72 (m, 1H), 1.20 (s, 9H), 1.24-1.32 (m, 1H), 3.81 (d, 1H), 7.22-7.40 (m, 5H).

### Step 3: 3-tert-Butyl-2-[cyclopropyl(phenyl)methyl]-1-hydroxy-1-methyl-guanidine hydrochloride

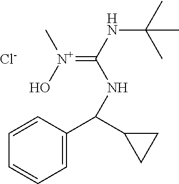 [SMILES: [Cl-] ; C/[N+](O)=C(/NC(c1ccccc1)C1CC1)NC(C)(C)C]

N′-tert-Butyl-N-[cyclopropyl(phenyl)methyl]methanediimine (600 mg) and pyridine (252 mg, 0.258 ml) were dissolved in ethanol (30 ml) and stirred under argon. N-methylhydroxylamine hydrochloride (285 mg) was slowly added and the reaction mixture was stirred at 80° C. for 16 hours. The reaction mixture was concentrated. The residue was purified by Combiflash using dichloromethane/MeOH (0 to 10%) as eluents. 741 mg of 3-tert-butyl-2-[cyclopropyl-(phenyl)methyl]-1-hydroxy-1-methyl-guanidine hydrochloride were obtained as a colourless sticky solid.

### Step 4: N-tert-Butyl-5-cyclopropyl-2-methyl-5-phenyl-1,2,4-oxadiazol-3-amine (compound P1.17)

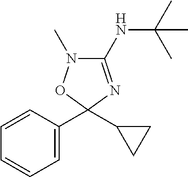 [SMILES: CN1OC(c2ccccc2)(C2CC2)N=C1NC(C)(C)C]

3-tert-Butyl-2-[cyclopropyl(phenyl)methyl]-1-hydroxy-1-methyl-guanidine hydrochloride (628 mg) was dissolved in dichloromethane (20 ml) and stirred at room temperature under Argon. Manganese dioxide (389 mg) was slowly added and the reaction mixture was stirred at room temperature for 17 hours. The reaction mixture was filtered on Celite and washed with dichloromethane. The filtrate was concentrated. The residue was purified by Combiflash with cyclohexane/ethylacetate (0 to 15%). 419 mg of N-tert-butyl-5-cyclopropyl-2-methyl-5-phenyl-1,2,4-oxadiazol-3-amine were isolated as a colourless sticky solid; m.p. 76-78° C. ¹H-NMR [ppm] in CDCl₃: 0.40-0.59 (m, 3H), 0.60-0.67 (m, 1H), 1.34-1.43 (m, 1H), 1.38 (s, 9H), 2.83 (s, 3H), 3.51 (bs, 1H), 7.20-7.27 (m, 1H), 7.28-7.33 (m, 2H), 7.55-7.60 (m, 2H).

### Example P2: N-tert-Butyl-5-cyclopropyl-N,2-dimethyl-5-phenyl-1,2,4-oxadiazol-3-amine (compound P1.18)

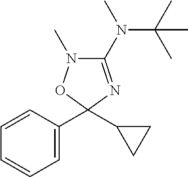 [SMILES: CN1OC(c2ccccc2)(C2CC2)N=C1N(C)C(C)(C)C]

To a suspension of sodium hydride (44 mg) in N,N-dimethylformamide (4 ml) was added N-tert-butyl-5-cyclopropyl-2-methyl-5-phenyl-1,2,4-oxadiazol-3-amine (100 mg) in DMF (0.5 ml). The reaction mixture was stirred at room temperature under argon and iodomethane (156 mg, 0.0686 ml) in DMF (0.5 ml) was added dropwise. The reaction mixture was stirred at room temperature for 4.5 hours. The reaction mixture was poured into cold water (25 ml) and extracted with ethylacetate (2 times). The combined organic phases were washed with water, dried with Na₂SO₄, filtered and concentrated. The residue was purified by Combiflash with cyclohexane/ethylacetate (0 to 10%). 87 mg of N-tert-butyl-5-cyclopropyl-N,2-dimethyl-5-phenyl-1,2,4-oxadiazol-3-amine were isolated as a pale yellow sticky solid. ¹H-NMR [ppm] in CDCl₃: 0.41-0.55 (m, 3H), 0.56-0.62 (m, 1H), 1.32-1.40 (m, 1H), 1.39 (s, 9H), 2.77 (s, 3H), 2.79 (s, 3H), 7.20-7.27 (m, 1H), 7.28-7.33 (m, 2H), 7.53-7.59 (m, 2H).

### Example P3: 5-Cyclopropyl-5-(4-fluorophenyl)-2-methyl-N-(2,2,2-trifluoro-1,1-dimethyl-ethyl)-1,2,4-oxadiazol-3-amine (compound P1.88)

### Step 1: 1-[cyclopropyl-(4-fluorophenyl)methyl]-3-(2,2,2-trifluoro-1,1-dimethyl-ethyl)thiourea

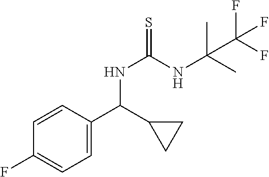 [SMILES: CC(C)(NC(=S)NC(c1ccc(F)cc1)C1CC1)C(F)(F)F]

To a stirred solution of cyclopropyl-(4-fluorophenyl)methanamine (5.00 g) in chloroform (25 ml) was added triethylamine (10.5 ml) followed by the addition of thiophosgene (2.44 g) at 0° C. After the addition the temperature of the reaction mixture was slowly raised to room temperature and stirring was continued for 2 hours. Then 1,1,1-trifluoro-2-methyl-propan-2-amine (3.5 ml) was added at room temperature and stirring was continued for 24 hours. The reaction mixture was purified directly by silica-gel column chromatography using ethyl acetate/hexane as an eluent to give 1.5 g of 1-[cyclopropyl-(4-fluorophenyl)methyl]-3-(2,2,2-trifluoro-1,1-dimethyl-ethyl)thiourea. ¹H-NMR [ppm] in CDCl₃: 0.36-0.48 (m, 2H), 0.55-0.68 (m, 2H), 1.10-1.21 (m, 1H), 1.58 (s, 3H), 1.65 (s, 1H); 4.49 (bs, 1H), 5.72 (bs, 1H), 6.54 (bs, 1H), 7.01-7.08 (m, 2H), 7.28-7.37 (m, 2H).

### Step 2: N-[Cyclopropyl-(4-fluorophenyl)methyl]-N′-(2,2,2-trifluoro-1,1-dimethyl-ethyl)methane-diimine

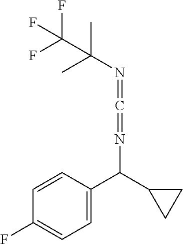 [SMILES: CC(C)(N=C=NC(c1ccc(F)cc1)C1CC1)C(F)(F)F]

To a stirred solution of 1-[cyclopropyl-(4-fluorophenyl)methyl]-3-(2,2,2-trifluoro-1,1-dimethyl-ethyl)thiourea (8.3 g) in dichloromethane (90 ml) at 0° C. were added triethylamine (15.1 ml) and 2-chloro-1,3-dimethyl-4,5-dihydroimidazol-1-ium chloride (6.87 g). After addition the temperature of the reaction mixture was slowly raised to room temperature and stirring was continued for 2 hours. After completion of the reaction the mixture was concentrated under reduce pressure. The residue was purified by silica gel column chromatography using 3% ethyl acetate-hexane as an eluent to give 3.87 g of N-[cyclopropyl-(4-fluorophenyl)methyl]-N′-(2,2,2-trifluoro-1,1-dimethyl-ethyl)methane-diimine as a colorless liquid. ¹H-NMR [ppm] in CDCl₃: 0.31-0.42 (m, 1H), 0.46-0.62 (m, 2H), 0.65-0.76 (m, 1H), 1.19-1.29 (m, 1H), 1.37 (s, 6H), 3.87 (d, 1H), 7.96-7.08 (m, 2H), 7.28-7.40 (m, 2H).

### Step 3: [[[Cyclopropyl-(4-fluorophenyl)methyl]amino]-[(2,2,2-trifluoro-1,1-dimethyl-ethyl)amino]-methylene]-hydroxy-methyl-ammonium chloride

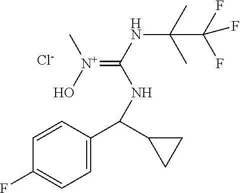 [SMILES: C/[N+](O)=C(/NC(c1ccc(F)cc1)C1CC1)NC(C)(C)C(F)(F)F ; [Cl-]]

To a stirred solution of N-[cyclopropyl-(4-fluorophenyl)methyl]-N′-(2,2,2-trifluoro-1,1-dimethyl-ethyl)methane-diimine (0.8 g) in ethanol (8 ml) was added at room temperature N-methylhydroxylamine hydrochloride (0.29 g) and stirring was continued for 16 hours. After completion of the reaction the reaction mixture was concentrated under reduce pressure to give 830 mg of crude material of [[[cyclopropyl-(4-fluorophenyl)methyl]amino]-[(2,2,2-trifluoro-1,1-dimethyl-ethyl)amino]-methylene]-hydroxy-methyl-ammonium chloride. The crude material was used for the next step without further purification.

### Step 4: 5-Cyclopropyl-5-(4-fluorophenyl)-2-methyl-N-(2,2,2-trifluoro-1,1-dimethyl-ethyl)-1,2,4-oxadiazol-3-amine (compound P1.88)

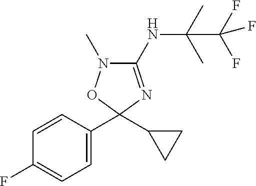 [SMILES: CN1OC(c2ccc(F)cc2)(C2CC2)N=C1NC(C)(C)C(F)(F)F]

To a stirred solution of crude material of [[[cyclopropyl-(4-fluorophenyl)methyl]amino]-[(2,2,2-trifluoro-1,1-dimethyl-ethyl)amino]-methylene]-hydroxy-methyl-ammonium chloride (2.4 g) in THF (25 ml) was added MnO₂ (3.18 g) at room temperature and stirring was continued for 4 h. After completion of the reaction the reaction mixture was passed through a celite-bed and the collected solvent was concentrated under reduced pressure to give the crude compound. The crude compound was purified by silica gel column chromatography using hexane-ethyl acetate (2-20%) as an eluent to give 41 mg of 5-cyclopropyl-5-(4-fluorophenyl)-2-methyl-N-(2,2,2-trifluoro-1,1-dimethyl-ethyl)-1,2,4-oxadiazol-3-amine as an off-white solid; 76-78° C. ¹H-NMR [ppm] in CDCl₃: 0.42-0.61 (m, 4H), 1.29-1.38 (m, 1H), 1.62 (s, 3H), 1.63 (s, 3H), 2.86 (s, 3H), 3.74 (bs, 1H), 7.95-7.05 (m, 2H), 7.44-7.55 (m, 2H).

### Example P4: N-tert-Butyl-5-ethyl-2-methyl-5-phenyl-1,2,4-oxadiazol-3-amine (compound P1.94)

### Step 1: 1-Phenylpropylidenecyanamide

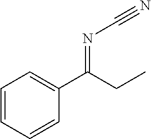

To a stirring solution of 1-phenylpropan-1-one (4 g) in dichloromethane (30 ml) was added TiCl₄ (1M in dichloromethane, 74.5 ml) at 0° C. After addition the temperature of the reaction mixture was slowly raised to room temperature and stirring was continued for 1 hour. The reaction mixture was cooled again and a solution of N,N′-bis(trimethylsilyl)methanediimine (12.2 g) in dichloromethane was added. Then the temperature of the reaction was slowly raised to room temperature and stirring was continued for 1 hour. After completion of the reaction the mixture was quenched with water and extracted with dichloromethane. The combined organic layers were dried over sodium sulfate, filtered and concentrated under reduced pressure to give 4.5 g of crude material of 1-phenylpropylidenecyanamide. This material was used for the next step without further purification.

### Step 2: 5-Ethyl-2-methyl-5-phenyl-1,2,4-oxadiazol-3-amine

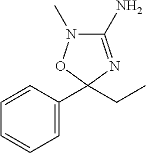

To a stirred solution of crude 1-phenylpropylidenecyanamide (950 mg) in dry THF (8 ml) were added triethylamine (1.76 ml) and molecular sieves. The reaction mixture was stirred for 30 minutes at room temperature. Then a solution of hydroxylamine hydrochloride (2 g) in THF was added to the reaction mixture at room temperature and stirring was continued for 1 hour. The solid was filtered off and the solvent was evaporated to dryness. A sticky mass was obtained which was triturated with pentane. The solvent was decanted and the process was repeated twice. The combined solid was dried under vacuum to give 1.9 g of 5-ethyl-2-methyl-5-phenyl-1,2,4-oxadiazol-3-amine. ¹H-NMR [ppm] in d₆-DMSO: 0.77 (t, 3H), 1.71-1.83 (m, 2H), 2.85 (s, 3H), 3.32 (bs, 1H), 6.05 (s, 2H), 7.21 (t, 1H), 7.29 (t, 2H), 7.40 (d, 2H).

### Step 3: N-tert-butyl-5-ethyl-2-methyl-5-phenyl-1,2,4-oxadiazol-3-amine (compound P1.94)

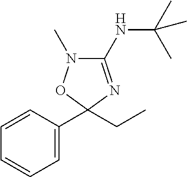 [SMILES: CCC1(c2ccccc2)N=C(NC(C)(C)C)N(C)O1]

To a stirred solution of 5-ethyl-2-methyl-5-phenyl-1,2,4-oxadiazol-3-amine (250 mg) and tert-butyl 2,2,2-trichloroethanimidate (0.87 ml) in THF:n-hexane (1:1, 3 ml) was added BF₃-Et₂O at 0° C. After addition the temperature of the reaction was slowly raised to room temperature and stirring was continued for 16 hours. The reaction was quenched with saturated aqueous NaHCO₃-solution and extracted with ethyl acetate (3 times). The combined organic layers were dried over sodium sulfate, filtered and concentrated under reduced pressure. The residue was purified by column chromatography using hexane-ethyl acetate (20%) to give 23 mg of N-tert-butyl-5-ethyl-2-methyl-5-phenyl-1,2,4-oxadiazol-3-amine as a solid; 48-50° C. ¹H-NMR [ppm] in CDCl₃: 0.90 (t, 3H), 1.42 (s, 9H), 1.93 (q, 2H), 2.80 (s, 3H), 3.49 (bs, 1H), 7.12-7.32 (m, 3H), 7.44-7.55 (m, 2H).

### Example P5: N-tert-Butyl-5,5-bis(4-fluorophenyl)-3-methyl-4H-imidazol-2-amine (compound P2.1)

### Step 1: 5,5-Bis(4-fluorophenyl)-3-methyl-2-thioxo-imidazolidin-4-one

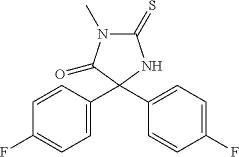 [SMILES: CN1C(=O)C(c2ccc(F)cc2)(c2ccc(F)cc2)NC1=S]

1,2-Bis(4-fluorophenyl)ethane-1,2-dione (2.00 g) and methylthiourea (3.30 g) were dissolved in ethanol (40 ml) and 1,4-dioxane (40 mL). The reaction mixture was stirred at room temperature and sodium carbonate (3.87 g) in water (20 ml) was added dropwise. The reaction mixture was then stirred at 85° C. for 5 hours. Then the reaction mixture was concentrated and diluted with ethylacetate (100 ml) and washed with brine (20 ml). The organic phase was washed with brine, dried with magnesium sulfate, filtered and concentrated. The residue was purified by Combiflash with cyclohexane/ethylacetate (0 to 20%) to give 2.38 g of 5,5-bis(4-fluorophenyl)-3-methyl-2-thioxo-imidazolidin-4-one as a solid; m.p. 161-163° C. ¹H-NMR [ppm] in CDCl₃: 3.30 (s, 3H), 7.01-7.09 (m, 4H), 7.25-7.32 (m, 4H), 8.56 (bs, 1H).

### Step 2: 5,5-Bis(4-fluorophenyl)-3-methyl-2-methylsulfanyl-imidazol-4-one

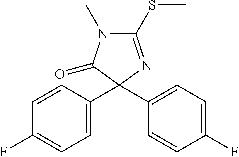 [SMILES: CSC1=NC(c2ccc(F)cc2)(c2ccc(F)cc2)C(=O)N1C]

5,5-Bis(4-fluorophenyl)-3-methyl-2-thioxo-imidazolidin-4-one (400 mg) was dissolved in acetonitrile (13 ml). Potassium carbonate (194 mg) was added and the reaction mixture was stirred at room temperature. Iodomethane (268 mg, 0.117 m) was then added dropwise under argon. The reaction mixture was then stirred at 60° C. for 20 hours. The reaction mixture was concentrated then suspended in dichloromethane. The insoluble salt was filtered off and the filtrate was concentrated. 277 mg of 5,5-bis(4-fluorophenyl)-3-methyl-2-methylsulfanyl-imidazol-4-one were obtained as a solid; m.p. 135-137° C. ¹H-NMR [ppm] in CDCl₃: 2.68 (s, 3H), 3.08 (s, 3H), 6.94-7.02 (m, 4H), 7.46-7.55 (m, 4H).

### Step 3: 2-(tert-Butylamino)-5,5-bis(4-fluorophenyl)-3-methyl-imidazol-4-one

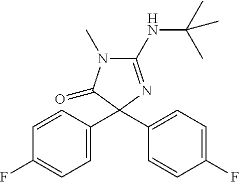 [SMILES: CN1C(=O)C(c2ccc(F)cc2)(c2ccc(F)cc2)N=C1NC(C)(C)C]

5,5-Bis(4-fluorophenyl)-3-methyl-2-methylsulfanyl-imidazol-4-one (989 mg) was dissolved in dichloromethane (30 ml) and cooled down to 0° C. Sulfuryl chloride (1.406 g) was slowly added and the reaction mixture was stirred at room temperature for 19 hours. The reaction mixture was concentrated. tert-Butylamine (6.25 ml), silver nitrate (505 mg) and sodium carbonate (473 mg) were was added to the residue. The reaction mixture was stirred under microwave conditions at 135° C. for 5 hours. The reaction mixture was concentrated, dissolved in tert-butyl methyl ether, and then filtrated trough Celite. The filtrate was concentrated and purified by Combiflash with cyclohexane/EtOAc as eluents (0 to 20%). The fractions containing the desired product were concentrated and further purified by reversed phase preparative HPLC to give 234 mg of 2-(tert-butylamino)-5,5-bis(4-fluorophenyl)-3-methyl-imidazol-4-one as a solid; m.p. 149-150° C. ¹H-NMR [ppm] in CDCl₃: 1.56 (s, 9H), 2.98 (s, 3H), 3.84 (bs, 1H), 6.91-6.98 (m, 4H), 7.58-7.67 (m, 4H).

### Step 4: N-tert-Butyl-5,5-bis(4-fluorophenyl)-3-methyl-4H-imidazol-2-amine (compound P2.1)

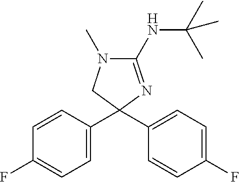 [SMILES: CN1CC(c2ccc(F)cc2)(c2ccc(F)cc2)N=C1NC(C)(C)C]

To a suspension of lithium aluminium hydride (16 mg) in diethylether (0.42 ml) was added a solution of trichloroalumane (56 mg) in diethylether (0.42 ml). The mixture was stirred at room temperature and 2-(tert-butylamino)-5,5-bis(4-fluorophenyl)-3-methyl-imidazol-4-one (50 mg) in diethylether (0.42 ml) and THF (0.46 ml) was slowly added. After 3 hours the reaction was stopped. A saturated solution of sodium carbonate was added and the inorganic residue was filtered. The filtrate was washed with water and concentrated. The residue was purified by Combiflash with hexane/ethylacetate (0-60%) to give 48 mg of N-tert-butyl-5,5-bis(4-fluorophenyl)-3-methyl-4H-imidazol-2-amine as a solid; m.p. 99-102° C. ¹H-NMR [ppm] in CDCl₃: 1.47 (s, 9H), 2.69 (s, 3H), 3.70 (s, 2H), 4.5-4.9 (bs, 1H) 6.90-6.99 (m, 4H), 7.40-7.46 (m, 2H).

### Example P6: 4-[3-(tert-butylamino)-5-cyclopropyl-2-methyl-1,2,4-oxadiazol-5-yl]benzonitrile (compound P1.136)

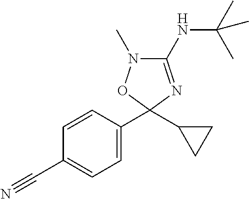 [SMILES: CN1OC(c2ccc(C#N)cc2)(C2CC2)N=C1NC(C)(C)C]

5-(4-bromophenyl)-N-tert-butyl-5-cyclopropyl-2-methyl-1,2,4-oxadiazol-3-amine (150 mg) which was prepared analogously as described above was dissolved in degassed N,N-dimethylformamide (2.73 ml). Dicyanozinc (55 mg) and tetrakis(triphenylphosphine)-palladium (49 mg) were added. The mixture was stirred at 100° C. for 17 hours. The solution was diluted with ethyl acetate and washed with water, dried and concentrated. The residue was purified by Combiflash with Cyclohexane/Ethylacetate (0 to 15%) to give 104 mg of 4-[3-(tert-butylamino)-5-cyclopropyl-2-methyl-1,2,4-oxadiazol-5-yl]benzonitrile as a gum. ¹H-NMR [ppm] in CDCl₃: 0.44-0.56 (m, 3H), 0.60-0.66 (m, 1H), 1.28-1.33 (m, 1H), 1.38 (s, 9H), 2.85 (s, 3H), 3.56 (bs, 1H), 7.56-7.63 (m, 2H), 7.65-7.68 (m, 2H).

### Example P7: N-tert-butyl-5-cyclopropyl-5-[4-(4-fluorophenyl)phenyl]-2-methyl-1,2,4-oxadiazol-3-amine (compound P1.137)

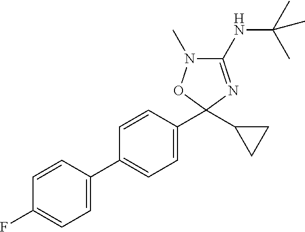 [SMILES: CN1OC(c2ccc(-c3ccc(F)cc3)cc2)(C2CC2)N=C1NC(C)(C)C]

5-(4-bromophenyl)-N-tert-butyl-5-cyclopropyl-2-methyl-1,2,4-oxadiazol-3-amine (160 mg) which was prepared analogously as described above was dissolved in 1,2-dimethoxyethane (2.82 ml). Then (4-fluorophenyl)boronic acid (66.7 mg), disodium carbonate (5.00 eq) in water and tetrakis (triphenylphosphine)-palladium (21.1 mg) were added. The mixture was stirred at 85° C. for 8 hours. The reaction mixture was poured into water and extracted with ethylacetate. The organic phases were washed with brine, dried and concentrated. The residue was purified by Combiflash with Cyclohexane/Ethylacetate (0 to 15%) to give 134 mg of N-tert-butyl-5-cyclopropyl-5-[4-(4-fluorophenyl)phenyl]-2-methyl-1,2,4-oxadiazol-3-amine as a gum. ¹H-NMR [ppm] in CDCl₃: 0.45-0.58 (m, 3H), 0.62-0.68 (m, 1H), 1.39-1.48 (m, 1H), 1.41 (s, 9H), 2.87 (s, 3H), 3.57 (bs, 1H), 7.09 (t, 2H), 7.47-7.56 (m, 4H), 7.63 (d, 2H).

The compounds in Table P1 and P2 were prepared as described in the examples above or similar methodology.

TABLE P1
Compounds of formula (I-1)

| | |
|---|---|
| 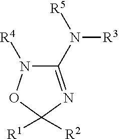 | (I-1) |

The compounds in Table P1 can be prepared as described in the examples above or similar methodology. The following abbreviations are used in the table below: Me=methyl, Et=ethyl, Pr=propyl, Bu=butyl, Ph=phenyl, Bn=benzyl, LQ=liquid, GM=gum.

| Cmpd No. | R1 | R2 | R3 | R4 | R5 | Melting Point |
|---|---|---|---|---|---|---|
| P1.1 | 4-F—Ph | Ph | t-Bu | Me | Me | 85-87° C. |
| P1.2 | 4-F—Ph | 4-F—Ph | t-Bu | Me | H | 100-102° C. |
| P1.3 | 4-Cl—Ph | c-Pr | t-Bu | Me | H | 72-73° C. |
| P1.4 | Ph | Ph | t-Bu | Me | H | 131-132° C. |
| P1.5 | 4-F—Ph | Ph | t-Bu | Me | H | 112-114° C. |
| P1.6 | 3-F—Ph | Ph | t-Bu | Me | Et | LQ |
| P1.7 | 4-F—Ph | Ph | t-Bu | Me | Et | LQ |
| P1.8 | 4-F—Ph | Me | t-Bu | Me | H | 84-90° C. |
| P1.9 | 3-Cl—Ph | c-Pr | t-Bu | Me | H | 63-66° C. |
| P1.10 | 3-Cl—Ph | c-Pr | t-Bu | Me | Me | LQ |
| P1.11 | 3-Cl—Ph | c-Pr | t-Bu | Me | Et | LQ |
| P1.12 | 3-CF₃—Ph | Ph | t-Bu | Me | H | 133-136° C. |
| P1.13 | 4-F—Ph | c-Pr | t-Bu | Me | H | 70-72° C. |
| P1.14 | 4-MeO—Ph | c-Pr | t-Bu | Me | H | LQ |
| P1.15 | 4-F—Ph | c-Pr | t-Bu | Me | Me | LQ |
| P1.16 | 4-CF₃—Ph | Ph | t-Bu | Me | Et | LQ |
| P1.17 | Ph | c-Pr | t-Bu | Me | H | 76-78° C. |
| P1.18 | Ph | c-Pr | t-Bu | Me | Me | LQ |
| P1.19 | Ph | c-Pr | t-Bu | Me | Et | LQ |
| P1.20 | 4-MeO—Ph | c-Pr | t-Bu | Me | Me | LQ |
| P1.21 | 4-MeO—Ph | c-Pr | t-Bu | Me | Et | LQ |
| P1.22 | 4-CF₃O—Ph | Ph | t-Bu | Me | H | LQ |
| P1.23 | 4-CF₃O—Ph | Ph | t-Bu | Me | Me | LQ |
| P1.24 | 4-CF₃O—Ph | Ph | t-Bu | Me | Et | LQ |
| P1.25 | 4-F—Ph | 4-F—Ph | t-Bu | Et | Me | 92-96° C. |
| P1.26 | 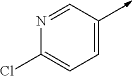 | Me | t-Bu | Me | H | LQ |
| P1.27 | 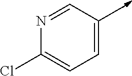 | Me | t-Bu | Me | Me | LQ |
| P1.28 | 3-F—Ph | c-Pr | t-Bu | Me | H | GM |
| P1.29 | 4-CF₃O—Ph | c-Pr | t-Bu | Me | H | GM |
| P1.30 | 3-F—Ph | c-Pr | t-Bu | Me | Me | LQ |
| P1.31 | 4-CF₃O—Ph | c-Pr | t-Bu | Me | Me | LQ |
| P1.32 | 4-CF₃—Ph | c-Pr | t-Bu | Me | H | GM |
| P1.33 | 4-CF₃—Ph | c-Pr | t-Bu | Me | Me | GM |
| P1.34 | 3,4-Cl₂—Ph | c-Pr | t-Bu | Me | H | 96-98° C. |
| P1.35 | 3,4-F₂—Ph | c-Pr | t-Bu | Me | H | 55-57° C. |
| P1.36 | 3,5-Cl₂—Ph | c-Pr | t-Bu | Me | H | 126-129° C. |
| P1.37 | 3,4-Cl₂—Ph | c-Pr | t-Bu | Me | Me | LQ |
| P1.38 | 3,4-F₂—Ph | c-Pr | t-Bu | Me | Me | LQ |
| P1.39 | 3,5-Cl₂—Ph | c-Pr | t-Bu | Me | Me | LQ |
| P1.40 | 4-F—Ph | H | t-Bu | Me | H | GM |
| P1.41 | 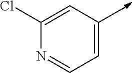 | Me | t-Bu | Me | Me | 58-60° C. |
| P1.42 | 2-F—Ph | c-Pr | t-Bu | Me | H | SO |
| P1.43 | 4-t-Bu—Ph | c-Pr | t-Bu | Me | H | 110-112° C. |
| P1.44 | 2-F—Ph | c-Pr | t-Bu | Me | Me | 62-63° C. |
| P1.45 | 4-t-Bu—Ph | c-Pr | t-Bu | Me | Me | GM |
| P1.46 | 4-F—Ph | i-Pr | t-Bu | Me | H | 98-100° C. |
| P1.47 | 4-F—Ph | Ph—CH₂CH₂ | t-Bu | Me | H | GM |
| P1.48 | 4-F—Ph | c-Pentyl | t-Bu | Me | H | 88-91° C. |
| P1.49 | 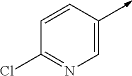 | c-Pr | t-Bu | Me | Me | LQ |
| P1.50 | 3-CF₃—Ph | Ph | t-Bu | Me | H | 95-96° C. |
| P1.51 | 3-CF₃—Ph | Ph | t-Bu | Me | Me | 92-93° C. |
| P1.52 | 4-Br—Ph | c-Pr | t-Bu | Me | H | GM |
| P1.53 | 4-Br—Ph | c-Pr | t-Bu | Me | Me | LQ |
| P1.54 | 4-c-Pr—Ph | c-Pr | t-Bu | Me | H | LQ |
| P1.55 | 3-CF₃—Ph | c-Pr | t-Bu | Me | H | 51-52° C. |
| P1.56 | 2-Cl—Ph | c-Pr | t-Bu | Me | H | 96-98° C. |
| P1.57 | 3-CF₃—Ph | c-Pr | t-Bu | Me | Me | GM |
| P1.58 | 2-Cl—Ph | c-Pr | t-Bu | Me | Me | 68-70° C. |
| P1.59 | 2,4-Cl₂—Ph | c-Pr | t-Bu | Me | H | LQ |
| P1.60 | 2,4-F₂—Ph | c-Pr | t-Bu | Me | H | LQ |
| P1.61 | 2,3-Cl₂—Ph | c-Pr | t-Bu | Me | H | 96-98° C. |
| P1.62 | 2,4-Cl₂—Ph | c-Pr | t-Bu | Me | Me | LQ |
| P1.63 | 2,4-F₂—Ph | c-Pr | t-Bu | Me | Me | LQ |
| P1.64 | 2,3-Cl₂—Ph | c-Pr | t-Bu | Me | Me | GM |
| P1.65 | 2,3-F₂—Ph | c-Pr | t-Bu | Me | H | 78-80° C. |
| P1.66 | 2,3-F₂—Ph | c-Pr | t-Bu | Me | Me | GM |
| P1.67 | 2,6-F₂—Ph | c-Pr | t-Bu | Me | H | GM |
| P1.68 | 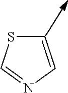 | c-Pr | t-Bu | Me | Me | 80-82° C. |
| P1.69 | 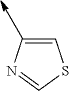 | c-Pr | t-Bu | Me | Me | LQ |
| P1.70 | 4-F—Ph | c-Pr—CH₂ | t-Bu | Me | H | LQ |
| P1.71 | 4-F—Ph | c-Pr—CH₂ | t-Bu | Me | Et | 112-114° C. |
| P1.72 | 3-Br—Ph | c-Pr | t-Bu | Me | H | 76-78° C. |
| P1.73 | 3-CN—Ph | c-Pr | t-Bu | Me | H | GM |
| P1.74 | 3-c-Pr—Ph | c-Pr | t-Bu | Me | H | GM |
| P1.75 | 3-Br—Ph | c-Pr | t-Bu | Me | Me | GM |
| P1.76 | 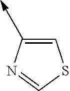 | c-Pr | t-Bu | Me | H | 116-118° C. |
| P1.77 | 4-F—Ph | c-Pr | 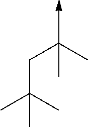 | Me | H | 40-42° C. |
| P1.78 | 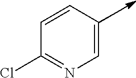 | c-Pr | t-Bu | Me | H | 58-60° C. |
| P1.79 | 4-F—Ph | c-Pr | 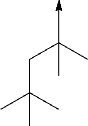 | Me | Me | 59-61° C. |
| P1.80 | 4-F—Ph | c-Pr | CMe₂Et | Me | H | 42-44° C. |
| P1.81 | 4-F—Ph | c-Pr | CMe₂Et | Me | Me | LQ |
| P1.82 | 2,4,6-F₃—Ph | c-Pr | t-Bu | Me | H | GM |
| P1.83 | 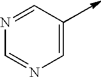 | c-Pr | t-Bu | Me | Me | GM |
| P1.84 | 4-F—Ph | c-Bu | t-Bu | Me | H | 70-72° C. |
| P1.85 | 4-F—Ph | c-Bu | t-Bu | Me | Me | 42-44° C. |
| P1.86 | Ph | c-Bu | t-Bu | Me | H | GM |
| P1.87 | Ph | c-Bu | t-Bu | Me | Me | GM |
| P1.88 | 4-F—Ph | c-Pr | 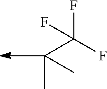 | Me | H | 76-78° C. |
| P1.89 | 4-F—Ph | c-Pr | 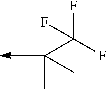 | Me | Me | LQ |
| P1.90 | 4-F—Ph | c-Pr | 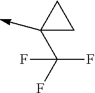 | Me | H | 88-90° C. |
| P1.91 | 4-F—Ph | c-Pr | 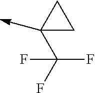 | Me | Me | LQ |
| P1.92 | 4-F—Ph | c-Pr | 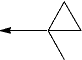 | Me | H | LQ |
| P1.93 | 4-F—Ph | c-Pr | 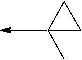 | Me | Me | 42-44° C. |
| P1.94 | Ph | Et | t-Bu | Me | H | 48-50° C. |
| P1.95 | Ph | Et | t-Bu | Me | Me | LQ |
| P1.96 | 4-F—Ph | c-Pr | CF₃—CH₂ | Me | H | 80-84°C. |
| P1.97 | 4-F—Ph | c-Pr | CF₃—CH₂ | Me | Me | LQ |
| P1.98 | Ph | n-Pr | t-Bu | Me | H | 62-64° C. |
| P1.99 | 4-F—Ph | Et | t-Bu | Me | H | 58-60° C. |
| P1.100 | 4-F—Ph | n-Pr | t-Bu | Me | H | 38-40° C. |
| P1.101 | 4-F—Ph | n-Pr | t-Bu | Me | Me | LQ |
| P1.102 | Ph | 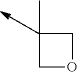 | t-Bu | Me | H | 124-126° C. |
| P1.103 | 4-F—Ph | 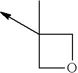 | t-Bu | Me | Me | GM |
| P1.104 | 4-F—Ph | c-Pr | CEt₃ | Me | H | 68-70° C. |
| P1.105 | 4-F—Ph | c-Pr | CEt₃ | Me | Me | 41-43° C. |
| P1.106 | 4-F—Ph | c-Pr | CMeEt₂ | Me | H | 40-42° C. |
| P1.107 | 4-F—Ph | c-Pr | CMeEt₂ | Me | Me | LQ |
| P1.108 | 4-F—Ph | c-Pr | 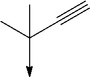 | Me | H | 68-70° C. |
| P1.109 | 4-Cl—Ph | 4-Cl—Ph | t-Bu | Me | Et | LQ |
| P1.110 | 4-Cl—Ph | 4-Cl—Ph | t-Bu | Me | Me | LQ |
| P1.111 | Bn | Ph | t-Bu | Me | Me | LQ |
| P1.112 | 4-F—Ph | Me | t-Bu | Me | Me | LQ |
| P1.113 | 4-F—Ph | c-Pr | t-Bu | Me | Et | LQ |
| P1.114 | 4-F—Ph | 4-F—Ph | t-Bu | Allyl | H | 80-83° C. |
| P1.115 | 3-F—Ph | 3-F—Ph | t-Bu | Me | Me | GM |
| P1.116 | 4-Cl—Ph | CF₃ | t-Bu | Me | H | GM |
| P1.117 | 4-F—Ph | 4-F—Ph | t-Bu | Et | H | 78-81° C. |
| P1.118 | 4-F—Ph | 4-F—Ph | t-Bu | Et | Et | 72-76° C. |
| P1.119 | 3,5-Cl₂—Ph | CF₃ | t-Bu | Me | H | GM |
| P1.120 | 1-Naphtyl | 4-F—Ph | t-Bu | Me | H | 68-70° C. |
| P1.121 | 1-Naphtyl | 4-F—Ph | t-Bu | Me | Me | 108-110° C. |
| P1.122 | 1-Naphtyl | 4-F—Ph | t-Bu | Me | Et | GM |
| P1.123 | 2-Naphtyl | 4-F—Ph | t-Bu | Me | Et | LQ |
| P1.124 | 4-Cl—Ph | Ph | t-Bu | Me | Me | 75-77° C. |
| P1.125 | 3-Cl—Ph | Ph | t-Bu | Me | Me | 85-87° C. |
| P1.126 | 4-F—Ph | 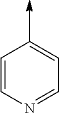 | t-Bu | Me | Me | 70-72° C. |
| P1.127 | 4-F—Ph | 4-F—Ph | Allyl | Me | H | 78-80° C. |
| P1.128 | 2-Naphtyl | 4-F—Ph | t-Bu | Me | H | GM |
| P1.129 | 2-Naphtyl | 4-F—Ph | t-Bu | Me | Me | GM |
| P1.130 | 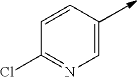 | c-Pr | t-Bu | Me | H | 72-74° C. |
| P1.131 | 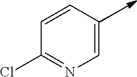 | Ph | t-Bu | Me | Me | 108-110° C. |
| P1.132 | 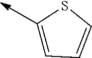 | Ph | t-Bu | Me | Me | 86-88° C. |
| P1.133 | 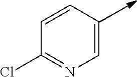 | Ph | t-Bu | Me | H | 112-114° C. |
| P1.134 | 4-F—Ph | Ph—CH₂CH₂ | t-Bu | Me | Et | LQ |
| P1.135 | 4-MeS—Ph | c-Pr | t-Bu | Me | H | GM |
| P1.136 | 4-CN—Ph | c-Pr | t-Bu | Me | H | GM |
| P1.137 | 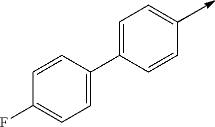 | c-Pr | t-Bu | Me | H | LQ |
| P1.138 | 4-F—Ph | 4-F—Ph | 4-Cl—Ph | Me | H | 112-114° C. |
| P1.139 | 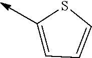 | 4-F—Ph | t-Bu | Me | H | 100-102° C. |
| P1.140 | 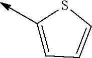 | 4-F—Ph | t-Bu | Me | Me | 64-66° C. |
| P1.141 | 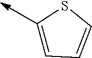 | c-Pr | t-Bu | Me | H | 102-104° C. |
| P1.142 | 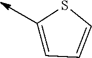 | c-Pr | t-Bu | Me | Me | LQ |
| P1.143 | 2,6-F₂—Ph | c-Pr | t-Bu | Me | Me | GM |
| P1.144 | 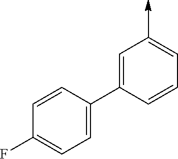 | c-Pr | t-Bu | Me | H | GM |
| P1.145 | 4-F—Ph | c-Pr | 1-adamantyl | Me | H | 108-114° C. |
| P1.146 | 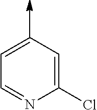 | c-Pr | t-Bu | Me | Me | 48-52° C. |
| P1.147 | 4-F—Ph | c-Pr | Ph | Me | H | 128-130° C. |
| P1.148 | Ph | CF₃—CH₂— | t-Bu | Me | H | 61-63° C. |
| P1.149 | Ph | CF₃—CH₂— | t-Bu | Me | Me | LQ |
| P1.150 | 4-F—Ph | Et | t-Bu | Me | Me | LQ |
| P1.151 | 4-F—Ph | c-Pr | c-Pr | Me | H | 104-108° C. |
| P1.152 | 4-F—Ph | c-Pr | 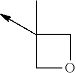 | Me | H | GM |
| P1.153 | 4-F—Ph | c-Pr | 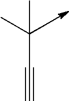 | Me | Me | LQ |
| P1.154 | 2-F—Ph | Et | t-Bu | Me | Me | LQ |
| P1.155 | 2-F—Ph | Et | t-Bu | Me | H | LQ |
| P1.156 | 4-F—Ph | c-Pr | t-Bu | Et | H | LQ |
| P1.157 | c-Pr | c-Pr | t-Bu | Me | H | 88-90° C. |
| P1.158 | 3-Cl—Ph | Et | t-Bu | Me | H | LQ |
| P1.159 | 3-Cl—Ph | Et | t-Bu | Me | Me | LQ |
| P1.160 | 4-F—Ph | c-Pr | Et | Me | H | LQ |
| P1.161 | 4-F—Ph | c-Pr | i-Pr | Me | H | 42-44° C. |
| P1.162 | Ph | n-Bu | t-Bu | Me | Me | LQ |
| P1.163 | 4-F—Ph | n-Bu | t-Bu | Me | Me | LQ |
| P1.164 | 4-F—Ph | n-Bu | t-Bu | Me | H | LQ |
| P1.165 | 2-Cl—Ph | n-Pr | t-Bu | Me | H | SO |
| P1.166 | 3-F—Ph | n-Pr | t-Bu | Me | Me | LQ |
| P1.167 | 3-F—Ph | n-Pr | t-Bu | Me | H | 38-40° C. |
| P1.168 | 2-F—Ph | n-Pr | t-Bu | Me | Me | LQ |
| P1.169 | 2-F—Ph | n-Pr | t-Bu | Me | H | LQ |
| P1.170 | 3-Cl—Ph | n-Pr | t-Bu | Me | Me | 39-40° C. |
| P1.171 | 3-Cl—Ph | n-Pr | t-Bu | Me | H | 50-52° C. |
| P1.172 | 4-Cl—Ph | Et | t-Bu | Me | Me | 37-38° C. |
| P1.173 | 4-Cl—Ph | Et | t-Bu | Me | H | 80-82° C. |
| P1.174 | 2,3-F₂—Ph | Et | t-Bu | Me | H | 66-68° C. |
| P1.175 | 2,3-F₂—Ph | Et | t-Bu | Me | Me | LQ |
| P1.176 | 3-F—Ph | Et | t-Bu | Me | H | LQ |
| P1.177 | 3-F—Ph | Et | t-Bu | Me | Me | LQ |
| P1.178 | 2,3-F₂—Ph | n-Pr | t-Bu | Me | Me | LQ |
| P1.179 | 4-Cl—Ph | n-Pr | t-Bu | Me | Me | 38-40° C. |
| P1.180 | 4-Cl—Ph | n-Pr | t-Bu | Me | H | 50-52° C. |
| P1.181 | 2,3-F₂—Ph | n-Pr | t-Bu | Me | H | LQ |
| P1.182 | 4-OMe—Ph | n-Pr | t-Bu | Me | H | LQ |
| P1.183 | 4-OMe—Ph | n-Pr | t-Bu | Me | Me | LQ |
| P1.184 | 2-Cl—Ph | Et | t-Bu | Me | H | 76-78° C. |
| P1.185 | 2-Cl—Ph | Et | t-Bu | Me | Me | LQ |
| P1.186 | Ph | n-pentyl | t-Bu | Me | H | LQ |
| P1.187 | Ph | Me | CMe₂Et | Me | H | LQ |
| P1.188 | 4-F—Ph | n-pentyl | t-Bu | Me | H | LQ |
| P1.189 | 4-F—Ph | n-pentyl | t-Bu | Me | Me | LQ |
| P1.190 | 2-Me—Ph | n-Pr | t-Bu | Me | H | 71-72° C. |
| P1.191 | 4-Me—Ph | n-Pr | t-Bu | Me | H | LQ |
| P1.192 | Ph | n-Pr | CMe₂Et | Me | H | 58-60° C. |
| P1.193 | Ph | n-pentyl | t-Bu | Me | Me | LQ |
| P1.194 | 3-Me—Ph | n-Pr | t-Bu | Me | H | LQ |
| P1.195 | 4-Me—Ph | n-Pr | t-Bu | Me | Me | LQ |
| P1.196 | 3-OMe—Ph | n-Pr | t-Bu | Me | Me | LQ |
| P1.197 | 3-OMe—Ph | n-Pr | t-Bu | Me | H | Gum |
| P1.198 | 2-OMe—Ph | n-Pr | t-Bu | Me | Me | Gum |
| P1.199 | 2-OMe—Ph | n-Pr | t-Bu | Me | H | 64-66° C. |
| P1.200 | Ph | n-Pr | 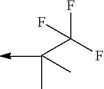 | Me | H | LQ |
| P1.201 | Ph | n-Bu | 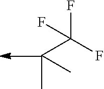 | Me | H | LQ |
| P1.202 | Ph | Me | 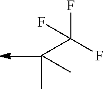 | Me | H | 82-84° C. |
| P1.203 | Ph | 4-F—Ph— | t-Bu | Me | H | LQ |
| | | CH₂CH₂— | | | | |
| P1.204 | 3-Me—Ph | n-Pr | t-Bu | Me | H | LQ |
| P1.205 | Ph | Et | CMe₂Et | Me | H | 48-50° C. |
| P1.206 | Ph | n-Bu | t-Bu | Me | H | 44-46° C. |
| P1.207 | Ph | n-Bu | CMe₂Et | Me | H | LQ |
| P1.208 | 4-Br—Ph | n-Bu | t-Bu | Me | H | 48-50° C. |
| P1.209 | Ph | allyl | t-Bu | Me | H | LQ |
| P1.210 | 4-OMe—Ph | n-Bu | t-Bu | Me | H | LQ |
| P1.211 | 4-F—Ph | allyl | t-Bu | Me | H | LQ |
| P1.212 | 4-F—Ph | Me | 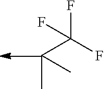 | Me | H | 80-82° C. |
| P1.213 | 4-F—Ph | Me | CMe₂Et | Me | H | 60-62° C. |
| P1.214 | 4-Me—Ph | n-Bu | t-Bu | Me | H | Gum |
| P1.215 | 4-F—Ph | Et | CMe₂Et | Me | H | 48-50° C. |
| P1.216 | 4-F—Ph | Et | 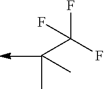 | Me | H | 96-97° C. |
| P1.217 | Ph | n-Hex | t-Bu | Me | H | LQ |
| P1.218 | 4-F—Ph | n-Pr | t-Bu | Et | H | LQ |
| P1.219 | Ph | 4-Cl—Ph— | t-Bu | Me | H | LQ |
| | | CH₂CH₂— | | | | |
| P1.220 | 4-F—Ph | n-Bu | 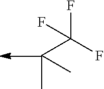 | Me | H | LQ |
| P1.221 | 4-F—Ph | n-Bu | t-Bu | Et | H | LQ |
| P1.222 | Ph | n-Bu | t-Bu | Et | H | LQ |
| P1.223 | 4-Cl—Ph | n-Bu | t-Bu | Me | H | LQ |
| P1.224 | 4-F—Ph | n-Pr | 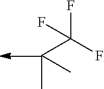 | Me | H | LQ |
| P1.225 | 4-F—Ph | Me | t-Bu | Et | H | 51-53° C. |
| P1.226 | Ph | Et | t-Bu | n-Bu | H | LQ |
| P1.227 | 4-F—Ph | Et | t-Bu | Et | H | 70-72° C. |
| P1.228 | Ph | n-Pr | t-Bu | Et | H | 65-67° C. |
| P1.229 | Ph | n-Pr | t-Bu | n-Pr | H | LQ |
| P1.230 | 4-F—Ph | n-Hex | t-Bu | Me | H | LQ |
| P1.231 | 4-F—Ph | Me | t-Bu | n-Pr | H | 66-68° C. |
| P1.232 | 4-F—Ph | n-Pr | CMe₂Et | Me | H | 56-58° C. |
| P1.233 | 4-F—Ph | Me | t-Bu | n-Bu | H | Gum |
| P1.234 | 4-F—Ph | n-Bu | t-Bu | n-Bu | H | LQ |
| P1.235 | Ph | Me | t-Bu | n-Bu | H | LQ |
| P1.236 | Ph | Me | t-Bu | n-Pr | H | LQ |
| P1.237 | 4-F—Ph | n-Bu | t-Bu | n-Pr | H | LQ |
| P1.238 | Ph | n-Bu | t-Bu | n-Pr | H | LQ |
| P1.239 | 3-Cl—Ph | Et | t-Bu | Et | H | 58-60° C. |
| P1.240 | 3-Cl—Ph | n-Bu | t-Bu | Et | H | LQ |
| P1.241 | 4-F—Ph | n-Pr | t-Bu | n-Pr | H | LQ |
| P1.242 | 4-F—Ph | n-Bu | CMe₂Et | Me | H | LQ |
| P1.243 | Ph | n-Pr | t-Bu | n-Bu | H | 46-48° C. |
| P1.244 | 4-F—Ph | Et | t-Bu | n-Bu | H | LQ |
| P1.245 | 4-Cl—Ph | Me | t-Bu | Et | H | Gum |
| P1.246 | 4-Cl—Ph | n-Bu | t-Bu | Et | H | LQ |
| P1.247 | 3-Cl—Ph | Me | t-Bu | Et | H | LQ |
| P1.248 | 4-F—Ph | n-Pr | t-Bu | n-Bu | H | Gum |
| P1.249 | 2-F—Ph | n-Pr | t-Bu | Et | H | 66-68° C. |
| P1.250 | 3-F—Ph | n-Bu | t-Bu | Et | H | 46-48° C. |
| P1.251 | 2-F—Ph | Me | t-Bu | Et | H | LQ |
| P1.252 | 3-F—Ph | Me | t-Bu | Et | H | LQ |
| P1.253 | 3-F—Ph | n-Pr | t-Bu | Et | H | 64-66° C. |
| P1.254 | 3-Cl—Ph | n-Pr | t-Bu | Et | H | LQ |
| P1.255 | 4-Cl—Ph | n-Pr | t-Bu | Et | H | 48-50° C. |
| P1.256 | 2-F—Ph | n-Bu | t-Bu | Et | H | 72-74° C. |
| P1.257 | 4-Me—Ph | Me | t-Bu | Et | H | Gum |
| P1.258 | 4-Me—Ph | Et | t-Bu | Et | H | Gum |
| P1.259 | Ph | Et | 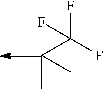 | Me | H | Gum |
| P1.260 | 4-Me—Ph | n-Pr | t-Bu | Et | H | Gum |
| P1.261 | 4-Me—Ph | n-Bu | t-Bu | Et | H | Gum |
| P1.262 | 3-OMe—Ph | n-Pr | t-Bu | Et | H | Gum |
| P1.263 | 3-Me—Ph | n-Pr | t-Bu | Et | H | Gum |
| P1.264 | 3-Me—Ph | n-Bu | t-Bu | Et | H | Gum 4 |
| P1.265 | 4-OMe—Ph | n-Pr | t-Bu | Et | H | 60-62 8 |
| P1.266 | 3-Me—Ph | Et | t-Bu | Et | H | 62-64 |
| P1.267 | 3-OMe—Ph | n-Bu | t-Bu | Et | H | Gum 12 |
| P1.268 | 3-OMe—Ph | Me | t-Bu | Et | H | Gum 16 |
| P1.269 | 3-OMe—Ph | Et | t-Bu | Et | H | Gum |
| P1.270 | 4-OMe—Ph | n-Bu | t-Bu | Et | H | Gum 20 |

TABLE P2
Compounds of formula (I-2)

| | |
|---|---|
| 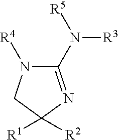 | (I-2) |

The compounds in Table P2 can be prepared as described in the examples above or similar methodology.

| Compound | | | | | | Melting |
|---|---|---|---|---|---|---|
| No. | R1 | R2 | R3 | R4 | R5 | Point |
| P2.1 | 4-F—Ph | 4-F—Ph | t-Bu | Me | H | 99-102° C. |
| P2.2 | 4-F—Ph | c-Pr | t-Bu | Me | H | LQ |

TABLE P3
Compounds of formula (I-3)

| | |
|---|---|
| 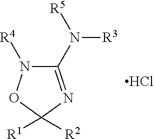 | (I-3) |

The compounds in Table P3 can be prepared in the presence of an acid.

| Compound | | | | | | Melting |
|---|---|---|---|---|---|---|
| No. | R1 | R2 | R3 | R4 | R5 | Point |
| P3.1 | 4-F—Ph | n-propyl | t-Bu | Me | H | 142-144° C. |

### BIOLOGICAL EXAMPLES

### Example B1: Diabrotica balteata (Corn Root Worm)

Maize sprouts placed onto an agar layer in 24-well microtiter plates were treated with aqueous test solutions prepared from 10,000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm:

P1.1, P1.5, P1.6, P1.7, P1.8, P1.9, P1.10, P1.11, P1.12, P1.13, P1.15, P1.17, P1.18, P1.19, P1.20, P1.21, P1.22, P1.23, P1.28, P1.29, P1.30, P1.32, P1.33, P1.34, P1.35, P1.36, P1.37, P1.38, P1.42, P1.43, P1.44, P1.46, P1.47, P1.48, P1.49, P1.50, P1.51, P1.52, P1.53, P1.54, P1.55, P1.56, P1.57, P1.58, P1.59, P1.60, P1.61, P1.62, P1.63, P1.64, P1.65, P1.66, P1.67, P1.70, P1.71, P1.72, P1.74, P1.75, P1.77, P1.80, P1.81, P1.82, P1.84, P1.85, P1.86, P1.87, P1.88, P1.89, P1.9 P1.4, P1.95, P1.98, P1.99, P1.100, P1.102, P1.104, P1.105, P1.106, P1.107, P1.108, P1.110, P1.111, P1.112, P1.113, P1.114, P1.116, P1.117, P1.120, P1.121, P1.122, P1.123, P1.124, P1.125, P1.126, P1.127, P1.128, P1.129, P1.130, P1.131, P1.132, P1.133, P1.134, P1.135, P1.136, P1.137, P1.139, P1.140, P1. 141, P1.142, P1.143, P1.145, P1.146, P1.147, P1.148, P1.149, P1.150, P1.153, P1.154, P1.155, P1.158, P1.159, P1.170, P1.171, P1.172, P1.173, P1.174, P1.175, P1.176, P1.177, P1.178, P1.179, P1.180, P1.182, P1.183, P1.184, P1.185, P1.186, P1.187, P1.188, P1.190, P1.191, P1.192, P1.194, P1.195, P1.196, P1.197, P1.200, P1.201, P1.202, P1.203, P1.204, P1.205, P1.206, P1.207, P1.208, P1.210, P1.211, P1.212, P1.213, P1.215, P1.216, P1.219, P1.220, P1.223, P1.224, P1.232, P1.241, P1.242, P1.244, P1-259.

### Example B2: Euschistus heros (Neotropical Brown Stink Bug)

Soybean leaves on agar in 24-well microtiter plates were sprayed with aqueous test solutions prepared from 10,000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm:

P1.22, P1.23, P1.29, P1.77, P1.87, P1.88, P1.89, P1.119, P1.188, P1.200, P1.216, P1.232, P1.242, P1.248.

### Example B3: Myzus persicae (Green Peach Aphid): Feeding/Contact Activity

Sunflower leaf discs were placed onto agar in a 24-well microtiter plate and sprayed with aqueous test solutions prepared from 10,000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P1.138.

### Example B4: Myzus persicae (Green Peach Aphid): Intrinsic Activity

Test compounds prepared from 10,000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.

The following compounds resulted in at least 80% mortality at a test rate of 12 ppm:

P1.9, P1.17, P1.42, P1.44, P1.55, P1.60, P1.65, P1.70, P1.88, P1.98, P1.100, P1.152, P1.169, P1.171, P1.190, P1.201, P1.207, P1.259.

### Example B4: Plutella xylostella (Diamond Back Moth)

24-well microtiter plates with artificial diet were treated with aqueous test solutions prepared from 10,000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm:

P1.1, P1.2, P1.4, P1.5, P1.6, P1.7, P1.12, P1.13, P1.17, P1.22, P1.23, P1.24, P1.34, P1.36, P1.37, P1.39, P1.46, P1.47, P1.50, P1.51, P1.52, P1.55, P1.57, P1.59, P1.62, P1.63, P1.70, P1.71, P1.72, P1.73, P1.74, P1.77, P1.80, P1.86, P1.87, P1.88, P1.89, P1.91, P1.98, P1.100, P1.104, P1.106, P1.108, P1.109, P1.110, P1.115, P1.119, P1.120, P1.122, P1.124, P1.125, P1.144, P1.162, P1.163, P1.164, P1.166, P1.167, P1.168, P1.169, P1.170, P1.171, P1.172, P1.178, P1.188, P1.190, P1.192, P1.194, P1.200, P1.201, P1.203, P1.204, P1.207, P1.208, P1.210, P1.216, P1.219, P1.220, P1.223, P1.224, P1.232, P1.242, P1.253.

### Example B5: Spodoptera littoralis (Egyptian Cotton Leaf Worm)

Cotton leaf discs were placed onto agar in 24-well microtiter plates and sprayed with aqueous test solutions prepared from 10,000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P1.1, P1.2, P1.3, P1.4, P1.5, P1.6, P1.9, P1.13, P1.14, P1.17, P1.22, P1.23, P1.24, P1.25, P1.28, P1.29, P1.32, P1.34, P1.35, P1.36, P1.42, P1.44, P1.47, P1.50, P1.51, P1.52, P1.54, P1.55, P1.56, P1.59, P1.60, P1.61, P1.63, P1.64, P1.65, P1.66, P1.70, P1.72, P1.74, P1.75, P1.77, P1.88, P1.89, P1.93, P1.98, P1.100, P1.104, P1.106, P1.108, P1.115, P1.117, P1.118, P1.120, P1.122, P1.124, P1.125, P1.128, P1.129, P1.144, P1.158, P1.163, P1.164, P1.166, P1.167, P1.169, P1.170, P1.171, P1.173, P1.179, P1.180, P1.182, P1.184, P1.186, P1.188, P1.189, P1.190, P1.191, P1.192, P1.197, P1.201, P1.206, P1.207, P1.208, P1.210, P1.216, P1.219, P1.220, P1.223, P1.224, P1.229, P1.230, P1.232, P1.242, P1.255.

### Example B6: Spodoptera littoralis (Egyptian Cotton Leaf Worm)

Test compounds were applied by pipette from 10,000 ppm DMSO stock solutions into 24-well plates and mixed with agar. Lettuce seeds were placed onto the agar and the multi well plate was closed by another plate which contained also agar. After 7 days the compound was absorbed by the roots and the lettuce grew into the lid plate. The lettuce leaves were then cut off into the lid plate. Spodoptera eggs were pipetted through a plastic stencil onto a humid gel blotting paper and the lid plate was closed with it. The samples were assessed for mortality, anti-feedant effect and growth inhibition in comparison to untreated samples 6 days after infestation.

The following compounds gave an effect of at least 80% in at least one of the three categories (mortality, anti-feeding, or growth inhibition) at a test rate of 12.5 ppm:

P1.55, P1.88, P1.89, P1.100, P1.119, P1.126, P1.164, P1.179, P1.180, P1.201, P1.207, P1.220, P1.222, P1.224, P1.232, P1.242.

### Example B7: Tetranychus urticae (Two-Spotted Spider Mite): Feeding/Contact Activity

Bean leaf discs on agar in 24-well microtiter plates were sprayed with aqueous test solutions prepared from 10,000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm:

P1.1, P1.3, P1.4, P1.5, P1.9, P1.13, P1.35, P1.50, P1.52, P1.54, P1.59, P1.60, P1.61, P1.65, P1.72, P1.84, P1.85, P1.86, P1.88, P1.94, P1.142, P1.145, P1.151, P1.158, P1.167, P1.170, P1.171, P1.184, P1.201, P1.203, P1.208, P1.219, P1.224, P1.238, P1.250, P1.253, P1.257.

### Example B8: Thrips tabaci (Onion Thrips): Feeding/Contact Activity

Sunflower leaf discs were placed on agar in 24-well microtiter plates and sprayed with aqueous test solutions prepared from 10,000 ppm DMSO stock solutions. After drying the leaf discs were infested with a thrips population of mixed ages. The samples were assessed for mortality 6 days after infestation.

The following compounds resulted in at least 80% mortality at an application rate of 200 ppm:

P1.2, P1.4, P1.5, P1.7, P1.9, P1.36, P1.42, P1.56, P1.59, P1.61, P1.74, P1.90, P1.91, P1.219.

### Example B9: Aedes aegypti (Yellow Fever Mosquito)

Test solutions, at an application rate of 200 ppm in ethanol, were applied to 12 well tissue culture plates. Once the deposits were dry, five, two to five day old adult female Aedes aegypti were added to each well, and sustained with a 10% sucrose solution in a cotton wool plug. Assessment of knockdown was made one hour after introduction, and mortality was assessed at 24 and 48 hours after introduction.

The following compounds gave at least 80% control of Aedes aegypti after 48 h and/or 24 h:

P1.1, P1.2, P1.3, P1.4, P1.5, P1.6, P1.7, P1.8, P1.9, P1.10, P1.11, P1.12, P1.13, P1.14, P1.15, P1.16, P1.17, P1.18, P1.19, P1.20, P1.21, P1.22, P1.23, P1.24, P1.25, P1.26, P1.27, P1.28, P1.29, P1.30, P1.31, P1.32, P1.33, P1.34, P1.35, P1.36, P1.37, P1.38, P1.39, P1.40, P1.41, P1.42, P1.43, P1.44, P1.45, P1.46, P1.47, P1.48, P1.49, P1.50, P1.51, P1.52, P1.53, P1.54, P1.55, P1.56, P1.57, P1.58, P1.59, P1.60, 6 P1.1, P1.62, P1.63, P1.64, P1.65, P1.66, P1.67, P1.69, P1.70, P1.71, P1.72, P1.73, P1.74, P1.75, P1.76, P1.77, P1.78, P1.79, P1.80, P1.81, P1.82, P1.83, P1.84, P1.85, P1.86, P1.87, P1.88, P1.89, P1.90, P1.91, P1.92, P1.93, P1.94, P1.95, P1.96, P1.97, P1.98, P1.99, P1.100, P1.101, P1.102, P1.103, P1.104, P1.105, P1.106, P1.107, P1.108, P1.154, P1.155, P1.156, P1.157, P1.158, P1.159, P1.160, P1.161, P1.162, P1.163, P1.164, P1.165, P1.166, P1.167, P1.168, P1.169, P1.170, P1.171, P1.172, P1.173, P1.174, P1.175, P1.176, P1.177, P1.178, P1.179, P1.180, P1.181, P1.182, P1.183, P1.184, P1.185, P1.186, P1.187, P1.188, P1.189, P1.190, P1.191, P1.192, P1.193, P1.194, P1.195, P1.196, P1.197, P1.198, P1.199, P1.200, P1.201, P1.202, P1.204, P1.205, P1.206, P1.207, P1.208, P1.210, P1.211, P1.213, P1.214, P1.215, P1.216, P1.217, P1.218, P1.221, P1.223, P1.228, P1.229, P1.230, P1.232, P1.235, P1.237, P1.238, P1.240, P1.242, P1.245, P1.248, P1.250, P1.254, P1.255, P1.256, P1.259, P2.1, P2.2.

### Example B10: Anopheles stephensi (Indian Malaria Mosquito)

Test solutions, at an application rate of 200 ppm in ethanol, were applied to 12 well tissue culture plates. Once the deposits were dry, five, two to five day old adult female Anopheles stephensi were added to each well, and sustained with a 10% sucrose solution in a cotton wool plug. Assessment of knockdown was made one hour after introduction, and mortality was assessed at 24 and 48 hours after introduction.

The following compounds gave at least 80% control of Anopheles stephensi after 48 h and/or 24 h:

P1.2, P1.3, P1.4, P1.5, P1.6, P1.7, P1.8, P1.9, P1.10, P1.11, P1.12, P1.13, P1.15, P1.16, P1.17, P1.18, P1.19, P1.20, P1.21, P1.22, P1.28, P1.29, P1.34, P1.35, P1.36, P1.37, P1.38, P1.39, P1.40, P1.41, P1.42, P1.43, P1.44, P1.46, P1.47, P1.48, P1.50, P1.51, P1.52, P1.53, P1.54, P1.55, P1.56, P1.58, P1.59, P1.60, P1.61, P1.62, P1.63, P1.64, P1.65, P1.66, P1.67, P1.69, P1.70, P1.71, P1.72, P1.73, P1.74, P1.75, P1.77, P1.79, P1.80, P1.81, P1.82, P1.84, P1.85, P1.86, P1.87, P1.88, P1.89, P1.90, P1.91, P1.92, P1.93, P1.94, P1.96, P1.98, P1.99, P1.100, P1.101, P1.104, P1.105, P1.106, P1.107, P1.108, P1.154, P1.155, P1.156, P1.158, P1.159, P1.160, P1.161, P.1.162, P1.163, P1.164, P1.165, P1.166, P1.167, P1.168, P1.169, P1.170, P1.171, P1.172, P1.173, P1.174, P1.175, P1.176, P1.178, P1.179, P1.180, P1.181, P1.182, P1.183, P1.184, P1.185, P1.186, P1.187, P1.188, P1.189, P1.190, P1.191, P1.192, P1.193, P1.194, P1.195, P1.196, P1.197, P1.198, P1.199, P1.200, P1.201, P1.202, P1.203, P1.204, P1.205, P1.206, P1.207, P1.208, P1.210, P1.213, P1.214, P1.215, P1.216, P1.217, P1.218, P1.220, P1.221, P1.222, P1.223, P1.224, P1.225, P1.226, P1.227, P1.228, P1.229, P1.230, P1.231, P1.232, P1.233, P1.234, P1.235, P1.236, P1.237, P1.238, P1.239, P1.242, P1.243, P1.244, P1.245, P1.246, P1.247, P1.248, P1.250, P1.254, P1.256, P2.1, P2.2.

### Example B11: Evaluation of Insecticide Impregnated Polymer Surfaces

Preparation of polymeric sheets: Compound P1.17 was impregnated into LDPE (low density polyethylene) by mixing the respective polymer with the compound at high temperatures and the resultant polymeric material was then mould into thin discs or plaques.

Twenty four hours prior to the relevant assessment intervals, three to five day old non-blood fed adult mosquitoes were taken from the culture and lightly anaesthetised with carbon dioxide. Ten females were selected and placed in a 250 ml plastic cup, retained with a net lid, provided with a 10% sucrose solution soaked in a cotton wool bung and held under culturing environmental conditions.

After twenty four hours, impregnated polymer sheets (measuring about 150 mm in diameter and about 0.1 mm in thickness) were removed from storage and wrapped around the glazed side of an 11 cm ceramic tile and held in place with an elastic band. The age of the polymer sheets used were either 1-2 weeks or 10 weeks old. A cup of the pre-selected mosquitoes were again lightly anaesthetised with carbon dioxide and transferred from the holding cup to the base of a 9 cm plastic Petri dish. The relevant impregnated polymer sheet was placed over the Petri dish and held in place with an elastic band. Once the mosquitoes had recovered from the anaesthetic, ca. 1 minute, the impregnated polymer sheet was placed on a holding rack, such that treated side was at an angle of 60° to the horizontal.

After one hour an assessment of mosquito knockdown was made. A mosquito was said to be knocked down if it was unable to right itself once it had fallen over. The mosquitoes were lightly anaesthetised again, and removed from the Petri dish exposure chamber and returned to the holding cups. The mosquitoes were supplied with a 10% sucrose solution soaked in a cotton wool bung and held under culturing environmental conditions. An assessment of mortality was made 24 hours and 48 hours after exposure. A mosquito was said to be dead if it is unable to right itself once it had fallen over.

Compound P1.17 showed at least 80% control of Aedes Aegypti and Anopheles stephensi after 48 h and/or 24 h at a concentration of 2% of active ingredient in LDPE sheets—after 1 week, after 2 weeks and after 10 weeks.

1. The use of one or more compounds of formula (I), for controlling mosquitoes 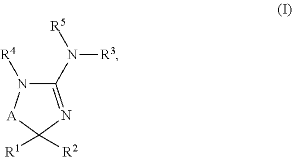
wherein
A represents O or CH₂;
R1 is aryl; optionally substituted aryl; heteroaryl; optionally substituted heteroaryl; or cyclo-propyl;
R2 is C1-C6-alkyl; optionally substituted C1-C6-alkyl; C2-C6-alkenyl; optionally substituted C2-C6-alkenyl; C3-C8-cycloalkyl; C1-C4-alkyl, C1-C4-haloalkyl, halogen or CN substituted C3-C8-cycloalkyl; C3-C8-cycloalkyl in which the cycloalkyl ring contains 1 oxygen heteroatom; optionally substituted C3-C8-cycloalkyl in which the cycloalkyl ring contains 1 oxygen heteroatom; aryl; optionally substituted aryl; arylalkyl; arylalkyl wherein the aryl is substituted by one to three substituents independently selected from C1-C4-alkyl, C1-C4-alkoxy, C1-C4-haloalkyl, halogen and CN; C4-C8-cycloalkylalkyl; or C1-C4-alkyl, C1-C4-haloalkyl, halogen or CN substituted C4-C8-cycloalkylalkyl;
R3 is C1-C8-alkyl; C1-C8-haloalkyl; C2-C8-alkenyl; C2-C8-alkynyl; C2-C8-haloalkenyl; C3-C10-cycloalkyl; C3-C10-halocycloalkyl; C3-C8-cycloalkyl in which the cycloalkyl ring contains 1 oxygen heteroatom; optionally substituted C3-C8-cycloalkyl in which the cycloalkyl ring contains 1 oxygen heteroatom; aryl; or haloaryl;
R4 is H; C1-C6-alkyl; halogen or CN substituted C1-C6-alkyl; C2-C6-alkenyl; or C1-C6-alkoxy;
R5 is H; C1-C6-alkyl; halogen or CN substituted C1-C6-alkyl; C2-C6-alkenyl; C1-C4-alkyl, C1-C4-haloalkyl, halogen or CN substituted C2-C6-alkenyl; C2-C6-alkynyl; or C1-C4-alkyl, C1-C4-haloalkyl, halogen or CN substituted C2-C6-alkynyl; or an agrochemically acceptable salt or N-oxide thereof.

2. The use according to claim 1 wherein A is O, R¹ is aryl; aryl substituted by 1 to 3 substituents independently selected from C₁-C₄-alkyl, C₁-C₄-alkoxy, C₁-C₄-haloalkoxy, C₁-C₄-haloalkyl, halogen and CN; heteroaryl containing 1 to 3 heteroatoms independently selected from nitrogen, oxygen and sulfur; heteroaryl thereof substituted by 1 to 3 substituents independently selected from C₁-C₄-alkyl, C₁-C₄-alkoxy, C₁-C₄-haloalkyl, halogen and CN; or cyclo-propyl; R² is C₁-C₆-alkyl; halogen or CN substituted C₁-C₆-alkyl; C₂-C₆-alkenyl; C₁-C₄-haloalkyl, halogen or CN substituted C₂-C₆-alkenyl; C₃-C₅-cycloalkyl; C₁-C₄-alkyl, C₁-C₄-haloalkyl, halogen or CN substituted C₃-C₅-cycloalkyl; C₃-C₅-cycloalkyl in which the cycloalkyl ring contains 1 oxygen heteroatom; C₁-C₄-alkyl, C₁-C₄-haloalkyl, halogen or CN substituted C₃-C₅-cycloalkyl in which the cycloalkyl ring contains 1 oxygen heteroatom; aryl; aryl substituted by 1 to 3 substituents independently selected from C₁-C₄-alkyl, C₁-C₄-alkoxy, C₁-C₄-haloalkyl, halogen and CN; arylalkyl; arylalkyl wherein the aryl is substituted by one to three substituents independently selected from C₁-C₄-alkyl, C₁-C₄-alkoxy, C₁-C₄-haloalkyl, halogen and CN; C₄-C₈-cycloalkylalkyl; or C₁-C₄-alkyl, C₁-C₄-haloalkyl, halogen or CN substituted C₄-C₈-cycloalkylalkyl; R³ is a branched C₁-C₈-alkyl; C₁-C₈-haloalkyl; C₂-C₈-alkenyl; C₂-C₅-alkynyl; C₃-C₁₀-cycloalkyl; C₃-C₁₀-halocycloalkyl; C₁-C₄-alkyl, C₁-C₄-haloalkyl, halogen or CN substituted C₃-C₅-cycloalkyl in which the cycloalkyl ring contains 1 oxygen heteroatom; aryl; or haloaryl; R⁴ is C₁-C₆-alkyl; halogen or CN substituted C₁-C₆-alkyl; or C₂-C₆-alkenyl; and R⁵ is H; C₁-C₆-alkyl; halogen or CN substituted C₁-C₆-alkyl; C₂-C₆-alkenyl; C₁-C₄-alkyl, C₁-C₄-haloalkyl, halogen or CN substituted C₂-C₆-alkenyl; C₂-C₆-alkynyl; C₁-C₄-alkyl, C₁-C₄-haloalkyl, halogen or CN substituted C₂-C₆-alkynyl; aryl; or C₁-C₄-alkyl, C₁-C₄-haloalkyl, halogen or CN substituted aryl.

3. An integrated mosquito vector control management method or a vector control solution comprising one or more compounds as defined in claim 1.

4. The vector control solution according to claim 3, wherein the solution is a net incorporated with the one or more compounds.

5. The vector control solution according to claim 3, wherein the solution is a composition for coating a net, which composition comprises the one or more compounds.

6. The vector control solution according to claim 3, wherein the solution is a composition for spraying surfaces of a dwelling, which composition comprises the one or more compounds.

7. A compound of formula (I-A1), 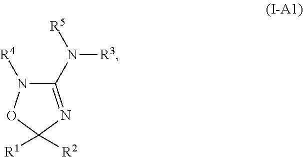
wherein
R¹ is aryl; optionally substituted aryl; heteroaryl; optionally substituted heteroaryl; or cyclo-propyl;
R² is C1-C6-alkyl; optionally substituted C1-C6-alkyl; C2-C6-alkenyl; optionally substituted C2-C6-alkenyl; C3-C8-cycloalkyl; C1-C4-alkyl, C1-C4-haloalkyl, halogen or CN substituted C3-C8-cycloalkyl; C3-C8-cycloalkyl in which the cycloalkyl ring contains 1 oxygen heteroatom; optionally substituted C3-C8-cycloalkyl in which the cycloalkyl ring contains 1 oxygen heteroatom; aryl; optionally substituted aryl; arylalkyl arylalkyl wherein the aryl is substituted by one to three substituents independently selected from C1-C4-alkyl, C1-C4-alkoxy, C1-C4-haloalkyl, halogen and CN; C4-C8-cycloalkylalkyl; or C1-C4-alkyl, C1-C4-haloalkyl, halogen or CN substituted C4-C8-cycloalkylalkyl;
R⁴ is H; C1-C6-alkyl; halogen or CN substituted C1-C6-alkyl; C2-C6-alkenyl; or C1-C6-alkoxy;
R⁵ is H; C1-C6-alkyl; halogen or CN substituted C1-C6-alkyl; C2-C6-alkenyl; C1-C4-alkyl, C1-C4-haloalkyl, halogen or CN substituted C2-C6-alkenyl; C2-C6-alkynyl; or C1-C4-alkyl, C1-C4-haloalkyl, halogen or CN substituted C2-C6-alkynyl; and
R³ is selected from n-C₁-C₈-alkyl, C₃-C₅-fluoroalkyl, C₂-C₅-alkenyl, C₂-C₅-alkynyl, C₂-C₈-haloalkenyl, C₃-C₁₀-cycloalkyl, C₃-C₁₀-halocycloalkyl, C₃-C₁₀-cycloalkyl in which the cycloalkyl ring contains 1 oxygen heteroatom, phenyl, and halophenyl; or
R³ is C1-C8-alkyl; C1-C8-haloalkyl; C2-C8-alkenyl; C2-C8-alkynyl; C2-C8-haloalkenyl; C3-C10-cycloalkyl; C3-C10-halocycloalkyl; C3-C8-cycloalkyl in which the cycloalkyl ring contains 1 oxygen heteroatom; optionally substituted C3-C8-cycloalkyl in which the cycloalkyl ring contains 1 oxygen heteroatom; aryl; or haloaryl.

8. The compound of claim 7, wherein R³ is either C(C₂H₅)₃, or C(CH₃)₂—CH₂—C(CH₃)₃.

9. The compound of claim 7, wherein R¹ is substituted aryl, heteroaryl; optionally substituted heteroaryl; or cycclopropyl; and R³ is selected from CH(CH₃)₂, C(CH₃)₂(C₂H₅), C(CH₃)(C₂H₅)₂, 1-methyl-cyclopropyl and CH₂CF₃.

10. The compound of claim 7, wherein R² is selected from 3-methyl-oxetan-3-yl, CH₂CF₃ and n-pentyl.

11. The compound of claim 7, wherein R¹ is substituted aryl; heteroaryl; optionally substituted heteroaryl; or cyclo-propyl and R² is methyl, ethyl, n-propyl, n-hexyl, phenylethyl, and CF₃.

12. The compound of claim 7, wherein R² is selected from 3-methyl-oxetan-3-yl, CH₂CF₃, n-pentyl, methyl, ethyl, n-propyl, n-hexyl, phenylethyl, and CF₃.

13. The compound of claim 7, wherein R¹ is substituted aryl, heteroaryl, optionally substituted heteroaryl and
(i) R² is n-butyl with the proviso that the substituent on the aryl of R¹ is not 4-chloro or not 4-OCF₃; or
(ii) R² is cyclo-pentyl with the proviso that the substituent on the aryl of R¹ is not 4-chloro.

14. The compound of claim 7, wherein R¹ is substituted aryl, heteroaryl, or optionally substituted heteroaryl and R² is C₃-C₈-cycloalkyl with the proviso that the substituent on the aryl of R¹ is 2-fluoro.

15. The compound of claim 7, wherein R² is cyclo-propylmethyl with the proviso that the substituent on the aryl of R¹ is not 4-chloro.

16. A compound of formula (I-2), 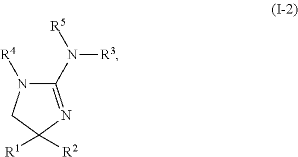
wherein
R¹ is aryl; optionally substituted aryl; heteroaryl; optionally substituted heteroaryl; or cyclo-propyl;
R³ is C1-C8-alkyl; C1-C8-haloalkyl; C2-C8-alkenyl; C2-C8-alkynyl; C2-C8-haloalkenyl; C3-C10-cycloalkyl; C3-C10-halocycloalkyl; C3-C8-cycloalkyl in which the cycloalkyl ring contains 1 oxygen heteroatom; optionally substituted C3-C8-cycloalkyl in which the cycloalkyl ring contains 1 oxygen heteroatom; aryl; or haloaryl;
R⁴ is H; C1-C6-alkyl; halogen or CN substituted C1-C6-alkyl; C2-C6-alkenyl; or C1-C6-alkoxy;
R⁵ is H; C1-C6-alkyl; halogen or CN substituted C1-C6-alkyl; C2-C6-alkenyl; C1-C4-alkyl, C1-C4-haloalkyl, halogen or CN substituted C2-C6-alkenyl; C2-C6-alkynyl; or C1-C4-alkyl, C1-C4-haloalkyl, halogen or CN substituted C2-C6-alkynyl; and
R2 is C1-C6-alkyl; halogen or CN substituted C1-C6-alkyl; C2-C6-alkenyl; optionally substituted C2-C6-alkenyl; C3-C8-cycloalkyl; C1-C4-alkyl, C1-C4-haloalkyl, halogen or CN substituted C3-C8-cycloalkyl; C3-C8-cycloalkyl in which the cycloalkyl ring contains 1 oxygen heteroatom; optionally substituted C3-C8-cycloalkyl in which the cycloalkyl ring contains 1 oxygen heteroatom; aryl; optionally substituted aryl; arylalkyl; arylalkyl wherein the aryl is substituted by one to three substituents independently selected from C1-C4-alkyl, C1-C4-alkoxy, C1-C4-haloalkyl, halogen and CN; C4-C8-cycloalkylalkyl; or C1-C4-alkyl, C1-C4-haloalkyl, halogen or CN substituted C4-C8-cycloalkylalkyl.

17. A pesticidal composition comprising a compound defined in claim 7, one or more formulation additives and a carrier.

18. A combination of active ingredients comprising a compound defined in claim 7, and one or more further active ingredients.

19. A method of controlling insects, acarines, nematodes or molluscs which comprises applying an insecticidally, acaricidally, nematicidally or molluscicidally effective amount of the compound or a composition containing the compound defined in claim 7, to a pest, a locus of pest, a plant, to a plant susceptible to attack by a pest or to plant propagation material thereof, such as a seed, provided if the control were on a human or animal body, then it is non-therapeutical.

20. A plant propagation material comprising by way of treatment or coating one or more compounds defined in claim 1, optionally also comprising a colour pigment.
